(19) United States
(12) Patent Application Publication
(10) Pub. No.: US 2025/0099571 A1
(43) Pub. Date: Mar. 27, 2025

(54) HIV-1 ENVELOPE STABILIZING MUTATIONS

(71) Applicant: Duke University, Durham, NC (US)

(72) Inventors: Rory HENDERSON, Durham, NC (US); S. Munir ALAM, Durham, NC (US); Barton F. Haynes, Durham, NC (US); Kevin J. Wiehe, Durham, NC (US)

(21) Appl. No.: 18/906,155

(22) Filed: Oct. 3, 2024

Related U.S. Application Data
(62) Division of application No. 17/281,918, filed on Mar. 31, 2021, now Pat. No. 12,138,304.
(60) Provisional application No. 62/739,727, filed on Oct. 1, 2018.

(51) Int. Cl. A61K 39/12; C07K 14/16; C12N 7/00; C12N 15/63

(52) U.S. Cl. CPC A61K 39/12; C07K 14/162; C12N 7/00; C12N 15/63; C12N 2740/16122; C12N 2740/16134

(57) ABSTRACT

The technology is directed to HIV envelopes which comprise sequence modifications wherein these modifications prevent CD4-induced transitions of the HIV Env. Provided also are compositions comprising envelopes of the technology, and methods of use.

This application is a divisional application of U.S. Ser. No. 17/281,918, filed Mar. 31, 2021, which is a 35 U.S.C. 371 national application of international application No. PCT/US2019/049662, filed Sep. 5, 2019, which claims the benefit and priority of U.S. Application Ser. No. 62/739,727 filed Oct. 1, 2018. The contents of these applications are incorporated herein by reference in their entirety.

This invention was made with government support under Center for HIV/AIDS Vaccine Immunology-Immunogen Design grant UM1-AI100645 from the NIH, NIAID, Division of AIDS. The government has certain rights in the invention.

### SEQUENCE LISTING

The instant application contains a Sequence Listing which has been submitted electronically in ASCII format and is hereby incorporated by reference in its entirety. Said ASCII copy, created Sep. 16, 2024, is named and is 128 KB in size.

### TECHNICAL FIELD

The present technology relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The technology also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain aspects the technology provides envelopes designs comprising mutations which stabilize the envelope trimer. In certain embodiments, the stabilized trimer displays antigenic profile similar to the antigenic profile of a BMS-626529 bound trimer. In certain embodiments, the envelopes could comprise additional trimer stabilizing mutations, including but not limited to various SOSIP mutations.

In certain aspects the technology provides SOSIP and membrane bound mutant Envs that display antigenic profiles matching those of the BMS-626529 bound trimer. The BMS compound is known to stabilize state 1 of the virus bound Env and to prevent CD4 induced exposure of non-neutralizing epitopes. The relevant regions in or near the BMS compound's binding site include the β20-β21 loop, a site of CD4 contact playing an important role in CD4 triggering, and the layer-1 and layer-2 elements of gp120, which are involved in gp120 association with gp41. Layer-1 and layer-2 are known to play a role in mediating Env transitions.

In certain aspects the envelopes are designed by a three-pronged approach toward replicating the BMS compound's effect on the envelope trimer via mutation (specific amino acid changes). Specifically, we introduced space filling mutations in the BMS binding site, layer-1 and layer-2 blocking mutations, in addition to V3 locking mutations via increasing V1/V2 to V3 hydrophobic contacts. We began our screen and characterization in the BG505 SOSIP as this allows for a detailed evaluation of the mutation effects on specific sites. Our screening ultimately identified stabilizing residues in layers 1 and 2, in V1/V2, and in V3.

In certain embodiments, the locations of the mutations are depicted here on gp120 and labeled as F14, for layer-1 and layer-2 locking mutations, in the lime green and purple regions, and Vt8, for V1/V2 to V3 coupling mutations, in the green and red regions. See e.g. FIG. 1, FIG. 15. In non-limiting embodiments, inventive envelopes comprise F14 mutations (See e.g. FIG. 15), Vt8 mutations (See e.g. FIG. 15), or the combination F14/Ft8. In non-limiting embodiments, envelopes carrying these mutations are stable, as these mutations prevented CD4-induced transitions of the HIV-1 Env.

In certain embodiments, the stabilized envelopes have improved antigenic, and/or immunogenic properties.

In certain aspects the technology provides a recombinant HIV-1 envelope comprising a set of mutations selected from sets F1-F14, Vt1-Vt8 mutations in Ex. 1, FIG. 15, or any combination or subcombination within a set. In non-limiting embodiments, the envelopes could be designed to multimerize. Non-limiting embodiments of multimers include nano-particles based on ferritin. In certain embodiments, ferritin could be fused to the envelope or attached via a sortase reaction.

In certain embodiments, the envelope is a protomer, and the three protomers form a trimer stabilized by the presence of the mutations. In certain embodiments, the trimer could be soluble (e.g. but not limited to a SOSIP trimer) or membrane bound.

In certain embodiments, any envelope, including but not limited to BG505, CONs, JRFL, CH505 T/F, w.53. M5, M5G458mut, M11, CH848 10.17 DT, 19CV3 could be designed to carry mutations as described.

In certain embodiments, the envelopes could comprise additional stabilizing mutations, including but not limited to various SOSIP mutations.

In certain embodiments, the stabilized trimer displays an antigenic profile similar to the antigenic profile of a BMS-626529 bound trimer.

In certain embodiments, the envelope can be any of the recombinant proteins listed in Table 1, FIGS. 41-54. In certain embodiments, a nucleic acid encodes any of the recombinant envelopes of the technology, e.g. as listed in Table 1, FIGS. 41-54. In certain embodiments the envelopes are membrane bound gp160 envelope.

In certain embodiments, the envelope is provided as an immunogenic composition comprising any one of the envelopes of the technology and a carrier. In certain embodiments, the immunogenic composition further comprises an adjuvant.

In certain embodiments, the technology is a method of inducing an immune response in a subject comprising administering an immunogenic composition comprising any one of the stabilized envelopes of the technology. In certain embodiments, the composition is administered as a prime and/or a boost. In certain embodiments, the composition comprises nanoparticles.

In certain embodiments, the technology is a composition comprising a plurality of nanoparticles comprising a plurality of the envelopes/trimers of the technology. In non-limiting embodiments, the envelopes/trimers of the technology are multimeric when comprised in a nano-particle. In certain embodiments, the nanoparticle size is suitable for a delivery in a pharmaceutical composition. In non-liming embodiments the nanoparticles are ferritin based nano-particles.

In certain embodiments, the technology provides compositions and methods for induction of immune response, for example cross-reactive (broadly) neutralizing (bn) Ab induction. In certain embodiments, the methods use compositions comprising newly designed HIV-1 immunogens which bind to precursors, and/or UCAs of different HIV-1 bnAbs. In certain embodiments, these are UCAs of V1V2 glycan and V3 glycan antibodies.

In certain aspects the technology provides a recombinant HIV-1 envelope comprising a set of mutations selected from sets F1-F14, sets Vt1-Vt8 mutations in FIG. 15, any combination of sets from F1-F14 and Vt1-Vt8, or subcombination of mutations within a set.

In certain embodiments the set of mutations is F14 (FIG. 15).

In certain embodiments the set of mutations is Vt8 (FIG. 15).

In certain embodiments the combination of sets of mutations is F14 and Vt8 (FIG. 15).

In certain aspects the technology provides a recombinant HIV-1 envelope, wherein the envelope is a protomer, and wherein three protomers form a trimer stabilized by the presence of the mutations. In certain embodiments, the trimer could comprise additional stabilizing mutations, including but not limited to various SOSIP mutations.

In certain embodiments the envelope is BG505, CONs, JRFL, CH505 T/F, w.53., CH505 M5, CH505 M5G458mut, CH505 M11, CH848 10.17 DT, or 19CV3. Any envelope could be modified to comprise the amino acid changes described.

In certain embodiments the envelope comprises additional stabilizing mutations.

In certain embodiments the envelope form stabilized trimers which display antigenic profile similar to the antigenic profile of a BMS-626529 bound trimer.

In certain aspects the technology provides a nucleic acid encoding any of the recombinant HIV-1 envelopes of the preceding claims.

In certain embodiments the nucleic acid encodes a recombinant HIV-1 envelope which is membrane bound gp160 envelope.

In certain aspects the technology provides an immunogenic composition comprising the recombinant HIV-1 envelope of any one of the preceding claims and a carrier.

In certain embodiments the immunogenic compositions further comprise an adjuvant.

In certain embodiments the immunogenic compositions comprise a recombinant HIV-1 envelope in a nanoparticle.

In certain aspects the technology provides an immunogenic composition comprising a nucleic acid encoding a recombinant HIV-1 envelope of the technology and a carrier.

In certain aspects the immunogenic compositions comprising a nucleic acid further comprise an adjuvant.

In certain aspects the technology provides methods of inducing an immune response in a subject comprising administering inventive envelopes, nucleic acid or immunogenic compositions. In certain embodiments the methods further comprise administering an adjuvant.

### BRIEF DESCRIPTION OF THE DRAWINGS

FIGS. 1A-1F. Disrupting HIV-1 Env allostery to block CD4-induced conformational changes. FIG. 1A shows the HIV-1 Env SOSIP structure. Protomer to the left colored according to regions of allosteric control with BMS-626529 are represented as black spheres. Protomer to the right in matte color scheme depicts the location of Vt-series mutations (cyan spheres) and F-series mutations (blue spheres). BMS-626529 is depicted in stick representation. FIG. 1B shows the set of Vt8 residues. FIG. 1C shows the set of F14 mutations. Residue sidechains are represented as sticks with Cα atoms represented as cyan spheres. FIG. 1D shows the sequence alignment of BG505 WT (SEQ ID NOS 11-13 and 11-13, respectively, in order of appearance), F14 (SEQ ID NOS 14-16 and 14-16, respectively, in order of appearance), and F14/Vt8 (SEQ ID NOS 17-19 and 17-19, respectively, in order of appearance) with F14 mutations highlighted in blue and Vt8 mutations highlighted in cyan. Layers 1 and 2, the V1/V2 region, and β20-β21 are highlighted using the same color scheme as in FIG. 1A. FIG. 1E shows the binding antigenicity of BG505 SOSIP.664 and mutants. (left) Heatmap of bnAb binding responses, CD4 binding, and CD4 triggering. Values for CD4 binding and CD4 triggering are normalized to WT. (middle) Surface representation of a closed state SOSIP (PDB ID 5CEZ) and (right) an open state structure (PDB ID 5VN3). Colors identify antibody binding locations. FIG. 1F shows the sequences correspond to the aligned sequences in FIG. 1D. F14 and Vt8 mutations are in bolded.

FIGS. 2A-D show physical stability of BG505 SOSIP.664 and mutants. Differential scanning calorimetry data for BG505 parent, F14, Vt8, and F14/Vt8 SOSIPs. Data presented are representative of three independent measures.

FIGS. 3A-3J shows structural details of BG505 F14 SOSIP and BG505 F14/Vt8 SOSIP. FIG. 3A shows refined cryo-EM map of BG505 F14 SOSIP (gp120 in blue and gp41 in orange) bound to VRC01 (purple). FIG. 3B shows refined cryo-EM map of BG505 F14/Vt8 SOSIP (gp120 in blue and gp41 in orange) bound to VRC03 (red) and 10-1074 (green). FIG. 3C shows alignment of BG505 F14 SOSIP gp140 and BG505 F14/Vt8 SOSIP gp140. RMSD is for the alignment of gp120 and gp41 together. FIG. 3D shows cryo-EM map of the BG505 F14/Vt8 construct with fitted coordinates depicting the F14 mutation sites. FIG. 3E shows alignment of the BG505 F14/Vt8 SOSIP gp120 with the BG505 WT SOSIP gp120 showing the relative positions of the F14 mutations. FIG. 3F shows cryo-EM map of the BG505 F14/Vt8 construct with fitted coordinates depicting the Vt8 mutations. FIG. 3G shows alignment of the BG505 F14/Vt8 SOSIP gp120 with the BG505 WT SOSIP gp120 showing the relative positions of the Vt8 mutation sites. FIGS. 3H and 3I show transparent cryo-EM maps with ribbon representations of refined coordinates for BG505 F14 and F14/Vt8 SOSIPs (gp120 in blue and gp41 in orange) identifying gp41 HR1 helical extension. FIG. 3J shows alignment of gp41 three-helix bundle helix (residues 571-596) of BG505 F14 and F14/Vt8 SOSIPs (light orange and orange, respectively), an open state BG505 SOSIP (PDB ID 5VN3; green), and a closed state BG505 SOSIP (PDB ID 5CEZ; blue) identifying the extension of the gp41 HR1.

FIGS. 4A-4F show atomic level structural details of BG505 F14/Vt8 SOSIP. FIG. 4A (top) shows side view of VRC01 bound BG505 F14 SOSIP highlighting the location of interior conformational change. (bottom) Cryo-EM map (mesh) depicting position of gp41 residues K567 and W571. FIG. 4B (top) shows VRC01 bound BG505 F14 SOSIP apex facing trimer orientation highlighting the location of interior conformational change. (bottom) Cryo-EM map (mesh) depicting the apparent histidine triad. FIG. 4C shows side view of histidine triad relative to gp120. FIG. 4D shows top down view of histidine triad relative to gp120. FIGS. 4E & 4F show comparison between the BG505 F14/Vt8 and WT SOSIP layer-1/2 and gp41 HR1 conformations.

FIGS. 5A-5F show domain organization and immunogenicity of the BG505 F14 and BG505 F14/Vt8 SOSIP FIG. 5A shows alignment of closed (PDB ID 5CEZ), partially open (Openₚ; PDB IDs 6CM3 and 6EDU), and open (PDB IDs 5VN3 and 5VN8) gp41 residues 597 to 664. FIG. 5B shows closed (PDB ID 5CEZ), partially open (PDB IDs 6CM3), and open (PDB IDs 5VN3) gp120 relative orientations from alignment of gp41 residues 597 to 664. (inset, left) Relative gp120 orientations from the gp41 597 to 664 residue alignment depicting the location of the K46/K490 hinge-point. FIG. 5C shows cartoon representations of gp120 (light blue), gp120 V1/V2 region (green), and gp41 (yellow) in the closed, partially open, and open states. Letters indicate location of centroids with arrows depicting vectors between centroids. The reduced size of the V1/V2 region in the partially open state represents rearrangement in this region while the green outline of V1/V2 in the open state indicates complete dissociation and disorder. FIG. 5D shows a graph depicting the gp41 W571-W596, W596-K46/K490 centroid, and K46/K490-gp120 centroid vector dihedral (x-axis) vs. W571-W596. W596-K46/K490 centroid, and K46/K490-gp120 centroid vector dihedral using the W596-K46/K490 centroid vector to W571-W596 projection. The BG505 F14 and F14/Vt8 structures are shown as green circles, with the closed state SOSIPs shown as blue dots, the PGT151 bound trimer domains shown as red Xs, the single CD4 bound domains as yellow Xs, and the open and partially open structures shown as orange triangles. The dashed line represents a linear fitting of the closed state SOSIP structure dihedrals. FIG. 5E shows principal components analysis of centroid vectors with principal component one and two eigenvalues plotted for each closed state SOSIP analyzed. Points are colored according to k-means clusters (k=3). FIG. 5F (left) shows alignment of the BG505 F14/Vt8 coordinates with the vFP7.04 bound BG505 DS SOSIP cryo-EM map (EMD-7621). (right) Alignment of the BG505 F14/Vt8 coordinates with the PDB ID 5CEZ map. W571 (sticks) aligns with density corresponding to 5CEZ V570) coordinates.

FIGS. 6A-6E show F14/Vt8 mutations stabilize native, membrane-bound Env. FIG. 6A shows the percentage of cells positive for binding of bnAbs N6, CH01, PGT125, and PGT145 to 293F cell surface expressed BG505, BG505 DS, and BG505 F14/Vt8 trimers. FIG. 6B shows a chart of the percentage of positive cells from each independent triggering experiment colored according to the first (red), second (green) and third (blue) experiments with BG505 represented as squares, BG505 DS represented as upward oriented triangles, and BG505 F14/Vt8 represented as downward oriented triangles. FIG. 6C shows single molecular FRET distribution for BG505 WT (dashed cyan line) and F14/Vt8 (solid blue line) virus Env. FIG. 6D shows single molecular FRET distribution for BG505 WT (dashed pink line) and F14/Vt8 (solid red line) virus Env in the presence of dodecameric CD4. FIG. 6E shows a mechanism for transition between a closed state trimer to an open state trimer.

FIGS. 7A-7B show structure of a closed and open state HIV-1 Env gp140 SOSIP protomer. FIG. 7A (left) shows a closed state SOSIP gp140 protomer (PDB ID 5CEZ) identifying key structural elements. (right) Elements of the closed state allosteric network including 20-21 (yellow), layer-1/2 (lime green/purple), V1/2/3 (green/red) and tryptophans 69, 112 and 427. FIG. 7B (left) shows an open state SOSIP gp140 protomer (PDB ID 5VN3) identifying key structural elements. (right) Elements of the open state allosteric network including 20-21 (yellow), layer-1/2 (lime green/purple). V1/2/3 (green/red) and tryptophans 69, 112 and 427.

FIGS. 8A-8C show BG505 SOSIP Mutant Screening Results. FIG. 8A shows transfection cell supernatant BLI heatmap for F-series, Vt-series, WT SOSIP, WT gp120, BMS-626529 bound BG505 SOSIP, and controls binding to PGT145, 19B, 17B, and VRC01. Results plotted as mean response (nm) from triplicate transfection supernatant screening. FIG. 8B (chart) shows representative size exclusion chromatogram from the BG505 F14 SOSIP purification post PGT145 affinity column purification. (inset) Representative non-reducing SDS-PAGE gel for the BG505 SOSIP.664 mutants. Lanes 1-3 include protein marker, Ferritin, and Thyroglobulin, respectively. Lane 4 contains the SOSIP trimer. FIG. 8C shows negative-stain EM two-dimensional class averages for purified BG505 WT, F14, Vt8, and F14/Vt8 SOSIPs.

FIGS. 9A-9C show BG505 SOSIP Mutant bnAb Binding, CD4 binding, and CD4 Triggering. FIG. 9A (left) show dose response curves for binding of VRC01, PGT121m PG9, PGT145, PGT151, and VRC26 to the BG505 WT, F14, Vt8, and F14/Vt8 SOSIPs. Results plotted as mean response (nm) from duplicate experiments (mean standard error is within plotted points). Lines represent one-site specific fitting of BLI binding data in Prism. The envelopes are BG505 WT, F14, Vt8, and F14/Vt8 SOSIP. FIG. 9B shows representative SPR titrations for BG505 WT, F14, Vt8, and F14/Vt8 SOSIP binding to CD4-Ig. FIG. 9C shows representative CD4 triggering results for BG505 WT (red), F14 (orange), Vt8 (Vt8), and F14/Vt8 (blue) SOSIP. Responses are normalized to BG505 WT SOSIP.

FIGS. 10A-10J show cryo-EM data analysis for BG505 F14 and F14/Vt8 SOSIP datasets. FIGS. 10A and 10F show representative micrographs showing particles selected for refinement from BG505-F14-SOSIP (FIG. 10A), and BG505-F14/Vt8-SOSIP (FIG. 10F) datasets. FIGS. 10B and 10G show distribution of cisTEM score values assigned to particles extracted from datasets BG505-F14-SOSIP (FIG. 10B), and BG505-F14/Vt8-SOSIP (FIG. 10G). FIGS. 10C and 10H show cryo-EM map (solid) with superimposed shape mask (transparent) used during 3D refinement for BG505-F14-SOSIP (FIG. 10C), and BG505-F14/Vt8-SOSIP (FIG. 10H) datasets. FIGS. 10D and 10I show Fourier Shell Correlation curves between half-maps showing estimated resolution according to the 0.143-cutoff criteria (dashed line) for BG505-F14-SOSIP (FIG. 10D) and BG505-F14/Vt8-SOSIP (FIG. 10I) reconstructions. FIGS. 10E and 10J show local map resolution determined using RELION-3.0 for reconstructions of BG505-F14-SOSIP (FIG. 10E) and BG505-F14/Vt8-SOSIP (FIG. 10J).

FIGS. 11A-11D show Cryo-EM fit of BG505 F14 mutations. FIG. 11A shows Cryo-EM map of the BG505 F14 construct with fitted coordinates depicting the F14 mutation sites. FIG. 11B shows alignment of the BG505 F14 SOSIP gp120 with the BG505 WT SOSIP (PDB ID 5CEZ) gp120 showing the relative positions of the F14 mutations. FIG. 11C shows Cryo-EM map (mesh) depicting H66, H72, and H565. FIG. 11D shows Cryo-EM map (mesh) depicting position of gp41 residues K567 and W571.

FIGS. 12A-12C show Cryo-EM fit of BG505 F14/Vt8 SOSIP coordinates to vFP bound BG505 DS SOSIP cryo-EM maps. FIG. 12A show alignment of the BG505 F14/Vt8 coordinates with the vFP20.01 bound BG505 DS SOSIP cryo-EM map (EMD-7459). FIG. 12B shows alignment of the BG505 F14/Vt8 coordinates with the vFP16.02 bound BG505 DS SOSIP cryo-EM map (EMD-7460). FIG. 12C shows alignment of the BG505 F14/Vt8 coordinates with the vFP1.01 bound BG505 DS SOSIP cryo-EM map (EMD-7622).

FIG. 13 shows flow cytometry gating strategy. Example data depicting the gating strategy used to assess Env cell surface expressed gp160 trimer binding to bnAbs and non-bnAbs.

FIGS. 14A-G show antigenicity and triggering of BG505 gp160 construct cell-surface expressed trimer. FIG. 14A shows percentage of cells positive for binding of bnAbs N6, CH01, PGT125, and PGT145 to 293F cell surface expressed gp160 BG505, BG505 F14/Vt8, BG505 F14, and BG505 Vt8 trimers. FIG. 14B shows MFI data for binding of N6 to gp160 BG505, BG505 DS, and BG505 F14/Vt8 trimers. FIG. 14C shows MFI data for binding of N6 to gp160 BG505, BG505 F14/Vt8, BG505 F14, and BG505 Vt8 trimers. FIG. 14D shows percentage of cells positive for binding of non-bnAbs 17B and 19B to 293F cell surface expressed BG505, BG505 DS, and BG505 F14/Vt8 trimers in the presence and absence of sCD4 or CD4-Ig. FIG. 14E shows MFI data for binding of non-bnAbs 17B and 19B to 293F cell surface expressed BG505,BG505 DS, and BG505 F14/Vt8 trimers in the presence and absence of sCD4 and CD4-Ig. FIG. 14F shows percentage of cells positive for binding of non-bnAbs 17B and 19B to 293F cell surface expressed BG505, BG505 F14/Vt8, BG505 F14, and BG505 Vt8 trimers in the presence and absence of sCD4 or CD4-Ig. FIG. 14G shows MFI data for binding of non-bnAbs 17B and 19B to 293F cell surface expressed BG505, BG505 F14/Vt8, BG505 F14, and BG505 Vt8 trimers in the presence and absence of sCD4 and CD4-Ig.

FIG. 15 shows BG505 SOSIP Env Mutations (HXB2 numbering). * Design DS-SOSIP.4mut from Chuang. G.-Y. et al. Structure-Based Design of a Soluble Prefusion-Closed HIV-1 Env Trimer with Reduced CD4 Affinity and Improved Immunogenicity. Journal of Virology 91, doi:10.1128/JVI.02268-16 (2017).

FIG. 16 shows CD4 Binding Kinetics and Affinities.

FIG. 17 shows bnAb Binding Affinities (K_{d} [nM]).

FIG. 18 shows Cryo-EM Data Collection and Refinement Statistics.

FIG. 19 shows Cell Surface Expressed Trimer—Percentage of Cells Binding.

FIG. 20 shows Cell Surface Expressed Trimer—MFI.

FIG. 21 shows smFRET Statistics.

FIG. 22A shows negative stain electron microscopy 2-dimensional class averages of the CH848.10.17 DT F14/Vt8 SOSIP trimer demonstrating the formation of intact trimer. FIG. 22B shows negative stain electron microscopy 2-dimensional class averages of the CH505 M5 G458Y F14 GNTI-SOSIP trimer demonstrating the formation of intact trimer.

FIG. 23A shows surface plasmon resonance response values for CH848.10.17 DT F14/Vt8 SOSIP, CH848.10.17 DT DS SOSIP and CH848.10.17 DT 4.1 SOSIP trimers comparing the interaction of each with the open state, bridging sheet targeting 17B antibody. FIG. 23B shows surface plasmon resonance response values for CH848.10.17 DT F14/Vt8 SOSIP, CH848.10.17 DT DS SOSIP and CH848.10.17 DT 4.1 SOSIP trimers in the presence of saturating, open state triggering sCD4 comparing the interaction of each with the open state, bridging sheet targeting 17B antibody.

FIG. 24A shows surface plasmon resonance response values for CH848.10.17 DT F14/Vt8 SOSIP, CH848.10.17 DT DS SOSIP and CH848.10.17 DT 4.1 SOSIP trimers comparing the interaction of each with the open state, variable region 3 (V3) targeting 19B antibody. FIG. 24B shows surface plasmon resonance response values for CH848.10.17 DT F14/Vt8 SOSIP, CH848.10.17 DT DS SOSIP and CH848.10.17 DT 4.1 SOSIP trimers in the presence of saturating, open state triggering sCD4 comparing the interaction of each with the open state, variable region 3 (V3) targeting 19B antibody.

FIG. 25A shows Surface plasmon resonance response values for CH505 M5 G458Y F14 GNTI-SOSIP and CH505 M5 G458Y 4.1 GNTI-SOSIP trimers comparing the interaction of each with the open state, bridging sheet targeting 17B antibody. FIG. 25B shows B surface plasmon resonance response values CH505 M5 G458Y F14 GNTI-SOSIP and CH505 M5 G458Y 4.1 GNTI-SOSIP trimers in the presence of saturating, open state triggering sCD4 comparing the interaction of each with the open state, bridging sheet targeting 17B antibody.

FIG. 26A shows surface plasmon resonance response values for CH505 M5 G458Y F14 GNTI-SOSIP and CH505 M5 G458Y 4.1 GNTI-SOSIP trimers comparing the interaction of each with the open state, variable region 3 (V3) targeting 19B antibody. FIG. 26B shows surface plasmon resonance response values for CH505 M5 G458Y F14 GNTI-SOSIP and CH505 M5 G458Y 4.1 GNTI-SOSIP trimers in the presence of saturating, open state triggering sCD4 comparing the interaction of each with the open state, variable region 3 (V3) targeting 19B antibody.

FIGS. 27A-27C show Effects of sCD4 and BMS on V3 binding. FIG. 27A surface plasmon resonance responses for BG505 F14/Vt8 SOSIP trimer interacting with V3 targeting 3074 antibody unliganded (red), in the presence of sCD4 (blue), and in the presence of both sCD4 and the small molecule, open state inhibiting BMS-626529 (green). FIG. 27B shows surface plasmon resonance responses for BG505 F14/Vt8 SOSIP trimer interacting with V3 targeting 447-52D antibody unliganded (red), in the presence of sCD4 (blue), and in the presence of both sCD4 and the small molecule, open state inhibiting BMS-626529 (green). FIG. 27C shows surface plasmon resonance responses for BG505 F14/Vt8 SOSIP trimer interacting with V3 targeting F39F antibody unliganded (red), in the presence of sCD4 (blue), and in the presence of both sCD4 and the small molecule, open state inhibiting BMS-626529 (green).

FIGS. 28A-28C show Effects of sCD4 and BMS on V3 binding. FIG. 28A show surface plasmon resonance responses for BG505 F14 SOSIP trimer interacting with V3 targeting 3074 antibody unliganded (red), in the presence of sCD4 (blue), and in the presence of both sCD4 and the small molecule, open state inhibiting BMS-626529 (green). FIG. 28B shows surface plasmon resonance responses for BG505 F14 SOSIP trimer interacting with V3 targeting 447-52D antibody unliganded (red), in the presence of sCD4 (blue), and in the presence of both sCD4 and the small molecule, open state inhibiting BMS-626529 (green). FIG. 28C shows surface plasmon resonance responses for BG505 F14 SOSIP trimer interacting with V3 targeting F39F antibody unliganded (red), in the presence of sCD4 (blue), and in the presence of both sCD4 and the small molecule, open state inhibiting BMS-626529 (green).

FIGS. 29A-29C shows Effects of sCD4 and BMS on V3 binding. FIG. 29A shows surface plasmon resonance responses for BG505 Vt8 SOSIP trimer interacting with V3 targeting 3074 antibody unliganded (red), in the presence of sCD4 (blue), and in the presence of both sCD4 and the small molecule, open state inhibiting BMS-626529 (green). FIG. 29B shows surface plasmon resonance responses for BG505 Vt8 SOSIP trimer interacting with V3 targeting 447-52D antibody unliganded (red), in the presence of sCD4 (blue), and in the presence of both sCD4 and the small molecule, open state inhibiting BMS-626529 (green). FIG. 29C shows surface plasmon resonance responses for BG505 Vt8 SOSIP trimer interacting with V3 targeting F39F antibody unliganded (red), in the presence of sCD4 (blue), and in the presence of both sCD4 and the small molecule, open state inhibiting BMS-626529 (green).

FIGS. 30A-30C shows Effects of sCD4 and BMS on V3 binding. FIG. 30A shows surface plasmon resonance responses for BG505 SOSIP trimer interacting with V3 targeting 3074 antibody unliganded (red), in the presence of sCD4 (blue), and in the presence of both sCD4 and the small molecule, open state inhibiting BMS-626529 (green). FIG. 30B shows surface plasmon resonance responses for BG505 SOSIP trimer interacting with V3 targeting 447-52D antibody unliganded (red), in the presence of sCD4 (blue), and in the presence of both sCD4 and the small molecule, open state inhibiting BMS-626529 (green). FIG. 30C shows surface plasmon resonance responses for BG505 SOSIP trimer interacting with V3 targeting F39F antibody unliganded (red), in the presence of sCD4 (blue), and in the presence of both sCD4 and the small molecule, open state inhibiting BMS-626529 (green).

FIGS. 31A-D show percentage of full length Env gp160 HEK293 cells testing positive for interaction with antibodies Ab82 (control), open state, bridging sheet targeting 17B, V3 open state targeting 19B, and 7B2 in the absence and presence of sCD4, eCD4-Ig, or CD4-IgG2. FIG. 31A shows CH848 DT gp160. FIG. 31B shows CH848 F14 DT gp160. FIG. 31C shows CH848 Vt8 DT gp160. FIG. 31D shows CH848 DT F14/Vt8 gp160. The results indicate that, while the CH848 DT gp160 open state is triggered, the stabilized Envs are not.

FIGS. 32A-D show percentage of full length Env gp160 HEK293 cells testing positive for interaction with antibodies Ab82 (control), trimer apex targeting, quaternary specific PGT145, and V2 targeting CH01 and PG9L in the absence and presence of sCD4, eCD4-Ig, or CD4-IgG2. FIG. 32A shows CH848 DT gp160. FIG. 32B shows CH848 F14 DT gp160. FIG. 32C shows CH848 Vt8 DT gp160. FIG. 32D shows CH848 DT F14/Vt8 gp160. The results indicate that the F14 stabilization most closely recapitulates the WT CH848 DT gp160 Env antigenically.

FIGS. 33A-D show Percentage of full length Env gp160 HEK293 cells testing positive for interaction with antibodies Ab82 (control) and V3-glycan targeting antibodies PGT125, DH270 UCA3, and DH270 UCA4 in the absence and presence of sCD4, eCD4-Ig, or CD4-IgG2. FIG. 33A shows CH848 DT gp160. FIG. 33B shows CH848 F14 DT gp160. FIG. 33C shows CH848 Vt8 DT gp160. FIG. 33D shows CH848 DT F14/Vt8 gp160. Results indicate that the F14 construct displays an interaction profile consistent with the wild type CH848 DT gp160.

FIG. 34 shows BMS626529 Effect on Mutant Binding with VRC01. Surface plasmon resonance experiments of stabilized and non-stabilized BG505 and CH848 SOSIP trimers interacting with CD4 binding site targeting broadly neutralizing antibody VRC01 in the presence and absence of small molecule, open state inhibiting BMS-626529. With the exception of CH848 F14/Vt8 DT, the presence of BMS-626529 reduces the binding of VRC01.

FIG. 35 shows BMS626529 Bound and Unbound CH848 F14/Vt8 Interactions. Surface plasmon resonance experiments of the F14/Vt8 stabilized CH848 SOSIP trimer interacting with bnAbs VRC01, CH31, B12, CH01, and the CH01 unmutated common ancestor antibodies in the presence and absence of small molecule, open state inhibiting BMS-626529. The results indicate that the BMS-626529 molecule enhances the interaction between the SOSIP and antibodies.

FIGS. 36A-D show Domain organization and immunogenicity of the BG505 (Rabbit study R41) and BG505 F14 (Rabbit study R47) SOSIP. FIG. 36A shows serum neutralization results with standard deviation for BG505 WT and F14 SOSIP immunized rabbits for BG505, SF162.LS, and MW965.26 virus. FIG. 36B shows cartoon representation of the VRC34 blocking experiment. FIG. 36C shows VRC34 serum blocking ELISA results for BG505 WT SOSIP immunized rabbits with SOSIP captured by either PGT151 (red) or PGT145 (blue). FIG. 36D shows VRC34 serum blocking ELISA results for BG505 F14 SOSIP immunized rabbits with SOSIP captured by either PGT151 (red) or PGT145 (blue).

FIG. 37 shows VRC34 ELISA binding to PGT145 and PGT151 capture BG505 F14 SOSIP. Interaction of VRC34 measured at increasing concentration. Curve fit using one site specific interaction model.

FIG. 38 shows neutralization data from Rabbit study R41.

FIG. 39 shows neutralization data from Rabbit study R47.

FIG. 40 shows neutralization data from Rabbit study R48

FIG. 41A-J show sequences of envelopes of the technology. Italicized amino acids at the N-terminus of the sequence show the signal peptide.

FIG. 42 shows an amino acid sequence alignment of envelopes CH0848.D0949.10.17_N133D_N138T.ch.SOSIP (italicized amino acids at the N-terminus of the sequence show the signal peptide), CH0848.D0949.10.17_N133D_N138T.ch.SOSIP.F14, and CH0848.D0949.10.17_N133D_N138T.ch.SOSIP.Vt8. F14 and Vt8 changes are indicated.

FIG. 43 shows an amino acid sequence alignment of envelopes CH0848.D0949.10.17_N133D_N138T.SOSIP (Italicized amino acids at the N-terminus of the sequence show the signal peptide); CH0848.D0949.10.17_N133D_N138T.SOSIP.F14; CH0848.D0949.10.17_N133D_N138T.SOSIP.F14.Vt8:CH0848.D0949.10.17_N133D_N138T.SOSIP.Vt8. F14 and Vt8 changes are indicated

FIG. 44 shows a map of the positions of F14 and Vt8 mutations in the CH0848.D0949.10.17_N133D_N138T.SOSIP sequence.

FIGS. 45A-E show various sequences of envelopes of the technology. Italicized amino acids at the N-terminus of the sequence show the signal peptide.

FIGS. 46A-D show various sequences of envelopes of the technology. Amino acid sequences do not include the signal peptide.

FIGS. 47A-D show various sequences of envelopes of the technology. Amino acid sequences do not include the signal peptide.

FIG. 48 shows BG505gp160T332N_F14_VT8 amino acid sequence. Italicized amino acids at the N-terminus of the sequence show the signal peptide.

FIG. 49 shows amino acid sequence of CH505.M5gp160_G458Y_F14_VT8. Italicized amino acids at the N-terminus of the sequence show the signal peptide.

FIG. 50 shows amino acid sequence of CH0848.d949.10.17N133DN138Tgp160_F14_VT8. Italicized amino acids at the N-terminus of the sequence show the signal peptide.

FIG. 51 shows amino acid sequence of BG505gp140SOSIPT332N. Italicized amino acids at the N-terminus of the sequence show the signal peptide.

FIG. 52 shows amino acid sequence of HV1301509; CH0848.3.d1305.10.19gp160.

FIG. 53 shows amino acid sequence of HV1301580; CH848.3.D1305.10.19_D949V3.DS.SOSIP (19CV3).

FIG. 54 shows non-limiting embodiments of nucleic acid sequences of envelopes of the technology.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254:225-244, 2013). BnAbs are protective in rhesus macaques against SHIV challenge, but as yet, are not induced by current vaccines.

The technology provides methods of using these stabilized envelope immunogens.

In certain aspects, the technology provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect the technology provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

### mRNA

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the technology provides an expression vector comprising any of the nucleic acid sequences of the technology, wherein the nucleic acid is operably linked to a promoter. In certain aspects the technology provides an expression vector comprising a nucleic acid sequence encoding any of the polypeptides of the technology, wherein the nucleic acid is operably linked to a promoter. In certain embodiments, the nucleic acids are codon optimized for expression in a mammalian cell, in vivo or in vitro. In certain aspects the technology provides nucleic acids comprising any one of the nucleic acid sequences of technology. In certain aspects the technology provides nucleic acids consisting essentially of any one of the nucleic acid sequences of technology. In certain aspects the technology provides nucleic acids consisting of any one of the nucleic acid sequences of technology. In certain embodiments the nucleic acid of the technology, is operably linked to a promoter and is inserted in an expression vector. In certain aspects the technology provides an immunogenic composition comprising the expression vector.

In certain aspects the technology provides a composition comprising at least one of the nucleic acid sequences of the technology. In certain aspects the technology provides a composition comprising any one of the nucleic acid sequences of technology. In certain aspects the technology provides a composition comprising at least one nucleic acid sequence encoding any one of the polypeptides of the technology.

The envelope used in the compositions and methods of the technology can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

In certain embodiments the envelope is any of the forms of HIV-1 envelope. In certain embodiments the envelope is gp120, gp140, gp145 (i.e. with a transmembrane), gp150. In certain embodiments, gp140 designed to form a stable trimer and various non-limiting examples of sequences of stable trimers are known. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof”, See also WO/2017151801 each content is herein incorporated by reference in its entirety. In certain embodiments the envelopes of the technology are engineered and comprise non-naturally occurring modifications.

In certain embodiments, the envelope is in a liposome. In certain embodiments the envelope comprises a transmembrane domain with a cytoplasmic tail embedded in a liposome. In certain embodiments, the nucleic acid comprises a nucleic acid sequence which encodes a gp120, gp140, gp145, gp150, gp160.

In certain embodiments, where the nucleic acids are operably linked to a promoter and inserted in a vector, the vector is any suitable vector. Non-limiting examples include VSV, replicating rAdenovirus type 4. MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In certain aspects the technology provides a cell comprising a nucleic acid encoding any one of the envelopes of the technology suitable for recombinant expression. In certain aspects, the technology provides a clonally derived population of cells encoding any one of the envelopes of the technology suitable for recombinant expression. In certain aspects, the technology provides a sable pool of cells encoding any one of the envelopes of the technology suitable for recombinant expression.

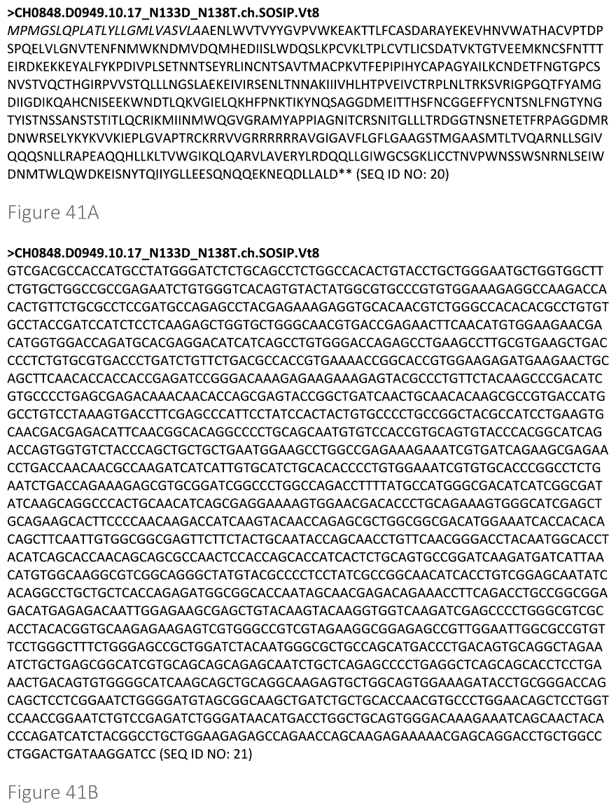

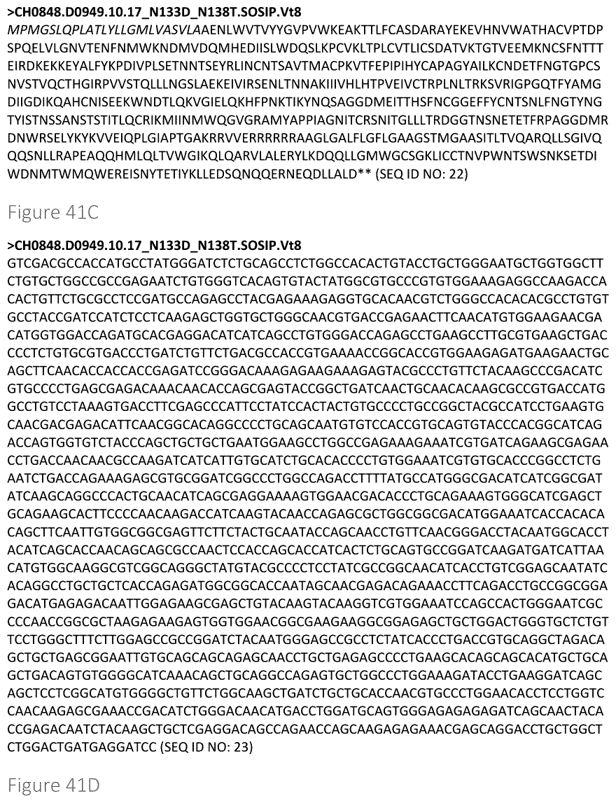

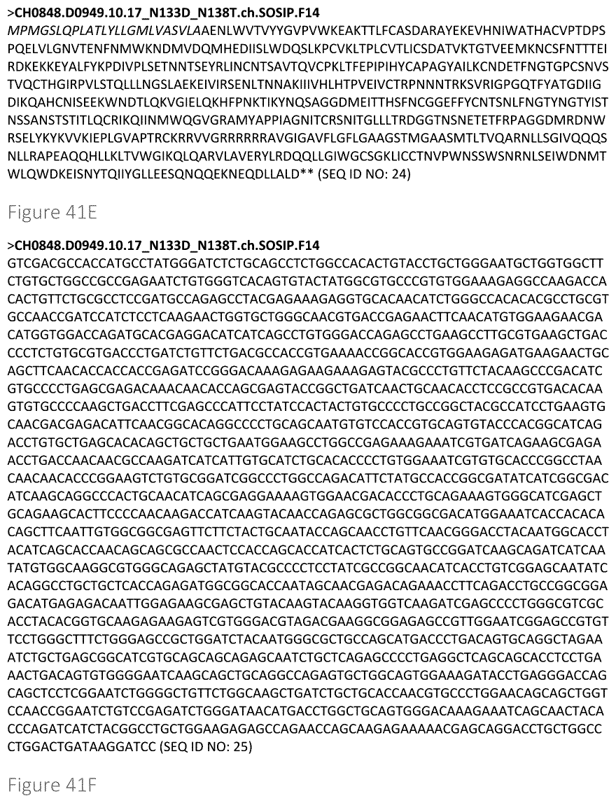

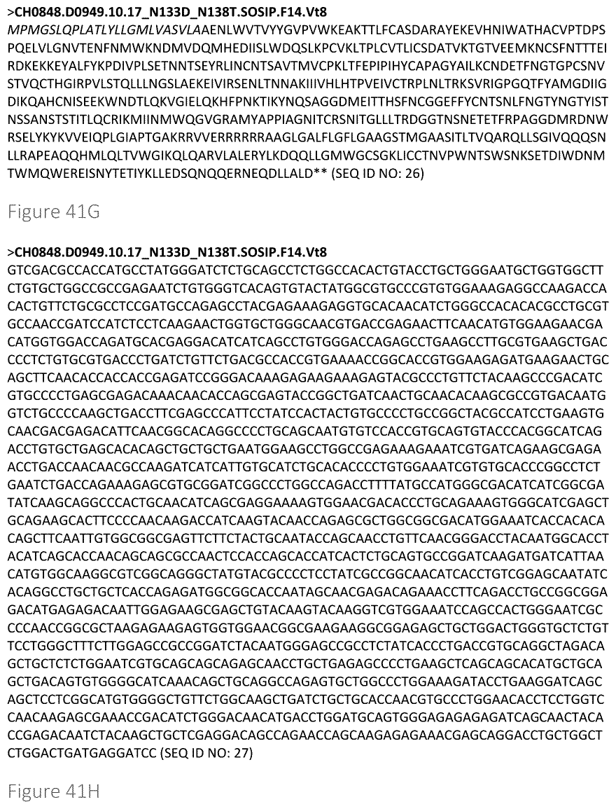

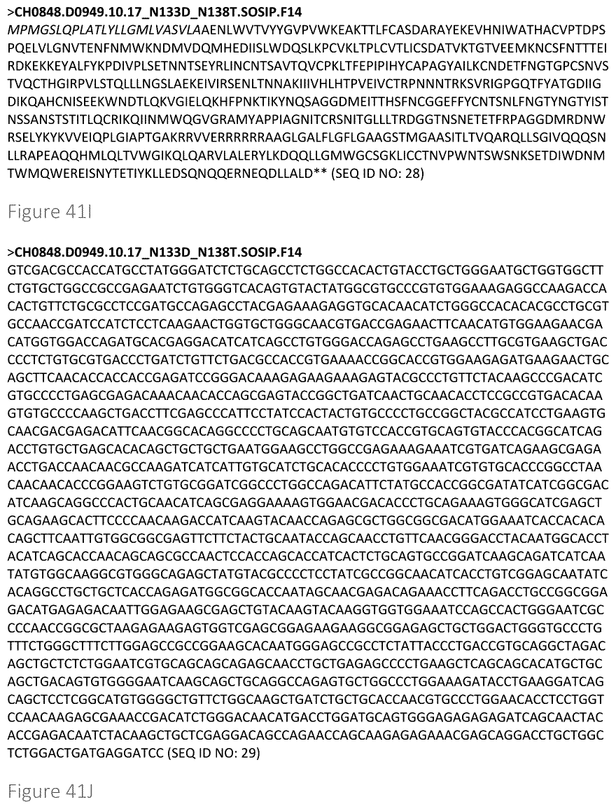

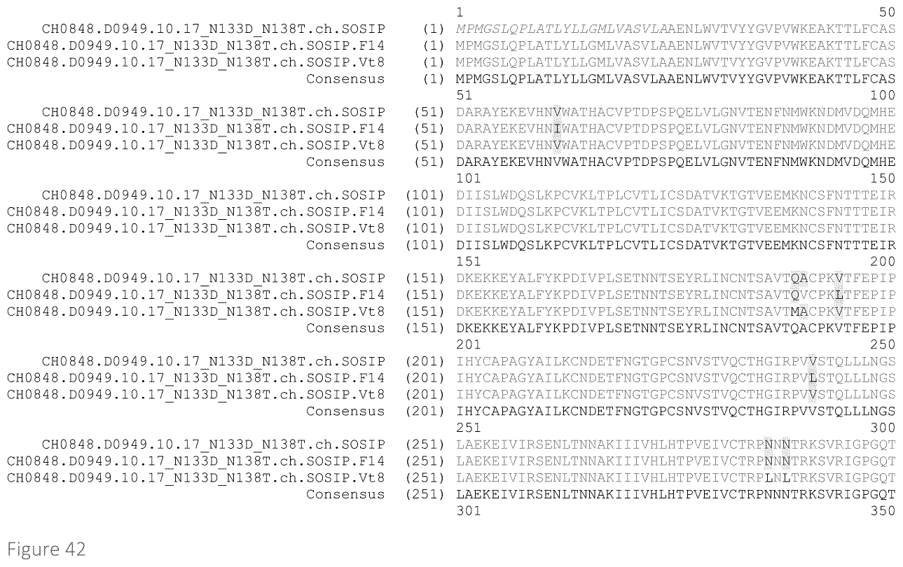

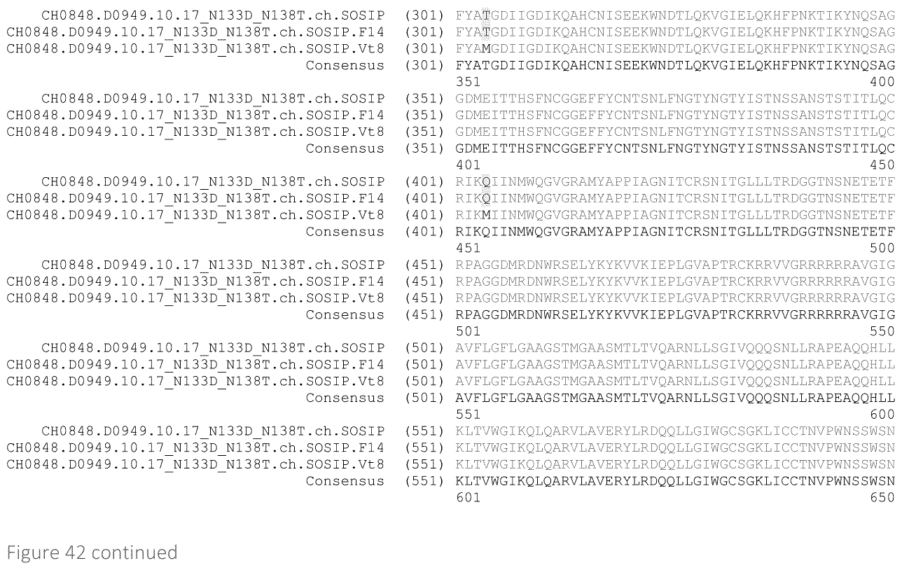

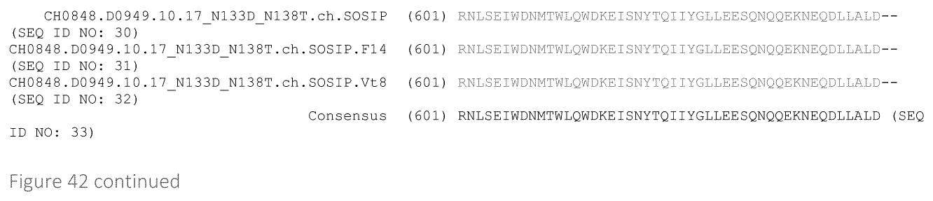

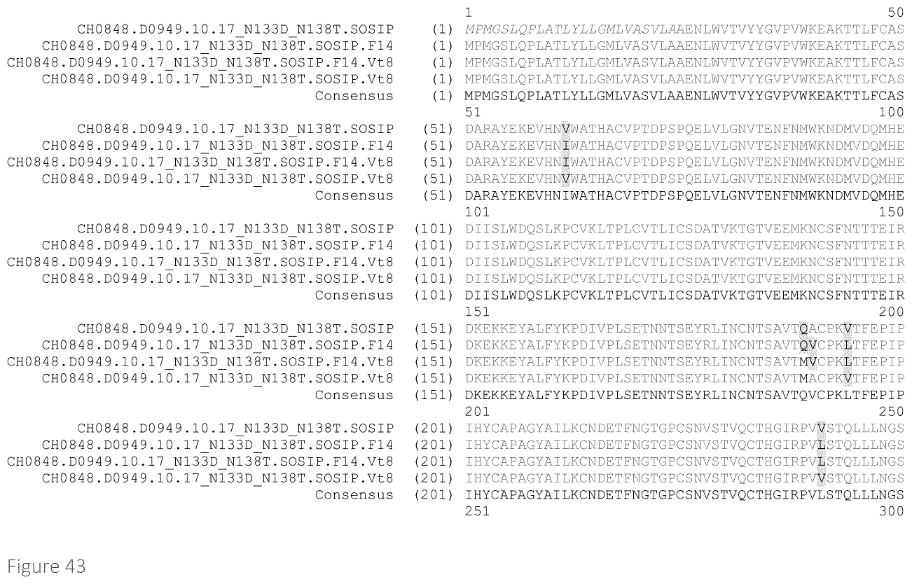

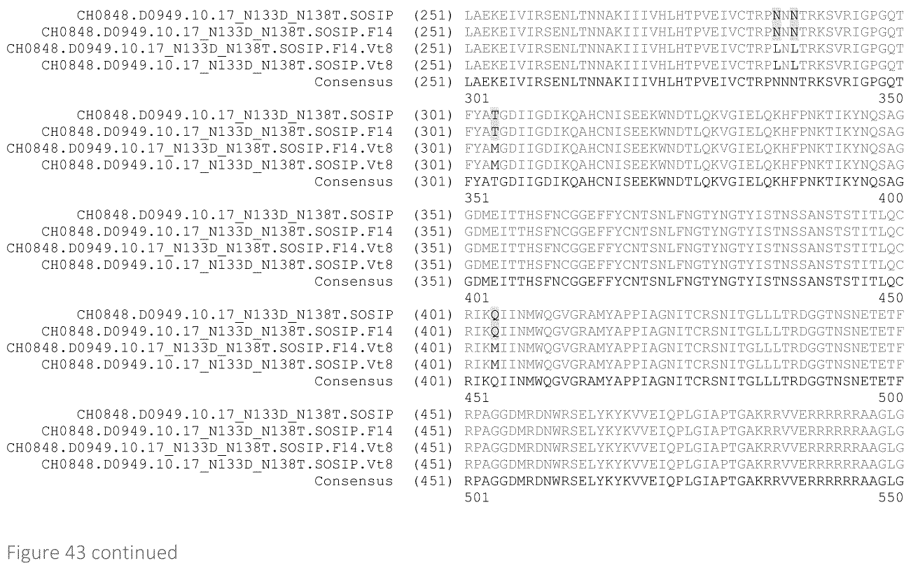

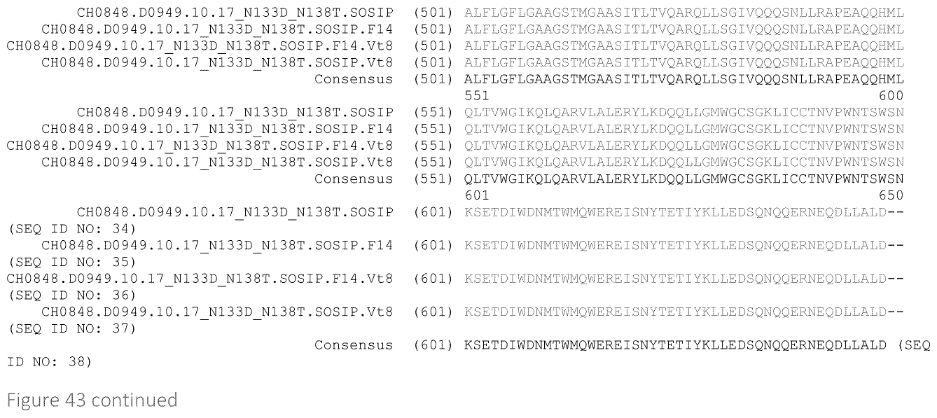

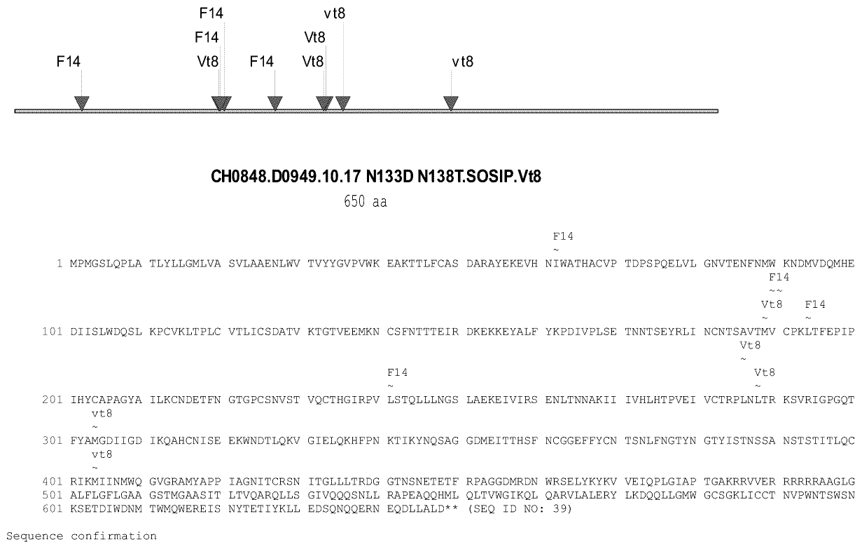

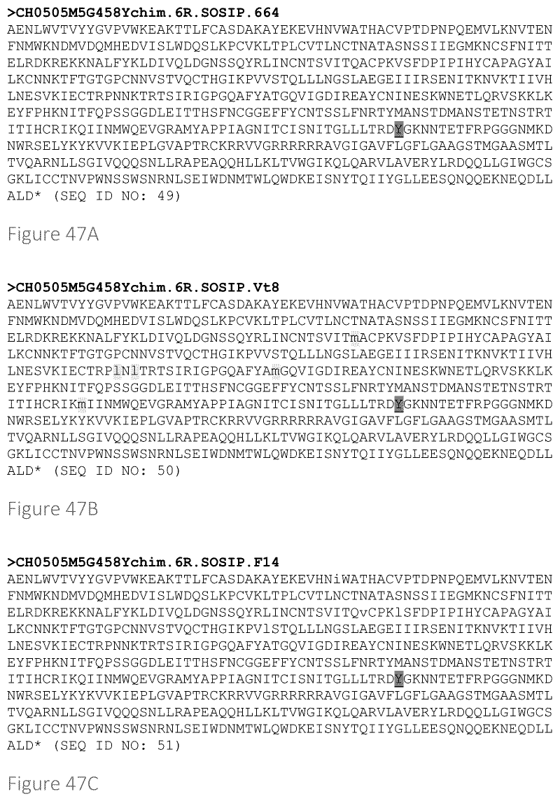

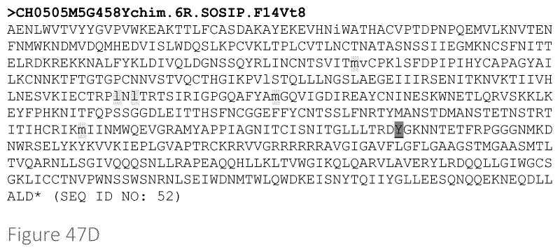

In certain aspects, the technology provides a recombinant HIV-1 envelope polypeptide as described here, wherein the polypeptide is a non-naturally occurring protomer designed to form an envelope trimer. The technology also provides nucleic acids encoding these recombinant polypeptides. Non-limiting examples of amino acids and nucleic acid of such protomers are shown in FIGS. 41-51.

In certain aspects the technology provides a recombinant trimer comprising three identical protomers of an envelope. In certain aspects the technology provides an immunogenic composition comprising the recombinant trimer and a carrier, wherein the trimer comprises three identical protomers of an HIV-1 envelope as described herein. In certain aspects the technology provides an immunogenic composition comprising nucleic acid encoding these recombinant HIV-1 envelope and a carrier.

### Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

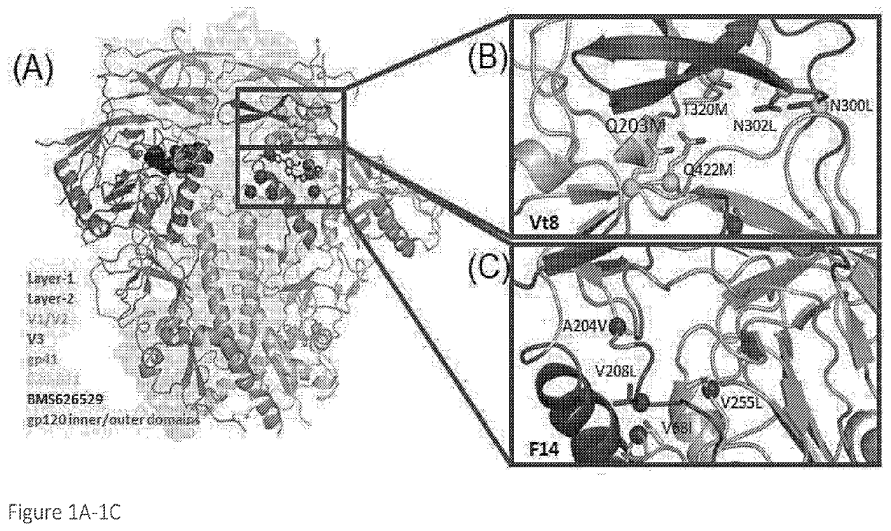

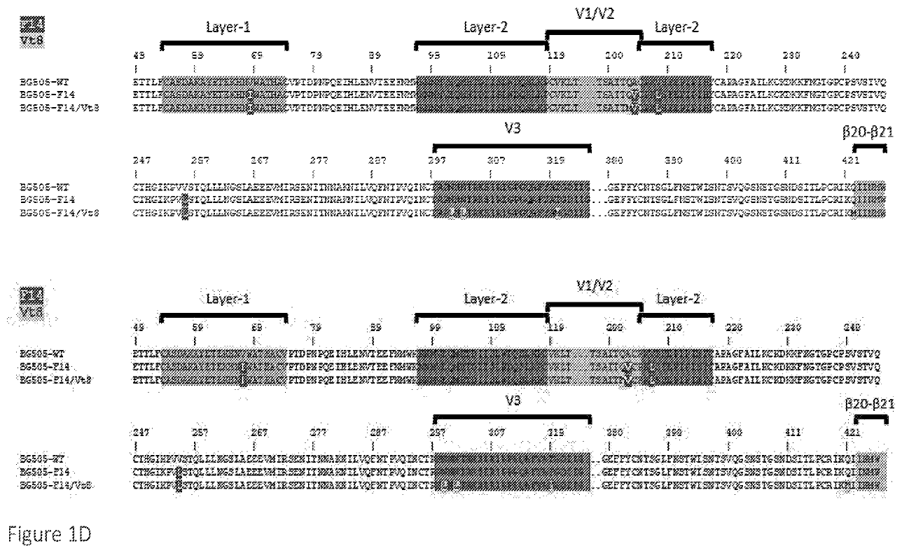

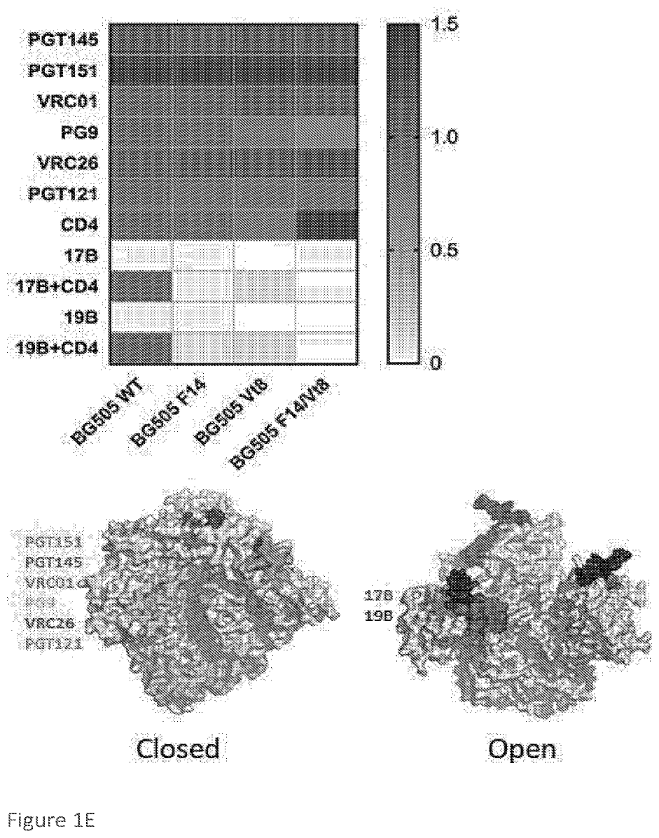

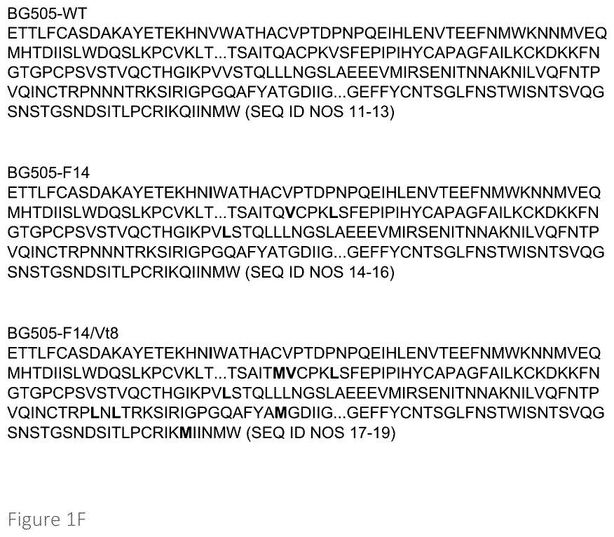

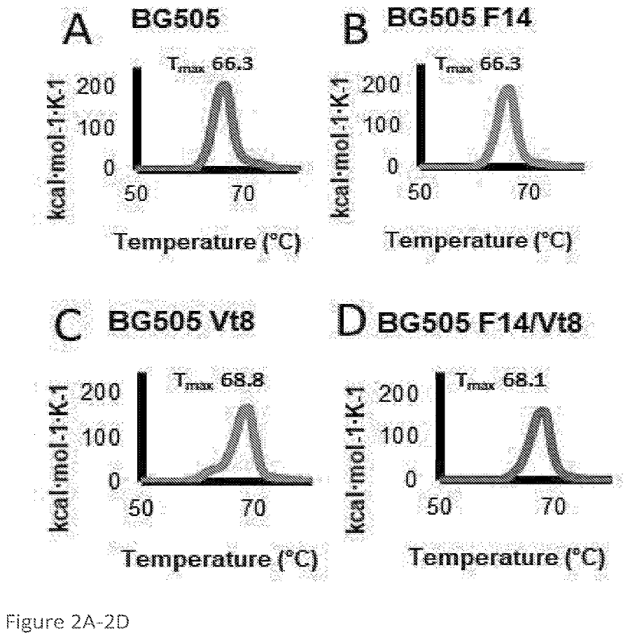

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR (SEQ ID NO: 1).” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. Apr; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR (SEQ ID NO: 2) is changed to ERVVEREKE (SEQ ID NO: 3), and is one example of an uncleaved gp140 form. Another example is the gp140C form which has the REKR (SEQ ID NO: 1) site changed to SEKS (SEQ ID NO: 4). See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present technology involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, X can be any amino acid) and “ VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (SEQ ID NO: 5) (rest of envelope sequence is indicated as “ . . . ”). In other embodiments, the delta N-design described for CH505 T/F envelope can be used to make delta N-designs of other CH505 envelopes. In certain embodiments, the technology relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the technology provides composition and methods which use a selection of sequential Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit immune response. Envs as proteins would be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the technology provides compositions and methods of Env genetic immunization either alone or with Envy proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the technology contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLOS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized. for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLOS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the technology contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by incellart.

### mRNA

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain aspects the technology contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples the leaders sequence is human Tissue Plasminogen Activator (TPA) sequence, human CD5 leader sequence (e.g. MPMGSLQPLATLYLLGMLVASVLA (SEQ ID NO: 6). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen [Leroux-Roels et al., IABS Conference, April 2013]. In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355|Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

### Multimeric Envelopes

Presentation of antigens as particulates reduces the B cell receptor affinity necessary for signal transduction and expansion (See Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axis the envelope protein is fused. Therefore the assembly of the three-fold axis also clusters three HIV-1 envelope protomers together to form an envelope trimer. Each ferritin particle has 8 axises which equates to 8 trimers being displayed per particle. See e.g. Sliepen et al. Retrovirology 201512:82, DOI: 10.1186/s12977-015-0210-4; See also FIG. 24H-J.

Another approach to multimerize expression constructs uses staphylococcus Sortase A transpeptidase ligation to conjugate inventive envelope trimers to cholesterol. The trimers can then be embedded into liposomes via the conjugated cholesterol. To conjugate the trimer to cholesterol either a C-teminal LPXTG tag (SEQ ID NO: 7) or a N-terminal pentaglycine repeat tag (SEQ ID NO: 8) is added to the CH505 envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles.

The technology provides design of envelopes and trimer designs wherein the envelope comprises a linker which permits addition of a lipid, such as but not limited to cholesterol, via a Sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. doi:10.1002/cbic.200800724; Proft, T. Sortase-mediated protein ligation: an emerging biotechnology tool for protein modification and immobilisation. Biotechnol Lett (2010) 32:1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

Non-limiting embodiments of envelope designs for use in Sortase A reaction are shown in FIG. 24 B-D in WO2017151801, incorporated by reference in its entirety.

Additional sortase linkers could be used so long as their position allows multimerization of the envelopes.

Table 1 shows a summary of sequences. In non-limiting embodiments any of the sequences and designs disclosed in WO2014/042669, WO/2017151801, WO/2017152146 and WO/2018161049, which contents are incorporated by reference their entirety, could comprise additional stabilizing mutations, combinations and/or subsets of mutations described herein.

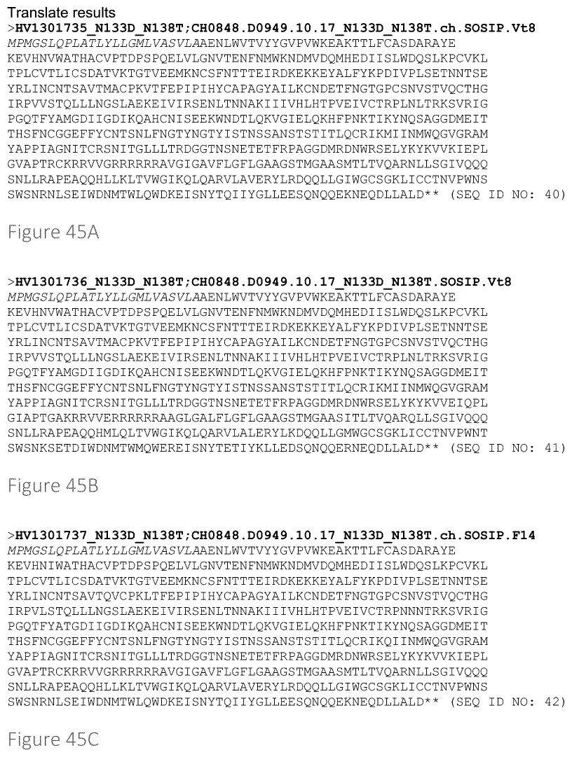

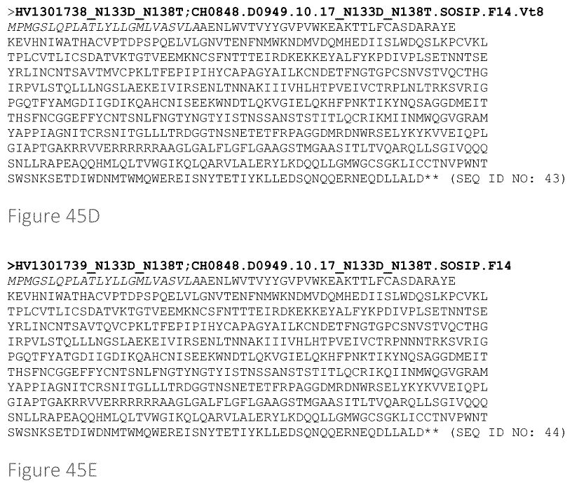

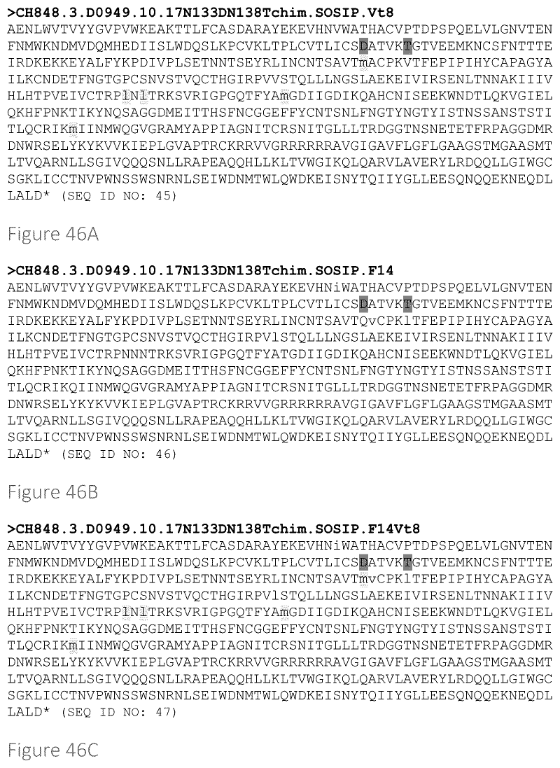

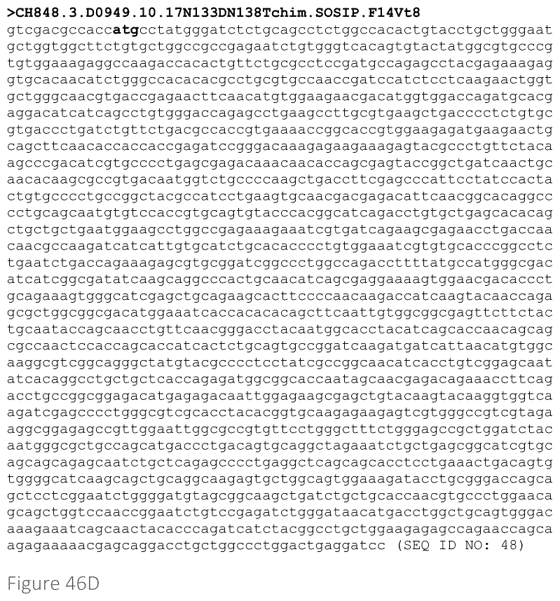

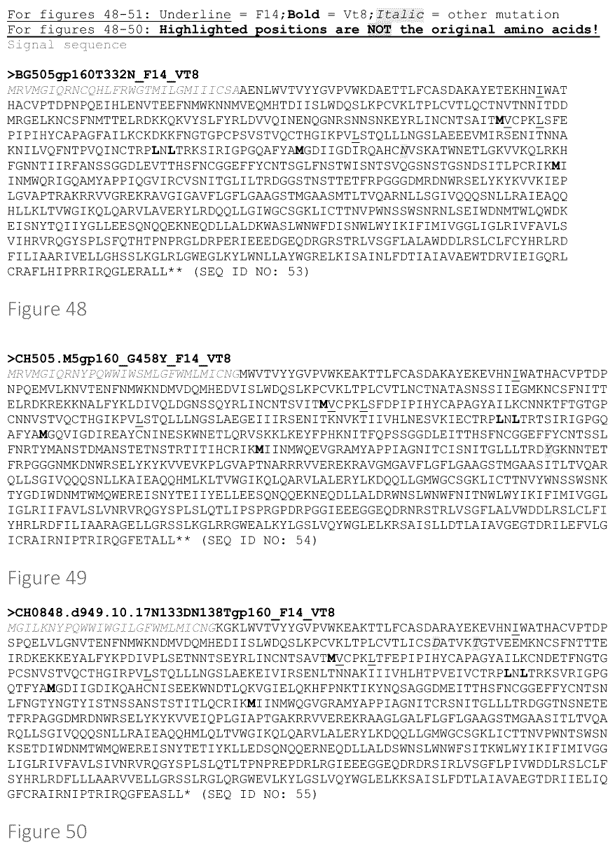

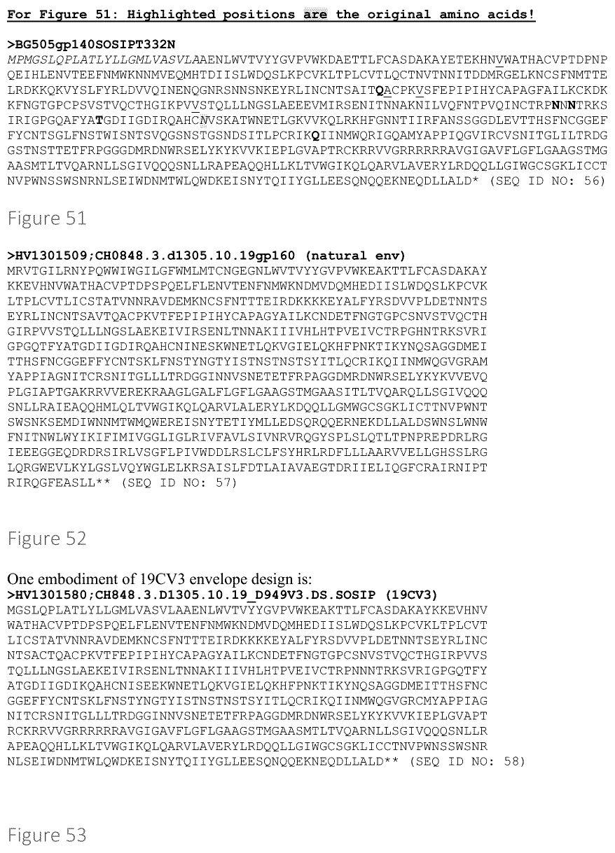

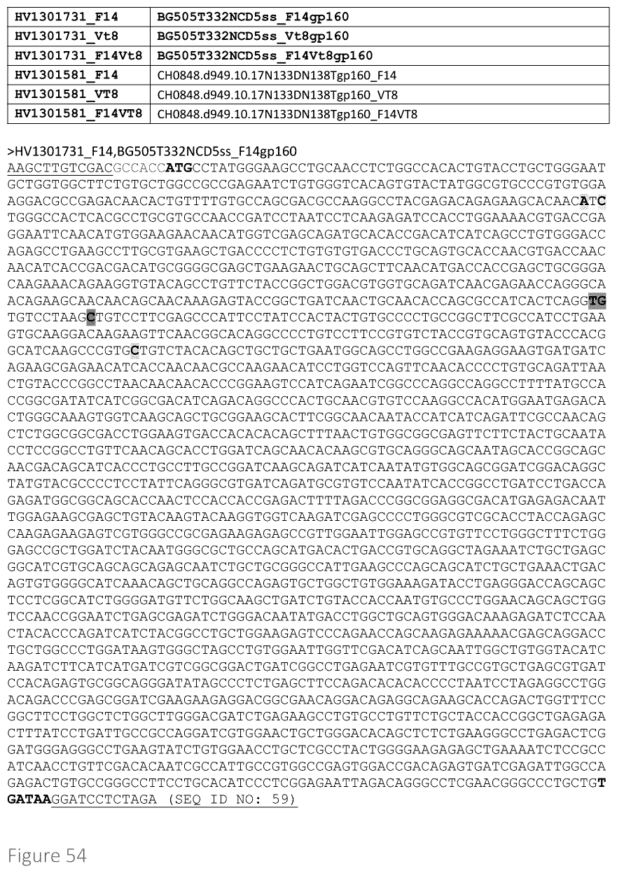

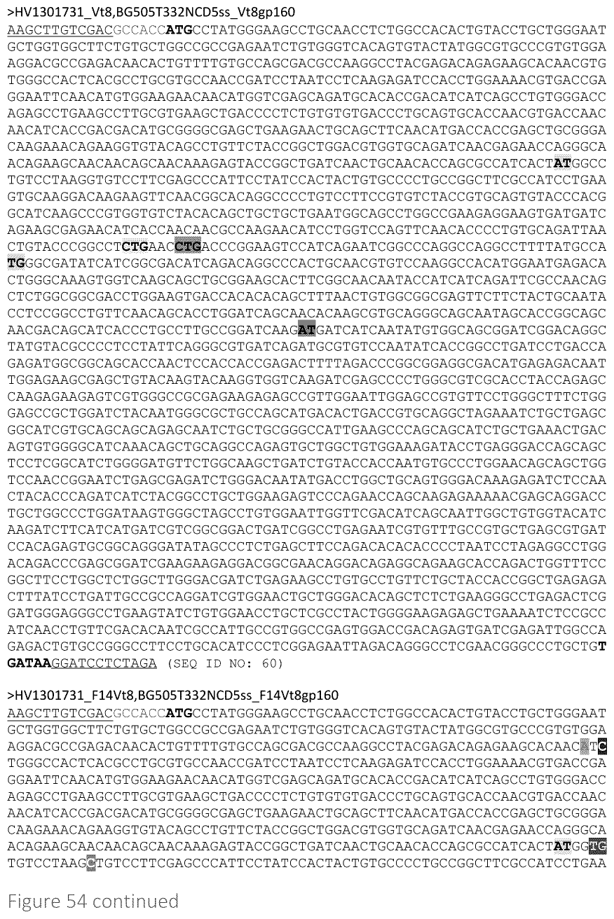

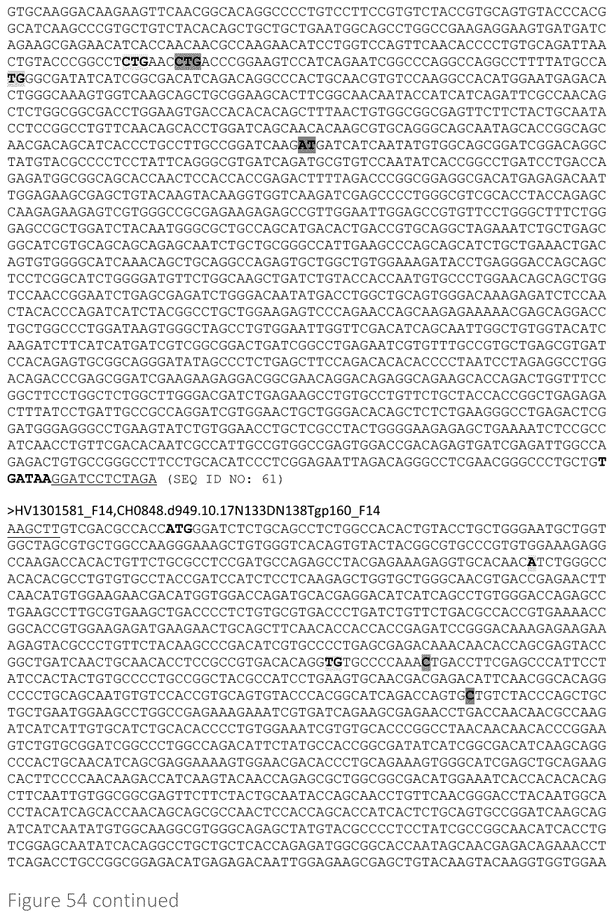

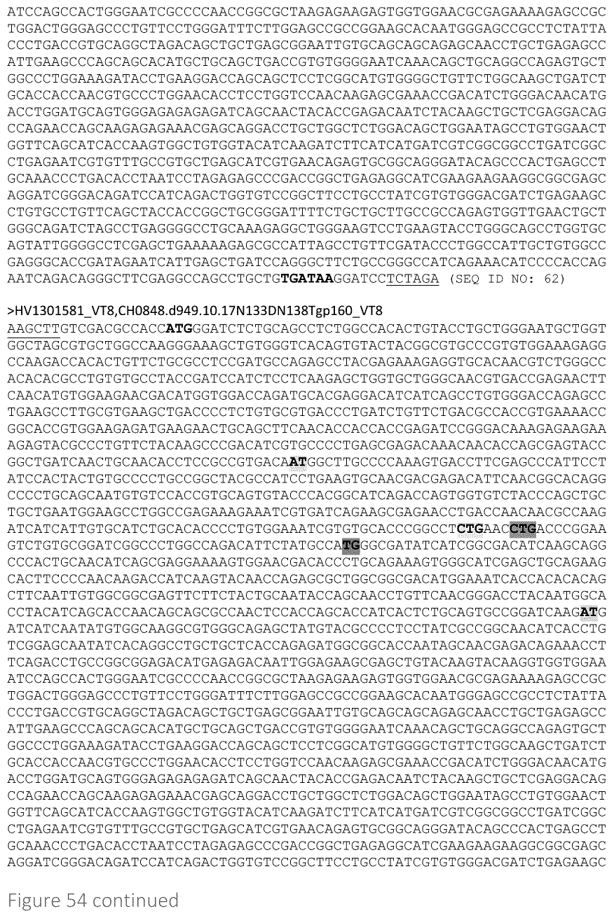

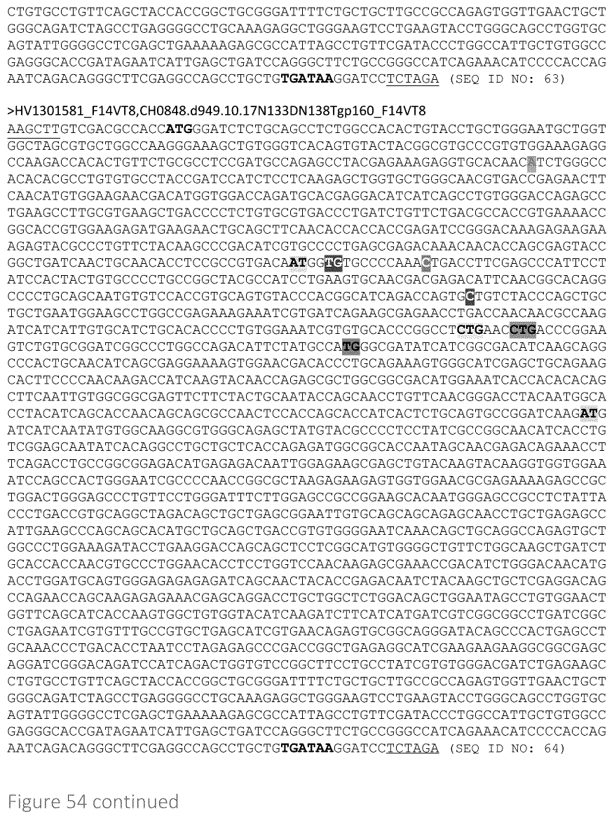

TABLE 1

| | Amino | | |
|---|---|---|---|
| | acid or | | |
| Name | Nucleic | Design | FIG./note |
| CH0848.D0949.10.17_N133D_N138T | | | |
| >CH0848.D0949.10.17_N133D_N138T.ch.SOSIP.Vt8 | Both | ch.SOSIP.Vt8 | FIG. 41A |
| | | | and 41B |
| | | | FIG. 45A |
| | | | 46A |
| >CH0848.D0949.10.17_N133D_N138T.SOSIP.Vt8 | Both | SOSIP.Vt8 | FIG. 41C |
| | | | and D |
| | | | FIG. 45B |
| >CH0848.D0949.10.17_N133D_N138T.ch.SOSIP.F14 | Both | ch.SOSIP.F14 | FIG. 41E |
| | | | and F |
| | | | FIG. 45C |
| | | | 46B |
| >CH0848.D0949.10.17_N133D_N138T.SOSIP.F14 | both | SOSIP.F14 | FIG. 41I |
| | | | and J |
| | | | FIG. 45E |
| >CH848.3.D0949.10.17N133DN138Tchim.SOSIP.F14Vt8 | aa | chim.SOSIP.F14 | FIG. 46C |
| | nt | Vt8 | FIG. 46D |
| >CH0848.D0949.10.17_N133D_N138T.SOSIP.F14.Vt8 | both | SOSIP.F14.Vt8 | FIG. 41G |
| | | | and H |
| | | | FIG. 45D |
| >CH0848.d949.10.17N133DN138Tgp160_F14_VT8 | aa | gp160_F14_VT8 | FIG. 50 |
| For additional non-limiting embodiments of CH848 sequences | | | |
| also WO/2017152146 and WO/2018161049 | | | |
| BG505 | | | |
| BG505gp160T332N_F14_VT8 | aa | | FIG. 48 |
| >BG505gp140SOSIPT332N | aa | | FIG. 51 |
| 19CV3 | | | |
| HV1301580; CH848.3.D1305.10.19_D949V3.DS.SOSIP | | | FIG. 53 |
| (19CV3) | | | |
| CH505 | | | |
| CH505 sequences (non-limiting embodiments) | | | |
| T/F, M5, M6, M11 and selections disclosed in | | | |
| WO2014/042669 | | | |
| Sequences and selections disclosed in WO2017/151801 | | | |
| G458Mut | | | |
| >CH505.M5gp160_G458Y_F14_VT8 | aa | | 49 |
| >CH0505M5G458Ychim.6R.SOSIP.664 | aa | | 47A |
| | | | (G458Y |
| | | | position is |
| | | | underlined) |
| >CH0505M5G458Ychim.6R.SOSIP.Vt8 | aa | Vt8 | 47B |
| | | | (G458Y |
| | | | position is |
| | | | underlined) |
| >CH0505M5G458Ychim.6R.SOSIP.F14 | aa | F14 | 47C |
| | | | (G458Y |
| | | | position is |
| | | | underlined) |
| >CH0505M5G458Ychim.6R.SOSIP.F14Vt8 | aa | F14Vt8 | 47D |
| | | | (G458Y |
| | | | position is |
| | | | underlined) |

Any of the designs could be made chimeric (including gp41 from BG505) and/or non-chimeric. Any of the designs described herein could also be designed to exclude SOSIP mutations.

### EXAMPLE 1

Allosteric control of Env conformational states. In order to design a non-covalently stabilized soluble SOSIP and membrane bound Env, we first examined the BMS-626529 stabilization of the Env SOSIP. The BMS-626529 compound prevents transitions from the prefusion closed state to the open state and blocks CD4 interaction, therefore eliminating exposure of non-neutralizing antibody epitope exposure. Further, BMS-626529 is known to stabilize state-1, a major target in Env immunogen design efforts, in the membrane bound Env. We reasoned that an understanding of BMS-626529 stabilization could lead to novel methods by which to stabilize both the soluble SOSIP and membrane bound Envs in a more native, closed state. A recent structure of a BG505 SOSIP in complex with BMS-626529 revealed the compound resides in an induced pocket below the β20-β21 loop between layers 1 & 2 and the V1/V2 apex. The BMS compound likely interrupts β20-β21 loop rearrangements important for allosteric induction of closed to open state transitions. To facilitate rational design based upon these features of the BMS-626529 binding, we aimed to develop a theoretical mechanism for rearrangements in the gp120 allosteric network that allow for CD4 triggering of the Env beginning from the β20-β21 loop. Residues in the β20-β21 loop important for Env triggering were recently identified. In particular, I423 was demonstrated to play an important role along with a previously identified V1/V2 residue, L193, in close proximity. Based upon this information as well as information indicating V3 move independently of V1/V2, we hypothesized that β20-β21 loop rearrangement induces V1/V2 dissociation from the trimer apex, therefore eliminating gp120 apex contacts. Concomitant formation of the bridging sheet would then communicate apex opening to layer-2. Transitions in layer-2 could then disrupt the layer-1 contacts. As layer-1 forms contacts with both layer-2 and the gp41 three-helix bundle, destabilization of layer-1 to layer-2 contacts would in turn destabilize layer-1 to gp41 contacts, thus reducing the barrier for gp120 rotation away from gp41. Together, this mechanism identifies a line of communication between the distant β20-β21 loop and the gp120 to gp41 three-helix bundle interface. Further, this mechanism identifies multiple sites as capable of controlling the Env conformational ensemble.

Design of conformationally stabilized Env constructs. Based upon the BMS-626529 interaction site and the theoretical mechanism for conformational control of the HIV-1 Env, we produced a set of BG505 Env mutations meant to capture the conformational effects of the BMS compound. Specifically, we generated a set of space-filling, hydrophobic mutations in the residues in contact with BMS-626529 in the SOSIP bound crystal structure. In combination with these mutations, mutations were made in layers 1 and 2 in order to prevent transitions in these regions. As V3 exposure occurs independently of V1/V2 exposure, we generated a set of hydrophobic mutations in the V1/V2-V3 interface in order to block V3 exposure. From these large sets of mutations, smaller sets of mutations were prepared in order to examine the effect of particular mutations and to maximize the likelihood of identifying a suitable set of mutations for downstream processing (Table 1). The BMS-626529 mimicking and layers 1 and 2 locking mutations were termed the ‘F’ series while the V3 locking mutations were termed the ‘Vt’ series. Each mutant was screened via transfection supernatant biolayer interferometry (BLI) in triplicate using, VRC01, PGT145, 17B, and 19B in order to examine gp120 folding, trimer formation, CD4i epitope exposure, and V3 exposure, respectively. Comparison of these results with the differences observed in the same assay for BG505 in the presence and absence of BMS-626529 identified ‘F’ series mutants F14 and F15 and ‘Vt’ series Vt-8 as suitable for downstream characterization. The antigenicity profiles of the F14 and F15 mutants did not differ. As F15 is identical to F14 except for one additional mutation, N337L, F14 was used for downstream analysis. This data suggests further exploration of the particular contribution of each mutation in the F14 stabilization of the trimer is warranted, as a further reduced set may confer a similar phenotype. Similarly, Vt8 itself may require only a subset of the mutations it contains.

The full set of tested mutations are listed below (HXB2 numbering) with a brief explanation for each. All were originally placed in BG505 SOSIP. They were then screened via BLI of small scale transfection supernatants. From the BLI data F14, F15 and Vt8 were expressed, purified, and screened for CD4 binding and triggering.

These sets of mutations were then put into CH848 10.17 DT and CH505 M5 SOSIP (F14, Vt8, and F14+Vt8) in addition to a BG505 SOSIP F14+Vt8.

### Full Set->Pack the BMS-626529 Binding Site and Lock the Layers in Place

The set of mutations referred to as F1 are V68I, S115V, A204L, V208L, V255W, N377L, M426W, M434W, and H66S.

Elimination* of N377L, M426W, and M434W may avoid over-packing the area. N377 may be important for folding as it is not totally buried. “Elimination” means that an F2 construct includes all F1 mutations except N337L, M426W, and M434W.

The set of mutations referred to as F2 are: V68I, S115V, A204L, V208L, V255W, and H66S

Elimination of S115V may be done if adding a V may be too large for the area where S115 resides.

The set of mutations referred to as F3 are: V68I, A204V, V208L, V255L, and H66S.

Elimination of A204V may be done if adding a V may be too large for the packed region where A204 resides. (Adding E causes opening of the apex.)

The set of mutations referred to as F4 are: V68I, S115V, V208L, V255L, and H66S.

Retention of N377L may be used for the minimal set. The above tested the effect of N377L elimination from the full set and whether N377L stabilizes.

The set of mutations referred to as F5 are: V68I, S115V, A204L, V208L, V255W, N377L, and H66S.

Addition of W69L to minimal set may be done as previous work suggests aromatic residues in position 69 are destabilizing and is tested here.

The set of mutations referred to as F6 are: V68I, S115V, A204L, V208L, V255L, and W69L.

Using W69V instead of W69L may be done to test whether side chain length alters potential stabilizing effect.

The set of mutations referred to as F7 are: V68I, S115V, A204L, V208L, V255L, and W69V.

Using W69A instead of W69L/V may be done to further test whether side chain length alters potential stabilizing effect.

The set of mutations referred to as F8 are: V68I, S115V, A204L, V255L, V208L, and W69A.

Reintroduction of M426W may be done to test a minimally reduced set and the effect of M's.

The set of mutations referred to as F9 are: V68I, S115V, A204L, V208L, V255W, N377L, M426W, and H66S.

Reintroduction of M434W may be done to test a minimally reduced set and the effect of M's.

The set of mutations referred to as F10 are: V68I, S115V, A204L, V208L, V255W, N377L, M434W, and H66S.

Introduction of additional H72P mutation may be done to test if P can favor loop turn stabilizing TRP69 Loop in the W bound state.

The set of mutations referred to as F11 are: V68I, S115V, A204V, V208L, V255L, H72P, and H66S.

Testing minimal set with H66K rather than S may be done if the charge is a better solution to polar switch.

The set of mutations referred to as F12 are: V68I, S115V, V208L, V255L, and H66K.

Elimination of H66S from F1 may be done though H66 may be important for loop configuration.

The set of mutations referred to as F13 are: V68I, S115V, A204L, V208L, V255W, N377L, M426W, and M434W.

The Minimal Set 2 may include the elimination of H66S and swapping of S115V for A204V; H66 could be important for loop and A204 my better stabilize that S115V.

The set of mutations referred to as F14 are: V68I, A204V, V208L, and V255L.

Minimal Set 3 may include adding N377L to test for further stabilization.

The set of mutations referred to as F15 are: V68I, A204L, V208L, V255W, and N377L.

### V3 Lock—Full Set

The set of mutations referred to as VT1 are: Y177F, T320L, D180A, Q422L, Y435F, Q203M, E381L, R298M, N302L, and N300L.

Elimination of R298M and E381L may be used to determine whether these two are stabilizing rather than destabilizing.

The set of mutations referred to as VT2 are: Y177F, T320L, D180A, Q422L, Y435F, Q203M, N302L, and N300L.

Elimination of E381L may be used to determine whether this residue is required to stabilize R298.

The set of mutations referred to as VT3 are: Y177F, T320L, D180A, Q422L, Y435F, Q203M, R298M, N302L, and N300L.

Elimination of R298M may be used to determine whether this reside stabilizes E381.

The set of mutations referred to as VT4 are: Y177F, T320L, D180A, Q422L, Y435F, Q203M, E381L, N302L, and N300L.

Retention of Y177F and Y435F may stabilize interior through H-bonding.

The set of mutations referred to as VT5 are: T320L, D180A, Q422L, Q203M, E381L, R298M, N302L, and N300L.

Retention of Y177F and Y435F while eliminating R298M and E381L mutations may be a minimal set avoiding possible problems from charged pair mutations.

The set of mutations referred to as VT6 are: T320L, D180A, Q422L, Q203M, N302L, N300L.

The Dennis Burton Set is a control for comparison.

The set of mutations referred to as VT7 are: R298A, N302F, R304V, A319Y, and T320M.

Elimination of D180A may be done as D180 appears to be destabilizing but may be stabilizing.

The set of mutations referred to as VT8 are: T320M, Q422M, Q203M, N302L, and N300L.

Addition of S174V may be done as S174 is on the periphery but may be stabilizing with a hydrophobe.

The set of mutations referred to as VT9 are: T320M, Q422M, Q203M, N302L, N300L, and S174V.

The Peter Kwong Set (DS-SOSIP.4mut) is an additional control set.

The set of mutations referred to as VT10 are: I201C, A443C, L154M, N300M, N302M, and T320L.

*In the above description, “elimination” means that F #N construct includes all F #N-1 mutations except the mutations identified as eliminated. In some embodiments, “retention” means the identified mutation is included.

Contemplated also are subsets of the mutations within a set. In a non-limiting embodiment, the mutations in Set F14 could be further parsed out to determined if there are fewer mutations or combinations of fewer mutations than in Set 14 which provide stabilization of the trimer.

Contemplated is any HIV envelope incorporating stabilizing mutations of the technology.

In order to examine the ability of these mutants to replicate the effects of BMS, we determined whether CD4 induces exposure of V3 and open state epitopes in the BG505 SOSIP constructs F14, Vt8 and a combined F14/Vt8. Specifically, we investigated CD4 induced 17B and 19B epitope exposure via surface plasmon resonance. The BG505 wild type SOSIP was used as a control with the antibodies immobilized on the chip. SOSIP samples were preincubated with excess CD4. Additionally, we investigated samples preincubated with CD4 and the BMS compound to determine whether BMS further stabilizes the trimer.

The results indicate that, while both the 17B and 19B epitopes are largely masked in all constructs, both epitopes are exposed in the WT protein in the presence of CD4. Both the F14 and F14/Vt8 SOSIPs eliminate triggering while BMS and the Vt8 compound reduce the response markedly with Vt8 increasing the effects of BMS.

The results indicate that the layer-1 and layer-2 locking mutations effectively prevent CD4 triggering. We next asked what structural changes lead to this reduction in triggering. We therefore determined a structure of a VRC01 bound BG505 F14 SOSIP trimer using cryo-electron microscopy.

The class averages in the upper left indicate the roughly eighty-six thousand particles display multiple orientations while the ab initio structure determination based upon these particles clearly identifies the trimeric SOSIP configuration and associated VRC01 Fab molecules. The final resolution of the structure determined via Fourier shell correlation was 4.4 angstroms yielding sufficient resolution in the region of interest for the F14 mutations at the core of the trimer to identify changes leading to the observed phenotype.

The difference between the initial model used in refining the SOSIP coordinates into the cryo-EM map and the final refined map are depicted here. The two regions here suggest a mechanism by which the F14 mutants prevent CD4 induced rearrangements. First, on the left, layer-1, in lime green, has rearranged, shifting tryptophan sixty-nine in its layer-2 pocket, in purple. Second, on the right, tryptophan five-hundred and seventy-one, in the buried gp41 three-helix bundle, has shifted away its pocket between layers 1 and 2. As layers 1 and 2 are known to play a role in allosteric control of the trimer closed to open state equilibrium, this structure suggests the F14 mutations have decoupled the layers from communicating CD4 binding to the gp120 to gp41 interface, which is a major point of contact in the closed state that must break.

The results from the BG505 F14 structure indicate layer-1 and layer-2 have been decoupled from the allosteric network, thus eliminating CD4 induced triggering of the Env. We next asked whether the F14 mutations would eliminate triggering in the chimeric CH848 DT and CH505 M5 SOSIP timers.

We therefore determined whether CD4 induces exposure of V3 and open state epitopes in a CH848 DT F14/Vt8 mutant as the F14/Vt8 mutant was most effective in the BG505 SOSIP. As multiple SOSIP designs are available for CH848, we determine whether the new designs improve over the previous stabilization methods.

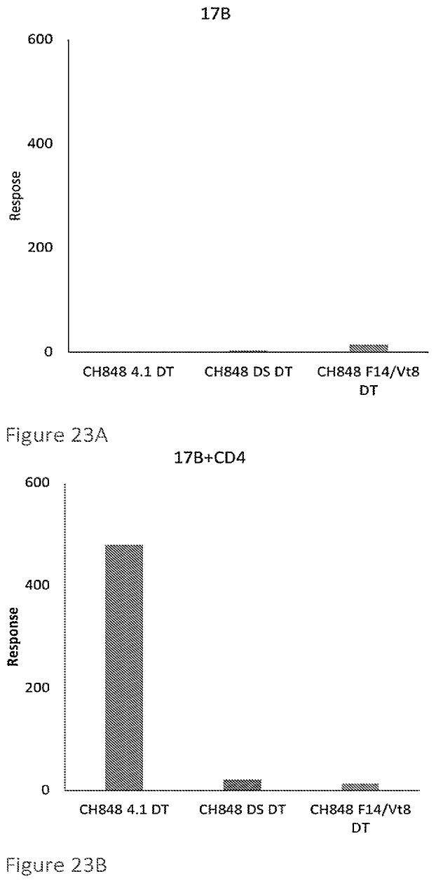

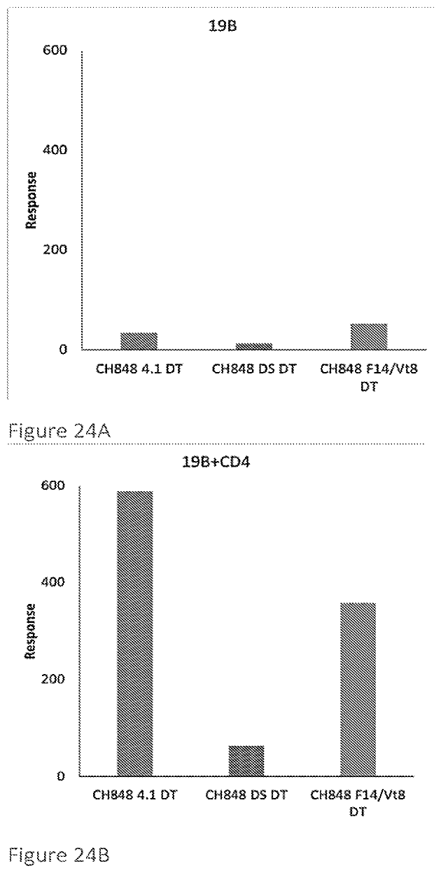

The results for the CH848 4.1 DT, CH848 DS DT, and the CH848 F14/Vt8 DT mutants indicate that while the 17B epitope is effectively triggered in the 4.1 stabilized mutant, both the DS and F14/Vt8 mutants are unresponsive. The 19B epitope is exposed in the presence of CD4 in the 4.1 and F14/Vt8 constructs with a minimal response from the DS construct. The BMS construct markedly reduces the response observed in the presence of CD4 (FIGS. 23 and 24).

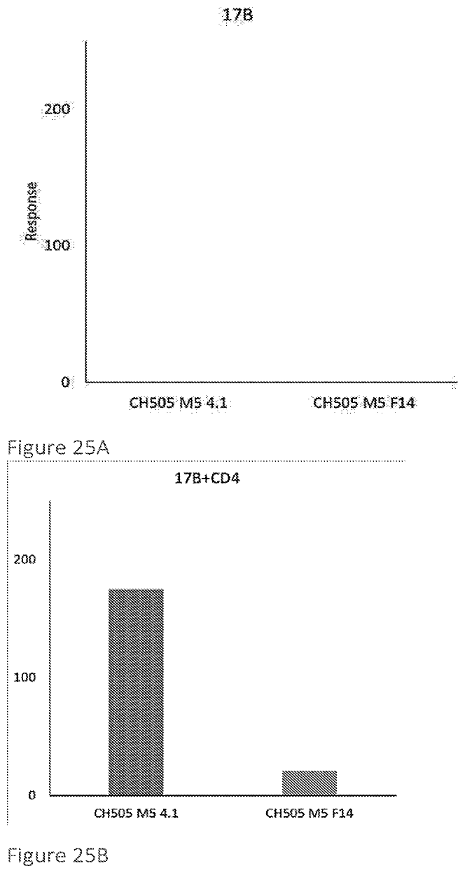

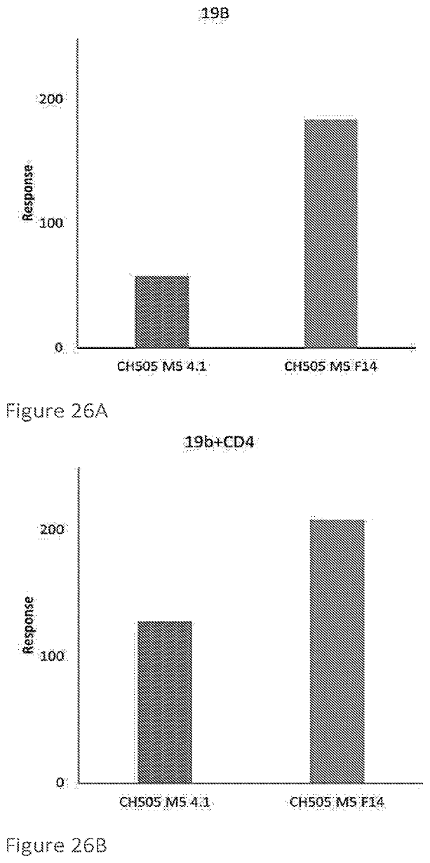

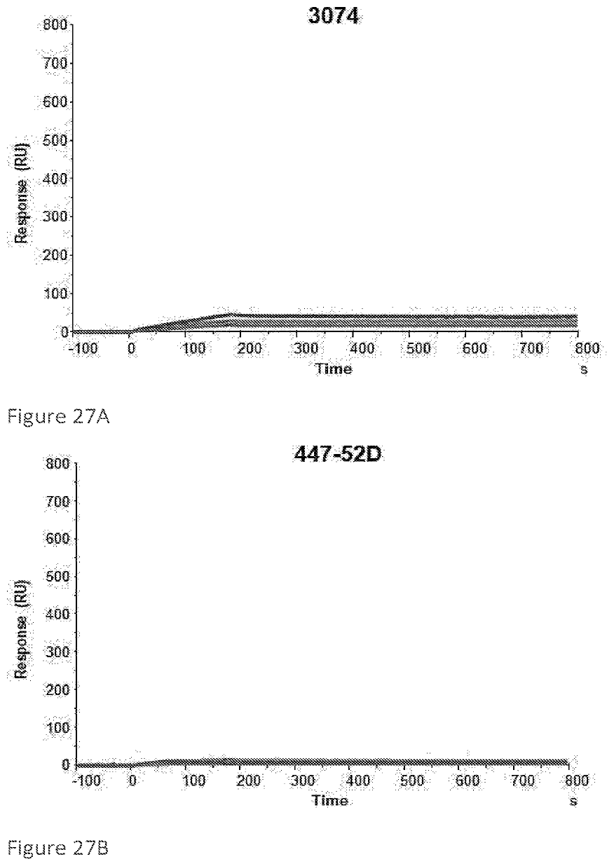

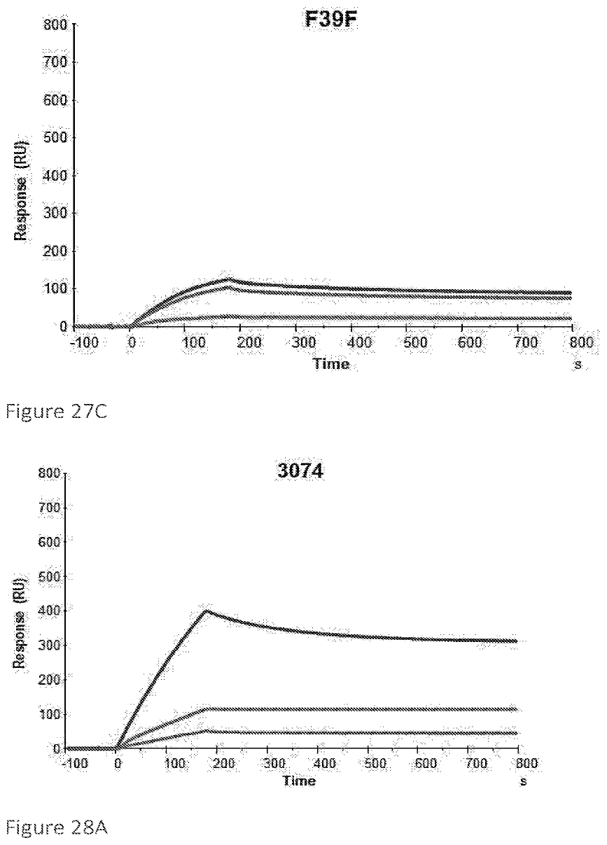

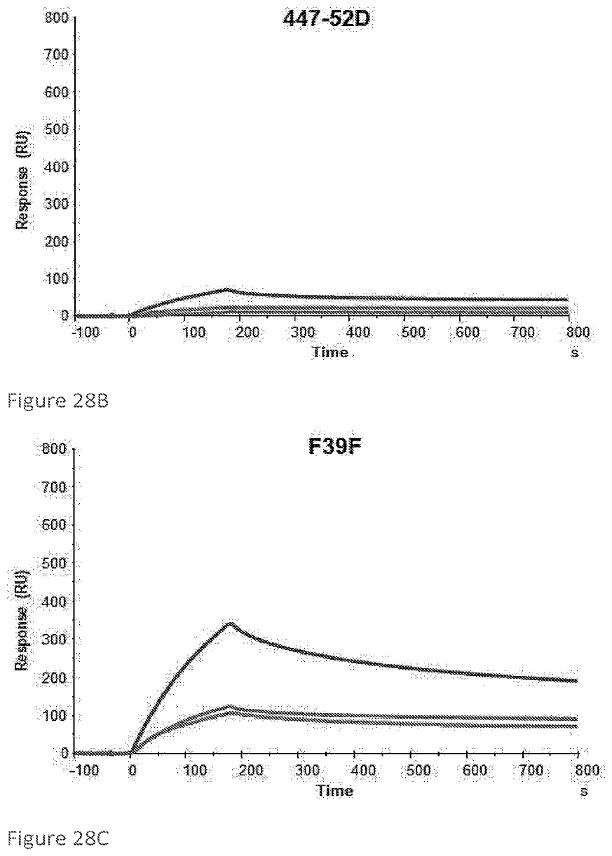

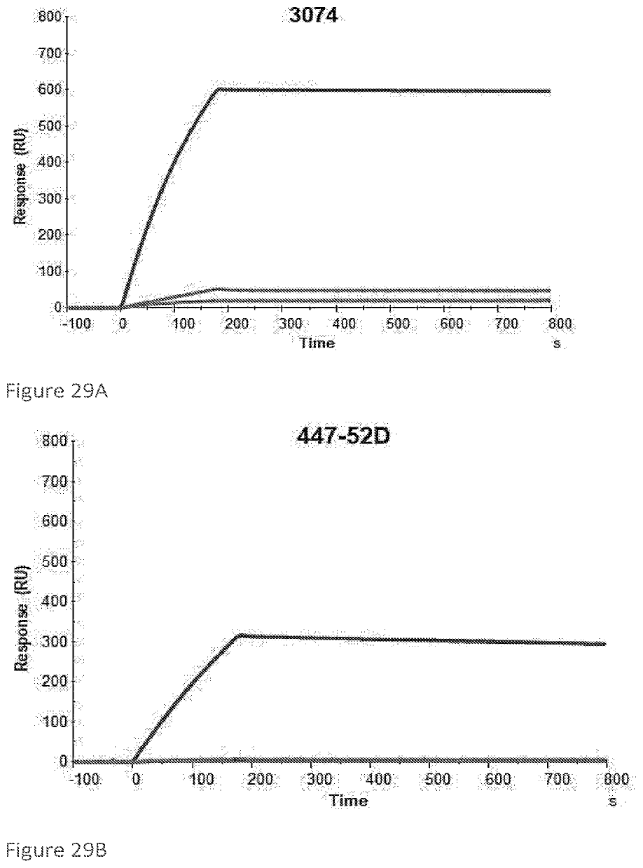

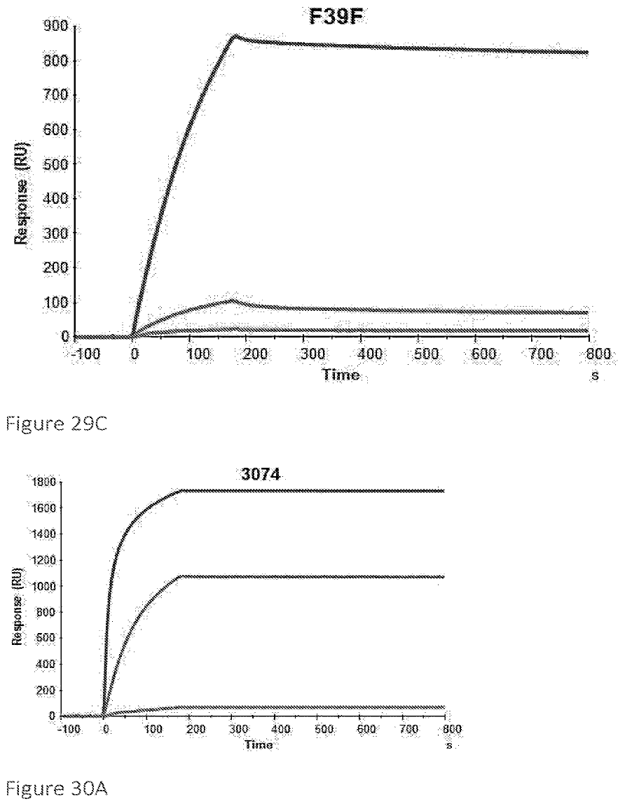

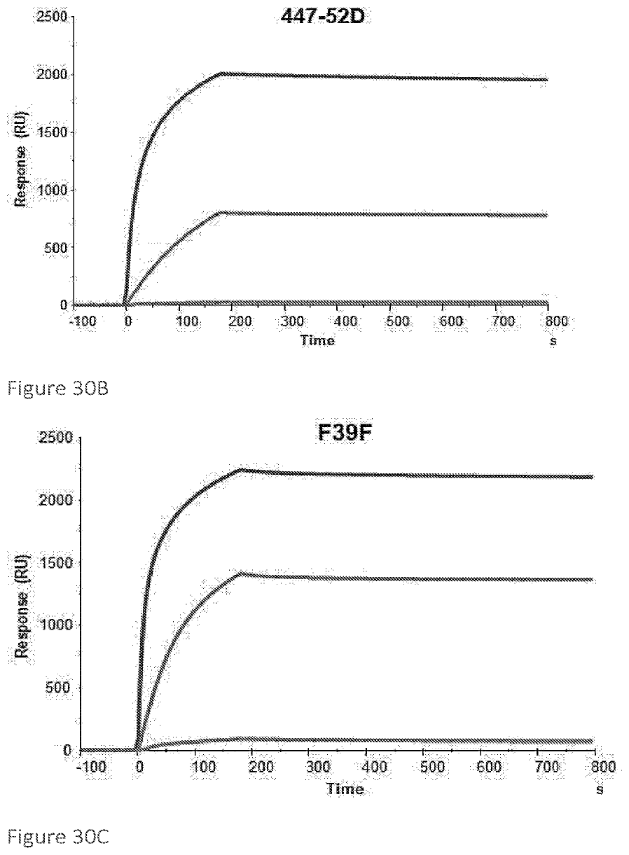

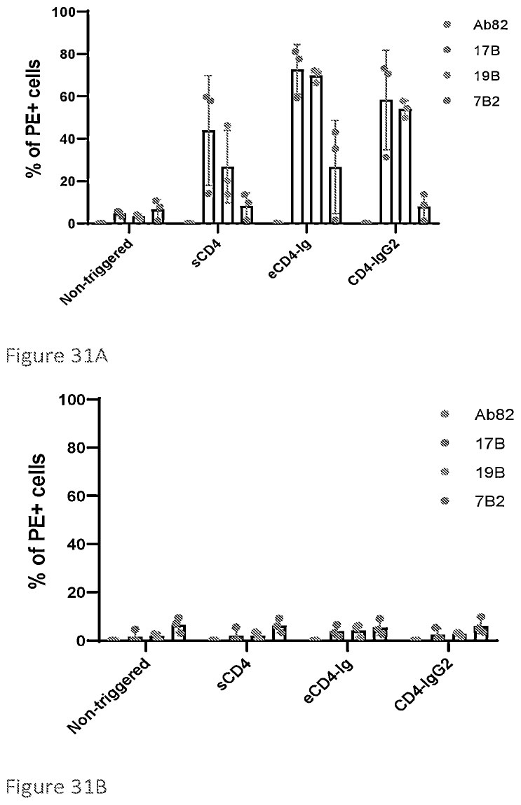

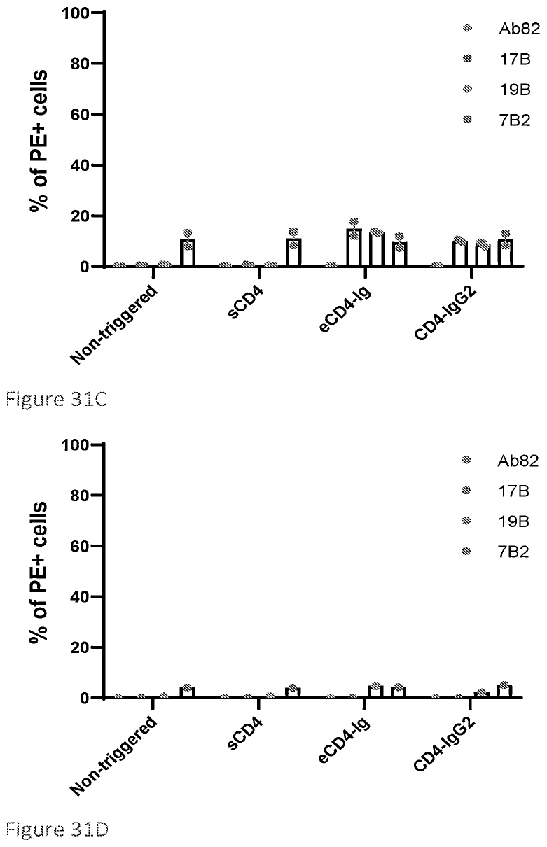

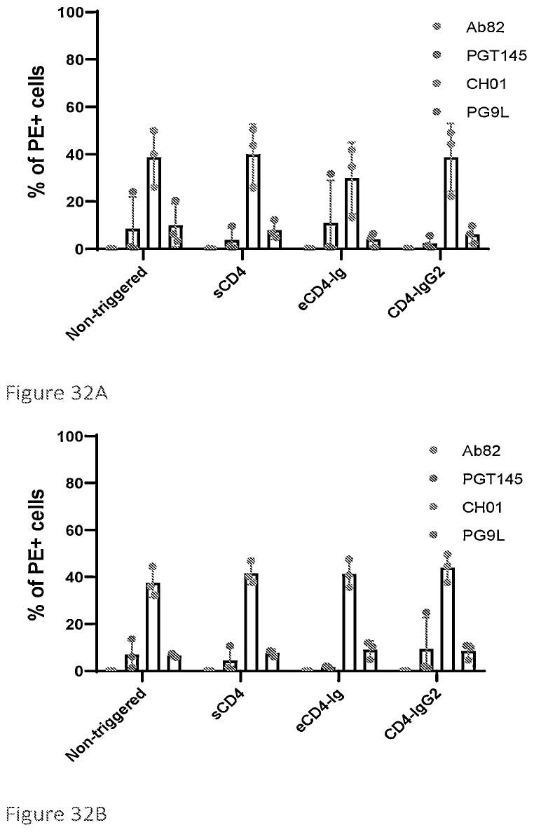

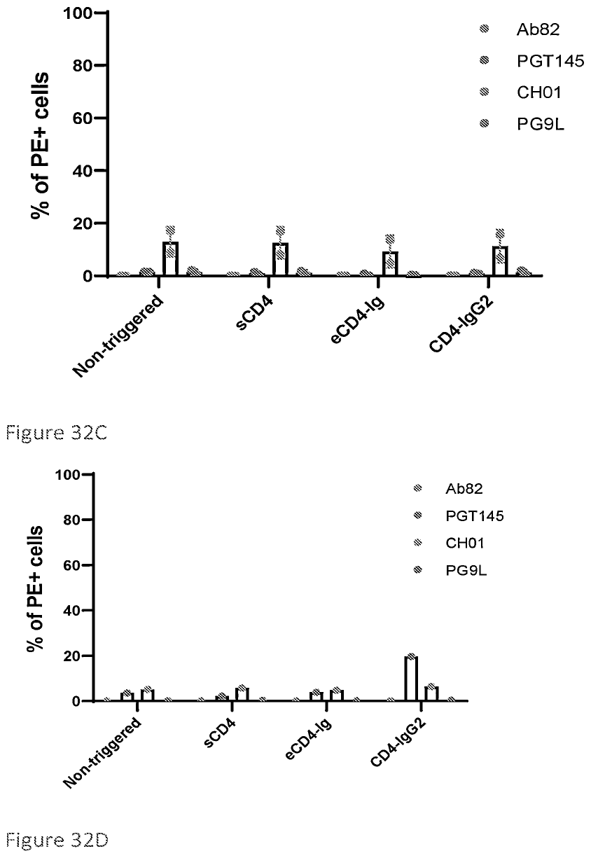

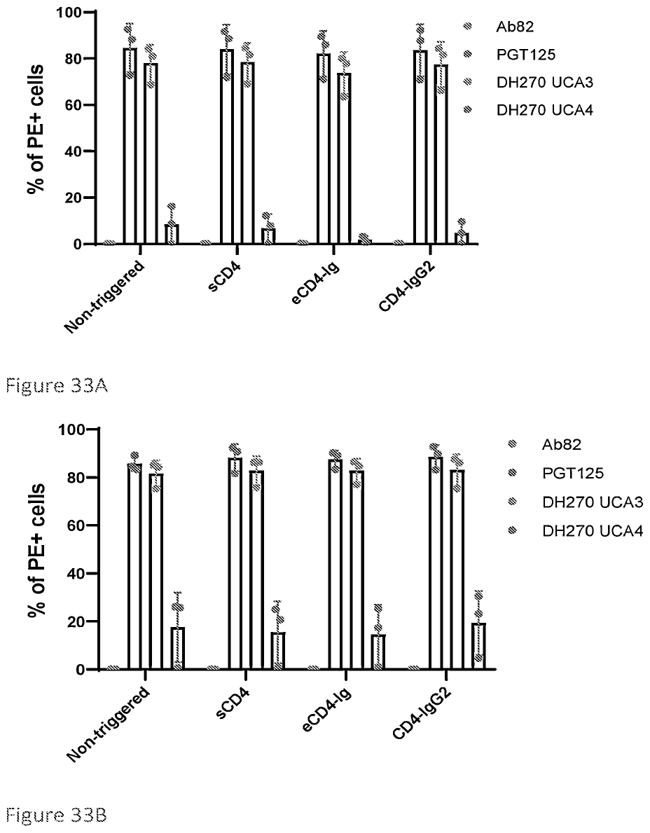

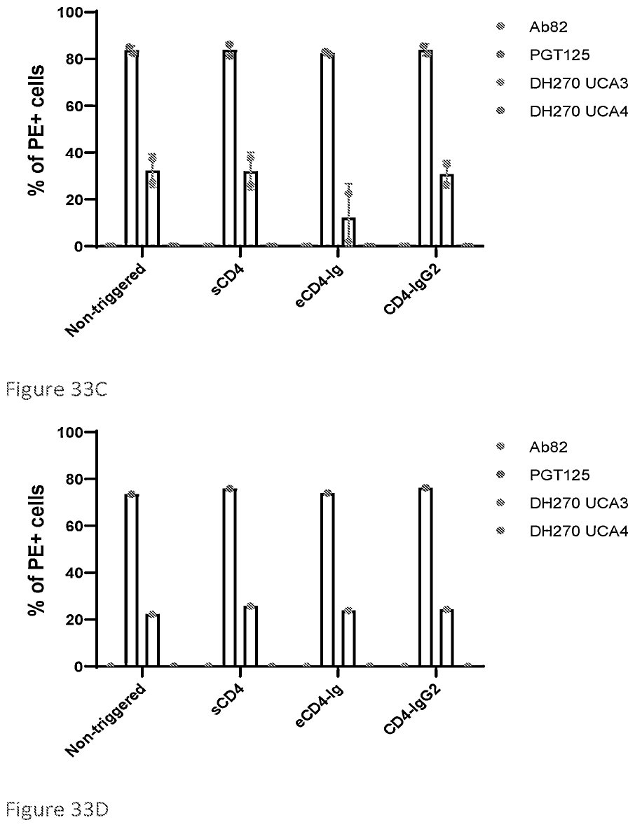

The results for the CH505 M5 4.1 and F14 mutants indicate that, the 4.1 mutant exposes the 17B epitope to a far greater extent than the F14 mutant. Though the 19B epitope is exposed in the 4.1 construct, triggering increases the extent of this exposure. Unusually, the F14 mutant exposes the 19B epitope to a greater extent than the 4.1 mutant and is largely unresponsive to the BMS compound. FIGS. 25 and 26.

The results indicate the F14 mutations effectively reduce exposure of the 17B epitope. However, this stabilization does not necessarily result in reduced 19B epitope exposure. In order to examine the potential effect of these mutations on other regions of the trimer, we next asked what effect these mutations have on the CD4 binding site.

SRP results indicate the F14 and Vt8 stabilizing mutations markedly reduce CD4 binding. It is therefore important to determine whether this effect extends to the CD4 binding site bnAbs. VRC01 binding was evaluated in the presence and absence of both CD4 and BMS.

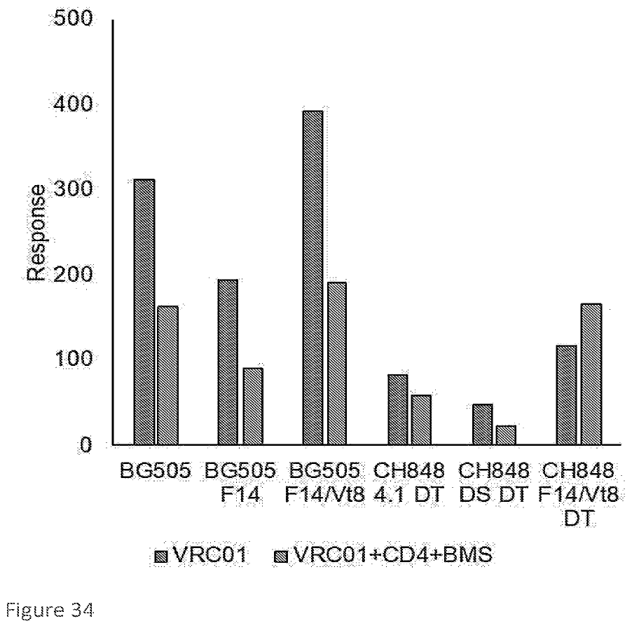

The results indicate that, while F14 displays a reduction in VRC01 binding in BG505, the F14/Vt8 combination increases this interaction. Interestingly, while addition of BMS in all BG505 constructs as well as the CH848 DS and 4.1 mutants reduces VRC01 interaction, the CH848 F14/Vt8 DT mutant displays an increased affinity for VRC01. FIG. 34.

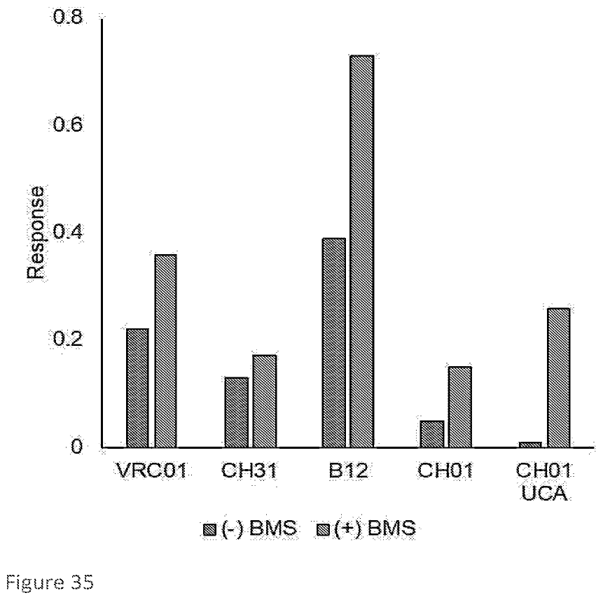

The results indicate that while F14 reduces VRC01 binding in BG505, the combined F14/Vt8 mutant increases VRC01 affinity in both BG505 and CH848 DT SOSIPs. Based upon the unusual increase in VRC01 binding of the CH848 F14/Vt8 DT SOSIP in the presence of BMS, we next asked whether this effect extends to other bnAbs. FIG. 35.

The results indicate that CH848 F14/Vt8 DT in the presence of BMS alone indeed increases VRC01 affinity. Additionally, increases are observed in CH31, B12 and CH01. Remarkably, though the CH01 UCA does not bind in the absence of BMS, the UCA interacts with the BMS bound trimer quite well. FIG. 35.

The results indicate the BMS compound is able to sensitize the CH848 F14/Vt8 DT SOSIP to interaction with multiple bnAbs and that the compound can induce interaction with the CH01 UCA.

In conclusion, the F14 and vt8 mutations limit CD4 triggering of the 17B and 19B epitopes in BG505 while the combined F14/Vt8 construct eliminates triggering. The F14/Vt8 mutations eliminate 17B exposure in CH848 DT and limit 19B exposure in the presence of CD4. Additionally, the CH848 F14/Vt8 construct displays a remarkable affinity increase in certain bnAbs in the presence of BMS. Finally, the F14/Vt8 mutations eliminate CD4 induced exposure of the 17B epitope though the 19B epitope remains exposed.

### EXAMPLE 2

Additional sequences comprising stabilizing modifications described herein are provided in WO2014/042669, WO/2017151801, WO/2017152146 and WO/2018161049.

These include sequences of envelopes designed to bind to UCAs of V1V2 and V3, such as but not limited to envelope 19CV3, which comprises the sequence of envelope CH0848.3.d1305.10.19 except for the V3 region which is derived from CH0848 D949.10.17. See FIGS. 52-53.

### EXAMPLE 3

Additional designs examining sub-combinations within a set may be designed to further reduce the number of amino acid changes. In a non-limiting example, set “F14” mutations may be reduced in designs as subsets comprising one, two or three combination of the four mutation currently included in set “F14”.

### EXAMPLE 4

Animal studies can be conducted to test the immunogenicity of the various immunogens.

Below is a sample protocol for a rabbit study using one of the immunogens of the technology.

Rabbit Study R47: BG505 gp140 SOSIP F14 x6 (IM)

Immunization #1-6
Immunogen: BG505gp140SOSIP.T332N.F14
Adjuvant: GLA-SE@0.1 mg/ml GLA, 4% SE (EM-107)
Rabbit #'s: M519, M520, M521, B514 (New Zealand White females)

Immunogen Dose: 100 μg per animal×5 animals (1 extra dose)=500 μg total
Adjuvant Dose: 25 μg GLA/2% oil per animal×5 animals=125 μg total GLA
Total Volume Needed: 500 μl per animal×5 doses=2.5 ml total

### Procedure

1. Calculate amount of protein needed based on concentration.
2. Add saline as needed to protein for an antigen volume of 1.25 ml
3. Add 1.25 ml antigen to 1.25 ml adjuvant for V_{final}=2.5 ml.
Inject 500λ per rabbit 250λ×2 sites IM (lumbar)
Prebleed
Immunization #1 (Week 0)
Post-Immune 1 bleed (Week 2)
Immunization #2 (Week 4)
Post-Immune 2 bleed (Week 6)
Immunization #3 (Week 8)
Post-Immune 3 bleed (Week 10)
Immunization #4 (Week 12)
Post-Immune 4 bleed (Week 14)
Immunization #5 (Week 16)
Post-Immune 5 bleed (Week 18)
Immunization #6 (Week 20)
Post-Immune 6 bleed (Week 22)

Samples from the various time points collected in this study are analyzed for binding, neutralization, etc.

### EXAMPLE 5

Based upon the similarity between the F14 and F14/Vt8 structures and those of the fusion peptide-antibody bound SOSIP trimers, we asked whether the F14 trimer would preferentially induce FP region antibodies in rabbits. That is, though immunization with SOSIP timer alone is unlikely to induce an FP targeting response⁷, was asked whether the stabilization of this region by the F14 mutations would induce phenotypically more homogeneous responses to the FP region at the gp120/gp41 interface relative to responses induced by a WT SOSIP trimer. Immunizations were carried out in four rabbits each using 100 ug per dose of either the BG505 WT SOSIP or the BG505 F14 SOSIP at time intervals of four weeks for a total of five immunizations using GLA-SE as an adjuvant. Pre-immunization sera were collected prior to the first immunization with post-immunization sera collected two weeks after each immunization.

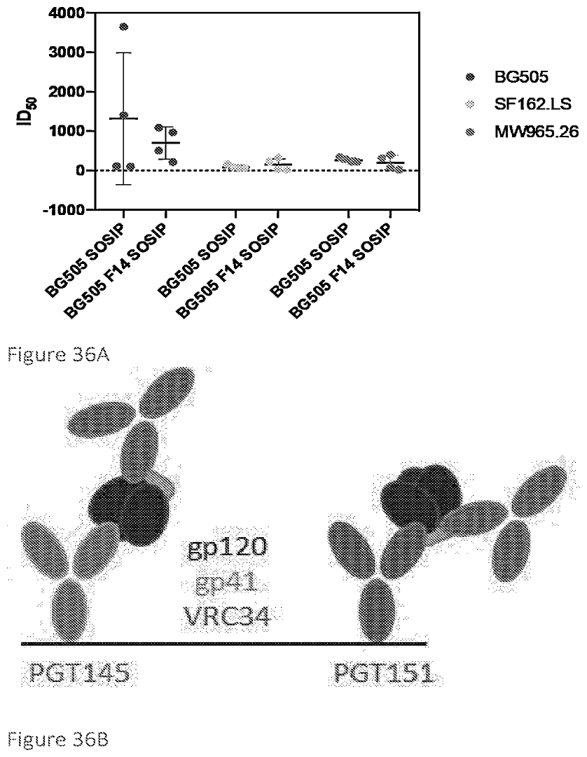

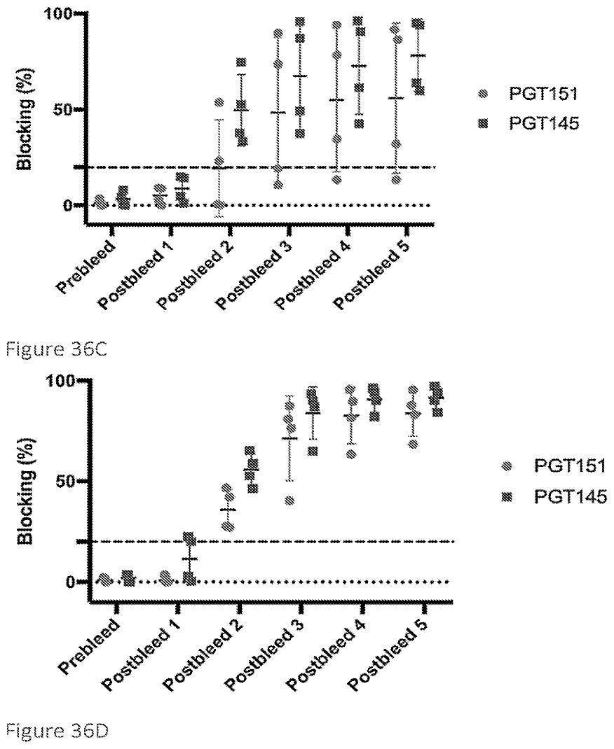

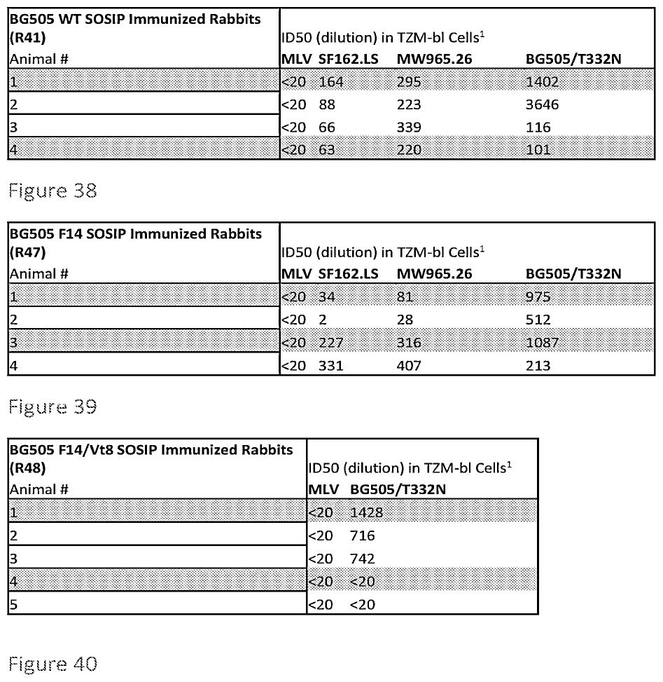

All four rabbits produced autologous tier-2 neutralizing responses in both the WT and F14 SOSIP immunized rabbits with geometric mean ID₅₀s of 495 and 583, respectively, after five immunizations (FIG. 36A, FIG. 38), while neutralization of the easy-to-neutralize tier-1 viruses SF162.LS and MW965.26 was minimal (FIG. 36A, FIG. 39). Though the F14 immunized group displayed a relatively tight titer distribution, the WT rabbits displayed widely disparate titers. Serum neutralization of autologous BG505 virus was observed in three out of five rabbits in a rabbit immunization study using BG505 F14/Vt8 as an immunogen (FIG. 40; data from post 4^{th} immunization sera). As not all rabbits produced autologous neutralizing antibodies, the BG505 F14/Vt8 SOSIP immunized rabbit sera was not further examined.

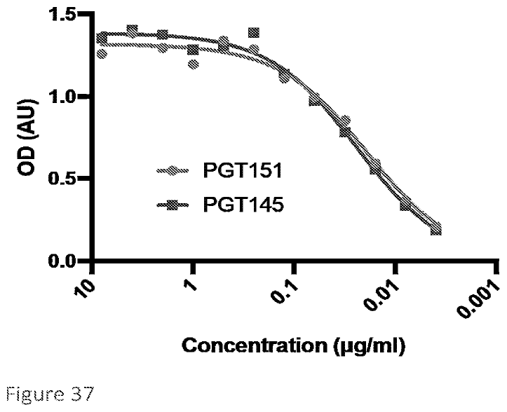

In order to compare the relative induction of FP region targeting immune responses in the BG505 WT and BG505 F14 SOSIP immunized rabbits, we next examined N123-VRC34.01 (VRC34) blocking of polyclonal post-immune sera binding to BG505 F14 SOSIP captured on ELISA plates by either PGT151 or PGT145 bnAbs. VRC34 is a broadly neutralizing FP antibody isolated from a HIV-1-infected individual that partially competes for the PGT151 Env gp41-gp120 epitope. We reasoned that phenotypically distinct binding responses would yield differential blocking of VRC34 when the SOSIP was captured using the apex targeting PGT145 vs. the gp120-gp41 interface targeting PGT151 as PGT151 is known to alter the gp120/gp41 interfacial conformation of all three protomers (FIG. 36B). Binding of VRC34 to BG505 F14 SOSIP captured by PGT151 and PGT145 revealed no effect of the capture method on the VRC34 interaction in our assay (FIG. 37). The WT immunized rabbits did not show blocking after the first immunization rising to ˜50% blocking after the second. Interestingly, while two rabbits in the WT immunization developed ˜95% blocking, two rabbits remained near ˜50% blocking (FIG. 36C). After the first immunization, two F14 immunized rabbits displayed weak blocking by VRC34 on PGT145 capture SOSIP rising to ˜50% blocking in all four rabbits after the second immunization and plateauing at ˜95% blocking after 3 immunizations in 3 rabbits and ˜83% blocking in one rabbit after 3 immunizations (FIG. 36D). Blocking of VRC34 on PGT151 captured SOSIP was not observed in either group after the first immunization, rising to ˜55% in 4 of 4 of the F14 immunized rabbits after the second immunization at which point only two WT immunized rabbits showed blocking (FIGS. 36C and D). The F14 immunized rabbits reached a PGT151 capture plateau at ˜95% blocking after the third immunization with the exception of one rabbit which plateaued at ˜75% after the fourth immunization (FIG. 36D). Two of the WT immunized rabbits displayed a similar plateau at ˜95% blocking. The two WT immunized rabbits which displayed weaker blocking using PGT145 capture displayed only weak blocking using PGT151 capture at a single time point each. Interestingly, the two low neutralization titer WT-immunized rabbits were those that displayed lower VRC34 blocking with sensitivity to PGT151 capture (FIG. 36D). Together, these data suggested that the BG505 F14 SOSIP presents a more homogenous, immunogenic configuration at this important site of Env vulnerability.

### EXAMPLE 6

### Disruption of the HIV-1 Envelope Allosteric Network Blocks CD4-Induced Rearrangements

The trimeric HIV-1 Envelope protein (Env) mediates viral-host cell fusion via a network of conformational transitions, with allosteric elements in each protomer orchestrating host receptor-induced exposure of the co-receptor binding site and fusion elements. To understand the molecular details of this allostery, we introduced Env mutations aimed to prevent CD4-induced rearrangements in the HIV-1 BG505 Env trimer. Binding analysis performed on the soluble ectodomain BG505 SOSIP Env trimers, cell-surface expressed BG505 full-length trimers and single-molecule Förster Resonance Energy Transfer (smFRET) performed on the full-length virion-bound Env confirmed that these mutations prevented CD4-induced transitions of the HIV-1 Env. Structural analysis by single-particle cryo-electron microscopy performed on the BG505 SOSIP mutant Env proteins revealed rearrangements in the gp120 topological layer contacts with gp41. Specifically, a conserved tryptophan at position 571 (W571) was displaced from its typical pocket at the interface of gp120 topological layers 1 and 2 by lysine 567, disrupting key gp120-gp41 contacts and rendering the Env insensitive to CD4 binding. Vector based analysis of closed Env SOSIP structures revealed the newly designed trimers exhibited a quaternary structure distinct from that typical of SOSIPs and residing near a cluster of Env trimers bound to vaccine-induced fusion peptide-directed antibodies (vFP Mabs). These results reveal the critical function of W571 as a conformational switch in Env allostery and receptor-mediated viral entry and provide insights on Env conformation that are relevant for vaccine design.

Host cell entry of HIV-1 is accomplished by the envelope glycoprotein (Env) spike. HIV-1 Env is a trimer of heterodimers comprised of gp120 and gp41 protomers, and exists in a metastable conformation capable of transitioning from a prefusion closed configuration to a fusion-competent open state upon triggering by CD4.^{1,2} The C-terminal gp41 domain contains a single transmembrane helix and the membrane fusion elements of the trimer.³⁻⁵ The gp120 segment binds the primary receptor CD4, triggering conformational changes leading to the binding of co-receptor CCR5/CXCR4 that causes global rearrangements in the trimer structure leading to viral and cell membrane fusion and gp120 shedding.⁶⁻⁸ While many studies have sought to understand the nature of the communication between the CD4 binding site, the coreceptor binding site and the fusogenic elements of the HIV-1 Env, a complete understanding of the allosteric mechanism and metastability in HIV-1 Env remains lacking.

The design of a soluble, stabilized ectodomain Env (SOSIP), containing an engineered gp120 to gp41 disulfide (SOS) and an HRI helix breaking I to P mutation, has revealed structural details regarding broadly neutralizing antibody (bnAb) epitopes and as an immunogen has induced autologous neutralizing antibodies.^{2,9} The Env open state presents highly immunogenic, conserved fusion elements that typically induce poorly neutralizing antibody responses with limited heterologous breadth.¹⁰ Indeed, design efforts to improve the Env SOSIP by further stabilizing the closed Env conformation have resulted in multiple prefusion stabilized trimer designs capable of inducing improved autologous, difficult-to-neutralize tier 2 virus antibody responses.¹¹⁻¹⁴ However, to date, no trimer design has successfully induced robust heterologous antibody responses.

Both the soluble and membrane-bound forms of the Env display an intrinsic ability to transition between multiple conformational states.^{2,15-17} In the pre-fusion, closed state, typical of SOSIP trimers, the gp120 domains surround a bundle of three gp41 helices, protecting conserved fusion elements of the trimer (FIG. 1A). Interprotomer gp120 contacts exist at the trimer apex and form a cap that further encloses the gp41 three-helix bundle. This cap is composed of three sequence variable loop regions termed V1/V2 and V3 (FIG. 1A).^{3,9,18} Global CD4-induced conformational changes result in dissociation of V1/V2 and V3 from the gp120 core toward a relatively disordered state as well as separation of gp120 from the gp41 three-helix bundle. This results in exposure of the three-helix bundle and fusion elements of the Env trimer.^{19,20} A layered architecture of the gp120 inner domain has been described²¹, with topological layers 1 and 2 contacting the gp41 subunit and shown, via mutagenesis coupled with cell-cell fusion and neutralization assays, to be important modulators of the CD4-bound conformation.²² Structures of the CD4-induced open state of the SOSIP Env, determined using cryo-electron microscopy (cryo-EM), showed that three gp120 tryptophan residues, W69, W112, and W427, form contacts from the CD4 binding site β20-β21 loop through to an intermediate helix, containing a portion of layer-2, to the distant gp120 layer-1 loop that is in contact with the gp41 three-helix bundle in the closed state (FIG. 7).²⁰ In the open state structures, rearrangement of the layer-1 loop, which contains W69, further suggested that layer-1 plasticity plays a role in conformational transitions.²⁰ Indeed, each loop has been implicated in the regulation of Env conformational transitions.^{22.23} Additional structural information from antibody-stabilized, closed-to-open state intermediates in SOSIP trimers suggests trimer opening occurs through ordered conformational transitions.²⁴

Despite these advances in our understanding of Env transitions, atomic level details of the allosteric mechanism by which CD4 induces transitions in the Env has remained elusive, thus limiting our ability to leverage such a mechanism for the development of vaccine immunogens. To investigate the allosteric mechanism of CD4-induced Env opening and to design SOSIP and membrane bound Env trimers that are resistant to CD4-induced structural changes, we aimed to interrupt the allosteric network responsible for CD4-induced triggering. The small molecule BMS-626529 is an attachment inhibitor that stabilizes the Env SOSIP in its prefusion closed state preventing CD4 binding and triggering.^{25,26} BMS-626529 neutralizes Env pseudoviuruses with nanomolar (nM) IC50, and has shown efficacy in blocking HIV-1 infection in vitro and in vivo.²⁵ Further, BMS-626529 is known to stabilize membrane Env gp160 in its closed, functional state²⁶, which is an important target in Env immunogen design efforts. Based upon the known allosteric control elements in the Env^{20,21,23} and atomic level details of the binding site of the BMS-626529 inhibitor²⁶, we designed mutations that disrupted the Env allosteric network and rendered it unresponsive to CD4-induced structural rearrangements. Structure determination via single particle cryo-EM revealed key details regarding how allosteric elements downstream from the gp120-CD4 contact control transitions of the trimer between the Env closed and open states. These results provide a means by which to control the Env conformational ensemble and reveal new details required for understanding the conformational plasticity of the HIV-1 Env.

### Results

### Design of an Allosterically Decoupled, Conformationally Stabilized Env Construct

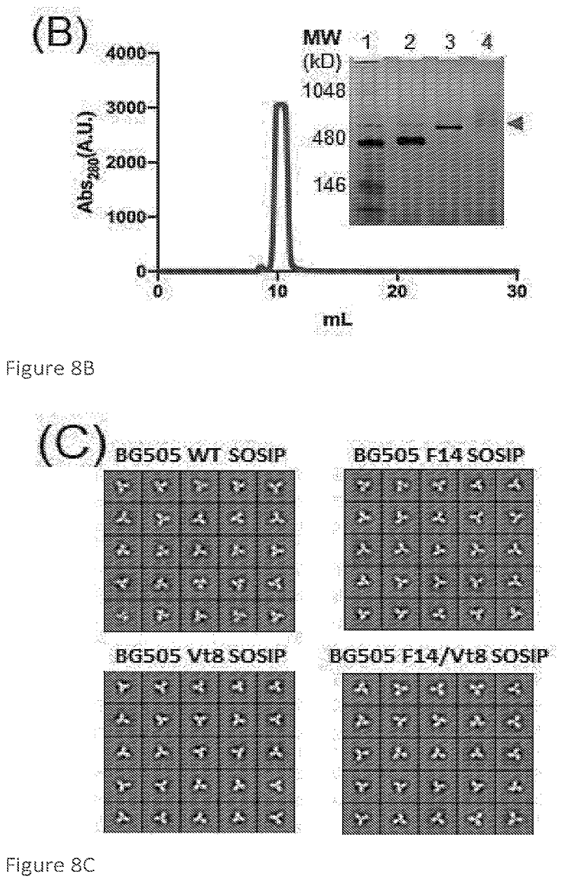

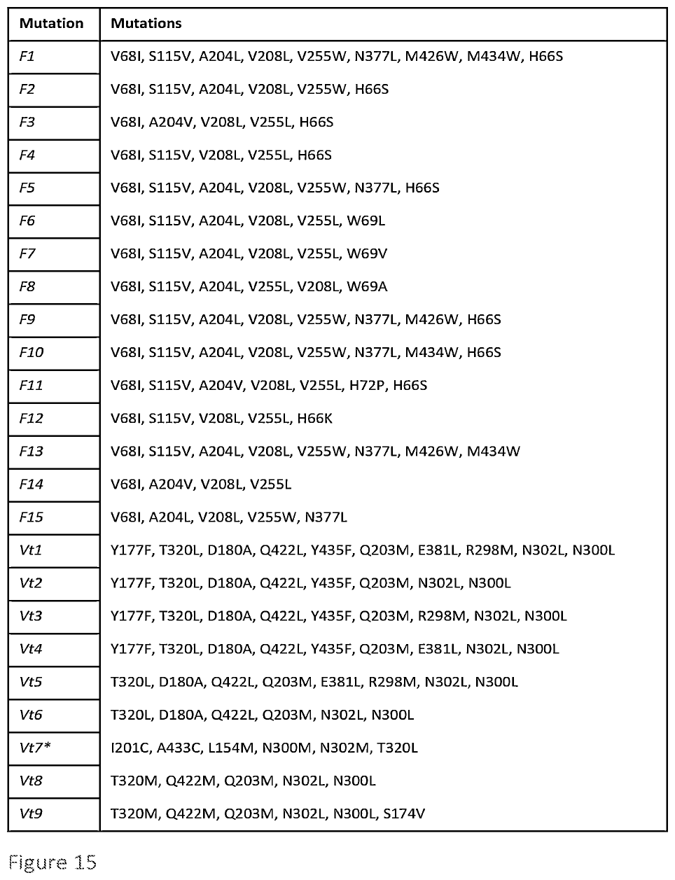

The small molecule HIV-1 entry inhibitor BMS-626529 has been shown to prevent sCD4-induced rearrangements in both soluble gp140 SOSIP trimers and native virion bound Env gp160.^{25,26} We reasoned that design of stabilized Env trimers based on an understanding of BMS-626529-mediated stabilization could lead to novel mutations that inhibit CD4 triggering for both the soluble SOSIP and membrane-bound Envs. A recent structure of a BG505 SOSIP in complex with BMS-626529 revealed that the compound resides in an induced pocket between the β20-β21 loop and the layer-2 α-1 helix, thus acting to separate the inner and outer domains (FIG. 1A).²⁶ The BMS-626529 compound appears to interrupt CD4 interaction by sequestering three key CD4 contact residues, N425, M426, and W427, thus impeding CD4 interaction and associated downstream rearrangements. Based upon the BMS-626529 contact region and neighboring residues in this structure, we selected clade A BG505 Env outer domain residues V255 and N377 and β20-β21 residues M426 and M434 for mutagenesis. (FIG. 1A). In combination with these residues, additional sites were selected in layer-1 residues V68, H66, W69, and H72, layer-2 residue S115, and V1/V2 region residues A204 and V208 in order to prevent transitions in these conformationally plastic regions (FIG. 1A). Together, this set of mutation sites, termed the F-series (FIG. 15), included residues spanning the entire allosteric network region of gp120 from the CD4 binding site to the closed state site of gp120 contact with gp41 HR1. While the F-series mutations were designed to block CD4-triggering, we reasoned that V3 exposure may occur even in the absence of full triggering of the Env²⁷ and examined residues in the V1/V2 to V3 contact region for mutagenesis to lock V3 in its prefusion, V1/V2-coupled state. We selected outer-domain residues E381 and Q422, V1/V2 region residues Q203, D180, and Y177, V3 residues R298, N300, N302 and T320, and residue Y435 for mutagenesis (FIG. 1A). This set of mutations, termed the Vt-series (FIG. 15), was introduced to prevent V3 exposure in a manner similar to previous stabilization strategies.^{13,28} Hydrophobic and/or space-filling mutations were made at each site for both the F-series and Vt-series sites. Beginning from a construct containing all sites in each series, smaller sets of mutations were prepared in order to examine the effect of particular mutations and to maximize the likelihood of identifying a suitable set of mutations for downstream processing (FIG. 15). In order to examine their effect on the SOSIP trimer, each BG505 SOSIP mutant and the unmutated BG505 SOSIP (BG505 SOSIP) was transfected in HEK Freestyle293 (293F) cells followed by cell culture supernatant screening via biolayer interferometry (BLI) (FIG. 8A). The antibodies used in this screening included PGT145, 17B, 19B, and VRC01 in order to assess trimer quaternary conformation, CD4i epitope exposure, V3 exposure, and gp120 folding, respectively.

Comparison of these results with the differences observed in the same assay for BG505 SOSIP in the presence and absence of BMS-626529 identified the ‘F’ series mutants F11, F14, and F15 as well as the ‘Vt’ series mutant Vt-8 as candidates for replicating the effects of the BMS-626529 compound. Each construct displayed a higher binding response to PGT145 and VRC01, no apparent 17B binding, and reduced 19B binding relative to the BG505 SOSIP (FIG. 8A; mutations from¹³ included for comparison). The Vt8 mutations consisted of the V1/V2 mutation Q203M, V3 mutations N300L, N302L, and T320M, and the outer-domain mutation Q422M (FIGS. 1B and D). The F14 mutations consisted of the layer-1 mutation V68I, layer-2 mutations A204V and V208L, and the outer-domain mutation V255L (FIGS. 1C and D). The F15 mutant included the F14 mutations in addition to a gp120 outer-domain mutation N377L with F11 including mutations S115V, H72P, and H66S in addition to the F15 mutations. We selected the F14 construct for further characterization as it possessed the fewest number of mutations relative to F11 and F15. Since the F14 mutations were primarily in the layer 1 and 2 regions and were predicted to block the transition after CD4-induced destabilization of V1/V2 and V3, we combined F14 and Vt8 (F14/Vt8) in order to minimize V3 exposure (FIG. 1D).

### Trimer Formation, Antigenicity, and Soluble CD4 (sCD4) Triggering of the Redesigned SOSIP Constructs

To evaluate the antigenicity of the designed Env mutants, we produced and purified the BG505 and mutant SOSIP Env variants by transient transfection in 293F cell culture followed by PGT145 affinity chromatography to select for well-folded trimers. The PGT145 purified material was further purified via size exclusion chromatography which resulted in a homogenous peak corresponding to the SOSIP trimer yielding a gp140 band when analyzed by non-reducing SDS-PAGE gel (FIG. 8B). We verified trimer formation for each construct by negative stain (NS) electron microscopy. The NS 2D-class averages of each construct confirmed that the mutants adopted a trimeric configuration similar to that of the BG505 SOSIP trimer (FIG. 8C).

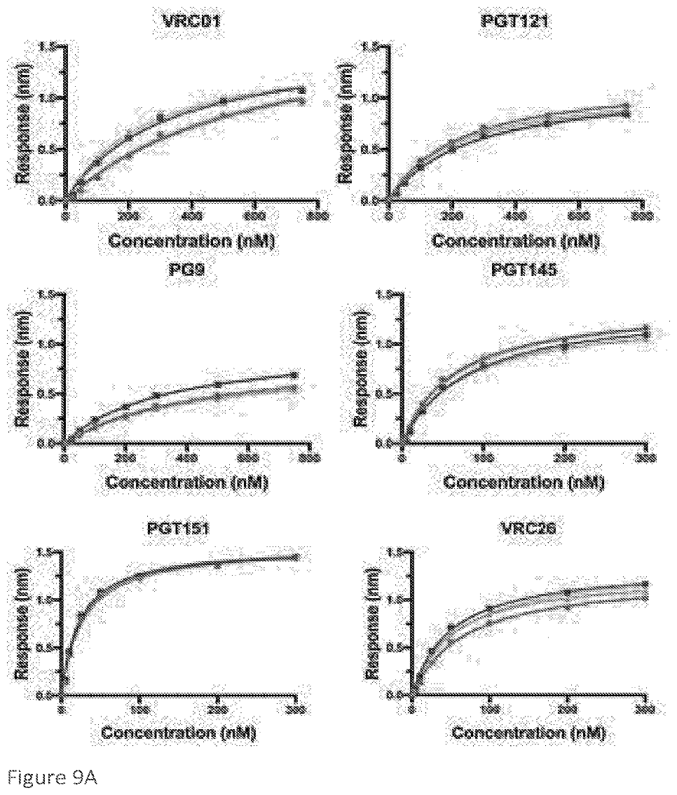

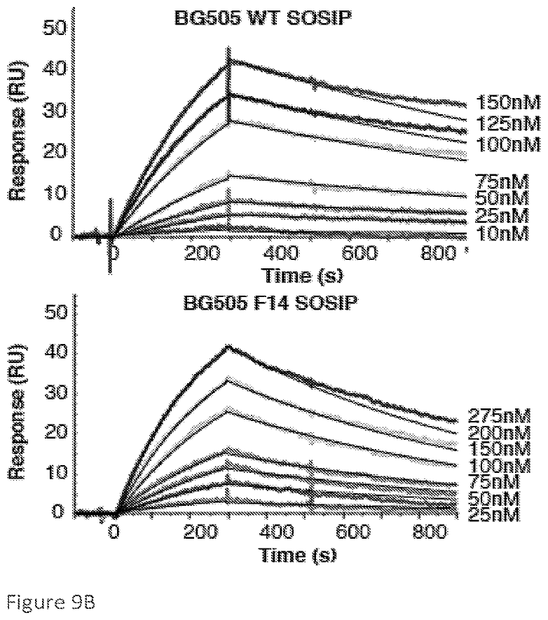

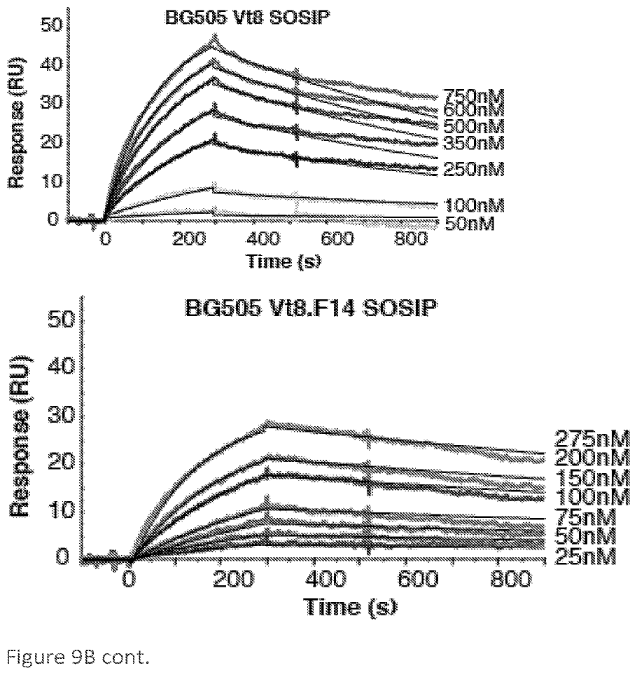

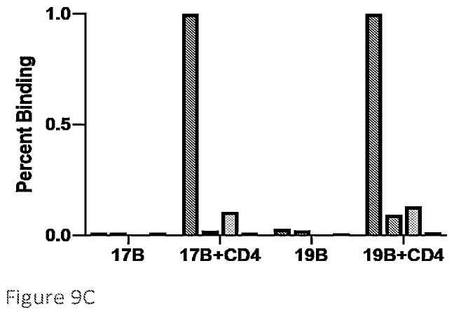

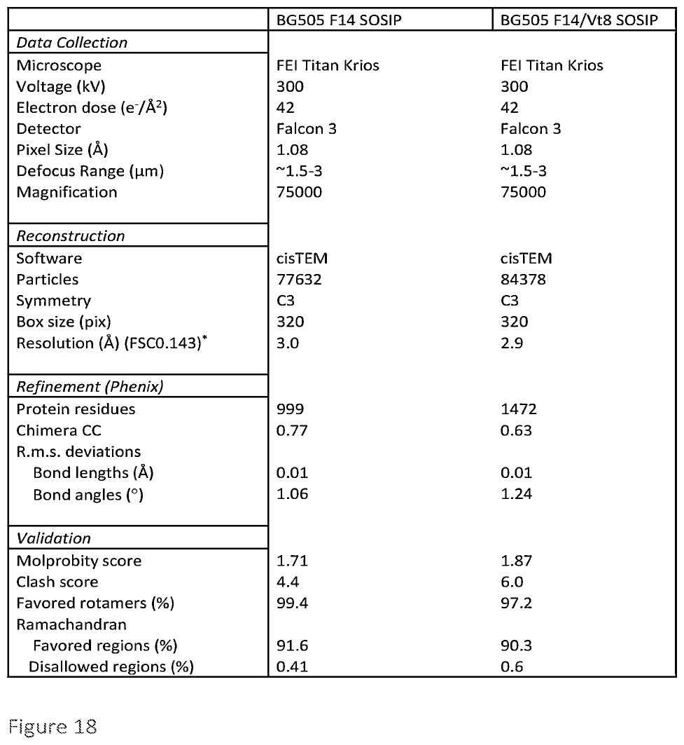

We next examined the antigenicity of key bnAb epitope specificities for the redesigned SOSIPs via BLI using VRC01, PGT121, PG9, PGT145, PGT151, and VRC26 bnAbs, having CD4 binding site, glycan-V3, glycan-V1/V2, trimer apex, gp120/gp41 interface, and V1/V2 epitope specificities, respectively (FIG. 1E). VRC01 binding indicated a ˜2-fold enhanced affinity for both Vt8 and F14/Vt8 relative to the BG505 SOSIP (FIG. 1E, FIG. 17, FIG. 9A). Fitting of the dose response curves for PGT121, PG9, PGT145, PGT151, and VRC26 indicated the mutations did not alter the affinity of the trimer for these important bnAb epitope specificities with nominal fold changes on the order of 1.0-1.3 (FIG. 1E, FIG. 17, FIG. 9A). These results indicated that the mutant designs presented a native, well-folded SOSIP trimer configuration and effectively presented multiple bnAb epitope specificities.

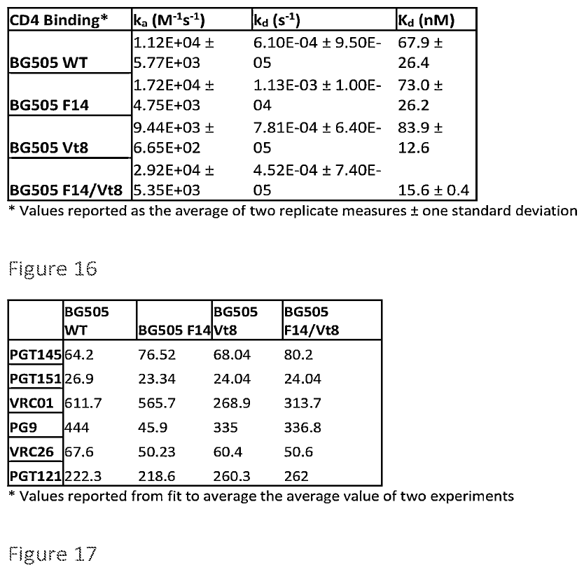

We next asked whether these mutations altered sCD4 binding. We determined the apparent affinity of each mutant construct and BG505 SOSIP for sCD4 via surface plasmon resonance (SPR). The affinities to CD4 determined for the F14 and Vt8 mutant SOSIPs matched that of BG505 SOSIP closely with K_{DS} of 73.0 nM±26.2 nM, 83.9 nM±12.6 nM, and 67.9 nM±26.4 nM, respectively (FIG. 1E, FIG. 16, FIG. 9B). The BG505 F14/Vt8 SOSIP construct displayed a ˜4-fold enhanced CD4 affinity compared to BG505 SOSIP with a K_{D} of 15.6 nM±0.4 nM primarily as a result of an enhanced association rate (FIG. 1E, FIG. 16, FIG. 9B). As each construct bound CD4, we next asked whether sCD4 triggering was inhibited by the F14, Vt8, and F14/Vt8 mutations relative to the BG505 SOSIP. The CD4i antibody 17B and V3-targeting antibody 19B were used to monitor triggering of the coreceptor binding site and V3exposed states, respectively. The results for the F14 construct indicated that sCD4 triggering of the open state is nearly eliminated based upon the lack of 17B response together with a ˜11-fold reduction in V3 response (FIG. 1E, FIG. 9C). The Vt8 mutations similarly reduced 19B epitope exposure by ˜7-fold with triggering of the 17B epitope reduced by ˜9-fold (FIG. 1E, FIG. 9C). Importantly, the combined F14/Vt8 construct eliminated CD4-induced exposure of both epitopes (FIG. 1E, FIG. 9C).

### Thermal Stability of F14 and F14/Vt8 SOSIPs

To assess the thermal stability of the F14, Vt8 and F14/Vt8 SOSIP trimers, and to compare them with BG505 SOSIP, we determined thermal denaturation maxima (Tₘₐₓ) using differential scanning calorimetry (DSC), which showed that that the F14 mutations did not alter trimer thermal stability with Tₘₐₓ values of 66.3° C.±0.02 and 66.3° C.±0.06 for the BG505 and F14 constructs, respectively (FIG. 2). The Vt8 and F14/Vt8 constructs displayed a 2.5° C.±0.02 and 1.8° C.±0.05 increase in Tₘₐₓ, respectively, indicating the Vt8 mutations slightly improved thermal stability of the SOSIP trimer (FIG. 2).

### Cryo-EM Structures of the BG505 F14 and F14/Vt8 SOSIPs

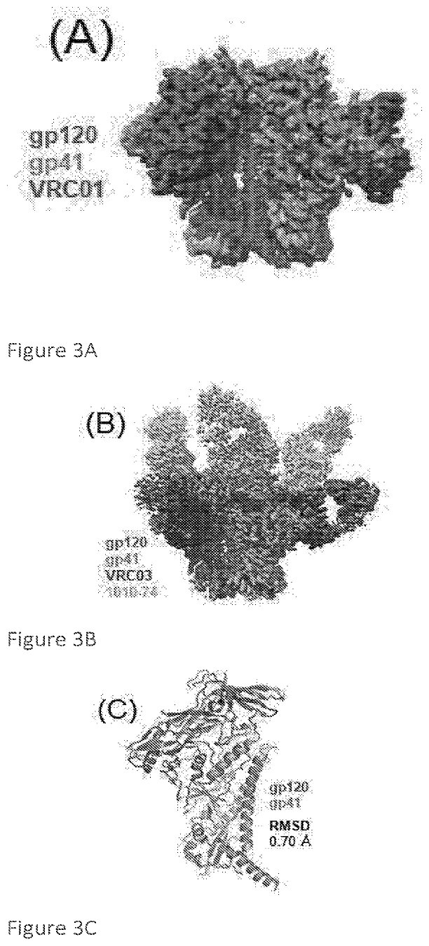

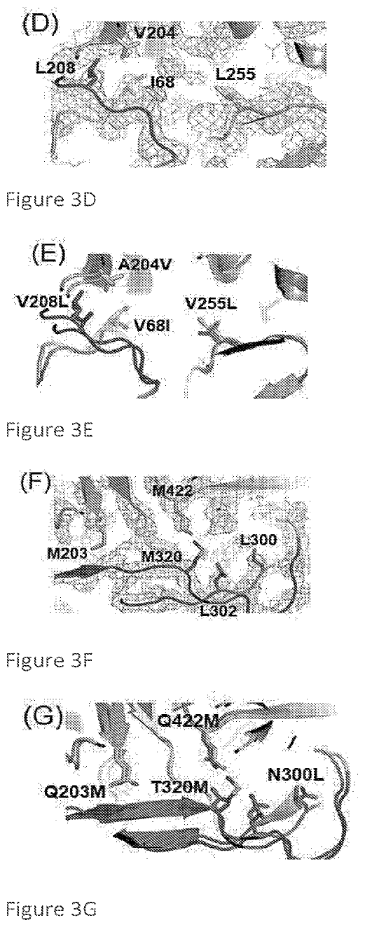

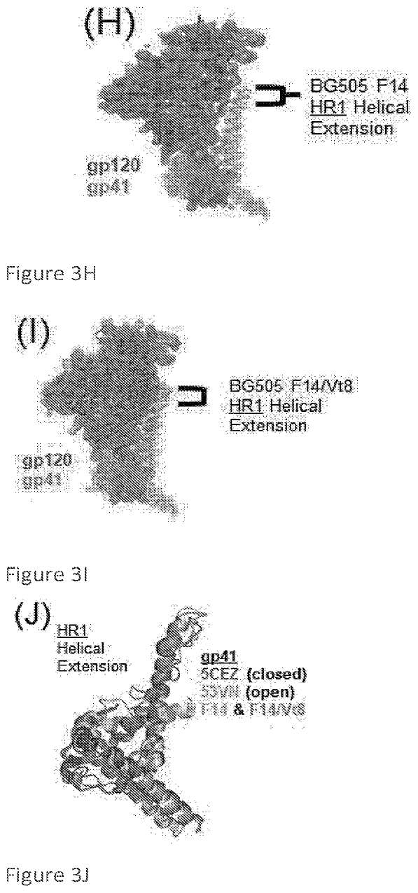

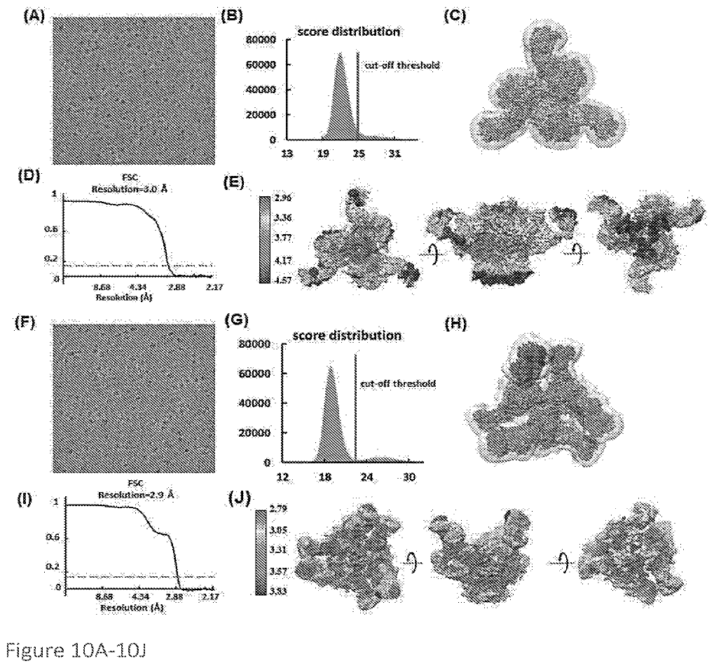

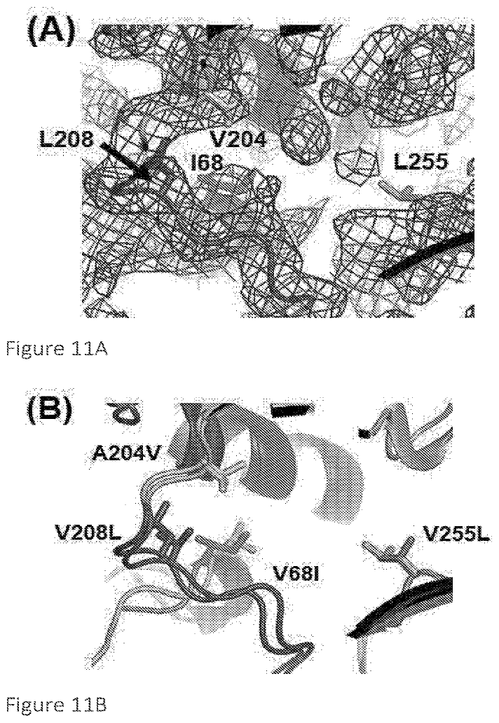

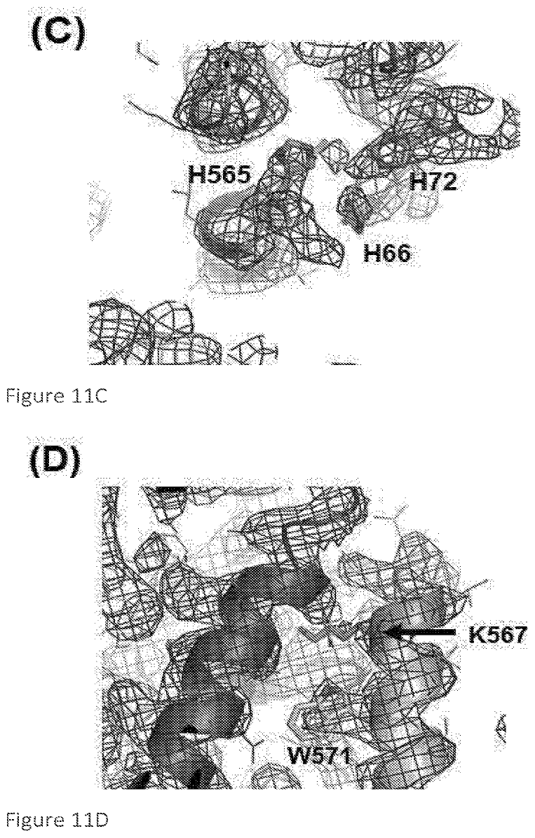

To understand the structural basis for the observed lack of CD4-induced conformational rearrangements, we determined structures of the F14 SOSIP trimer in complex with VRC01 (FIG. 3A) and the F14/Vt8 SOSIP trimer in complex with VRC03 and 10-1074 (FIG. 3B) via single particle cryo-electron microscopy (cryo-EM). Map reconstruction was initially carried out in cryoSPARC²⁹ followed by further refinement outside of cryoSPARC as described in the methods section (FIG. 10, FIG. 18). A total of 77,632 and 84,378 particles yielded final map resolutions of 3.0 Å and 2.9 Å, respectively (FIG. 10). Fitting of atomic coordinates into each map revealed similar overall structures for both BG505 F14 and F14/Vt8 SOSIPs with a root mean square deviation of 0.70 Å for gp140 alignment (FIG. 3C). The F14 mutations predominantly reside in V1/V2 near the apical region of layer-2 and in layer-1 near the gp120 contact with gp41 HR1 (FIG. 1A-C, 3D-G). Clear densities for the F14 and Vt8 mutations were observed in both structures revealing minimal change in their positions relative to their typical SOSIP positions (FIGS. 3D and F, FIG. 11).

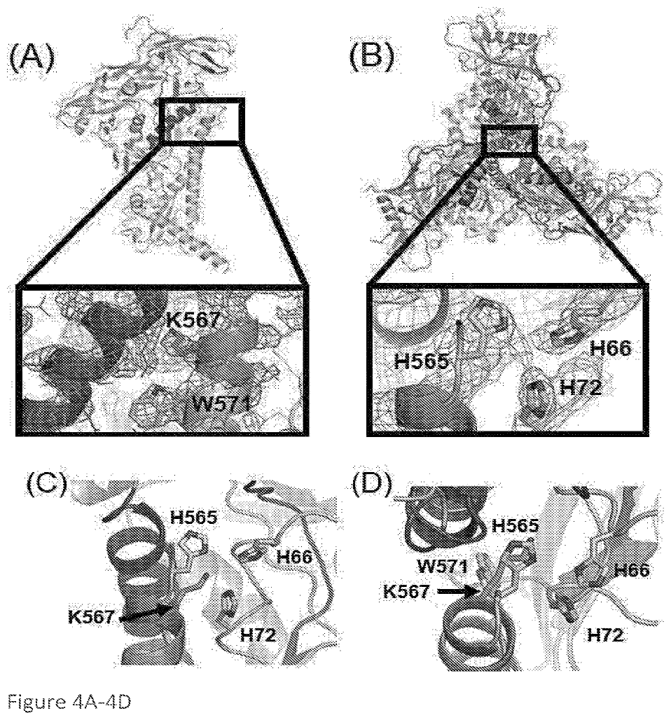

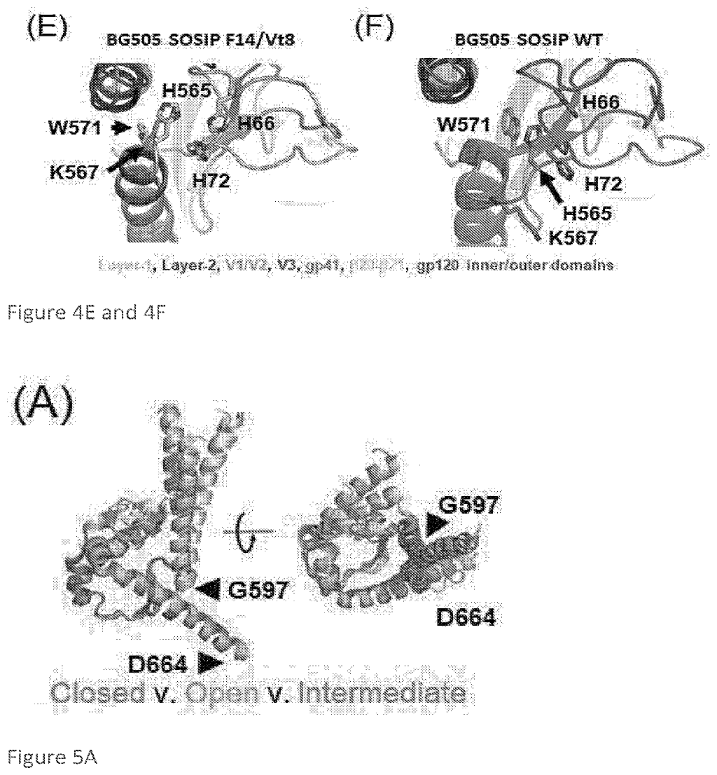

However, map densities in the C-terminal portion of HR-1 in both F14 and F14/Vt8 trimers displayed a helical extension of the buried three helix bundle toward the trimer apex, a feature that resembles the extension observed in open and partially open SOSIP structures (FIGS. 3H, I, and J).^{20,24} As a result of this gp41 restructuring, residue K567 displaced W571 from the layer-1/layer-2 pocket that was formed by residues F43, C54, W69, T71, A73, C74, D107, L111, and T217 (FIGS. 4A and D). In the F14/Vt8 structure, this restructuring was associated with a rearrangement in layer-1 that reoriented residues H66 and H72 resulting in the formation of a potentially water mediated histidine triad configuration with gp41 HR1 H565 (FIG. 4B, C, D). Interestingly, the F14 map displayed layer-1 loop densities suggestive of multistate behavior. Specifically, density corresponding to H66 and H72 in a configuration similar to that of the F14/Vt8 structure appeared alongside additional distinct densities, which may correspond to differing H72 sidechain configurations (FIG. 11). This suggested that the addition of the Vt8 mutations in the F14/Vt8 construct further stabilized the topological layer region facilitating the observed reduction in sCD4 triggering. Together, these results showed that structural changes in the region of gp120 contact with the C-terminal portion of gp41 HR1 caused by the F14 mutations effectively decoupled key allosteric control elements in the HIV-1 Env SOSIP trimer.

### Comparison of F14 and F14/Vt8 to Previously Determined Env Trimer Structures

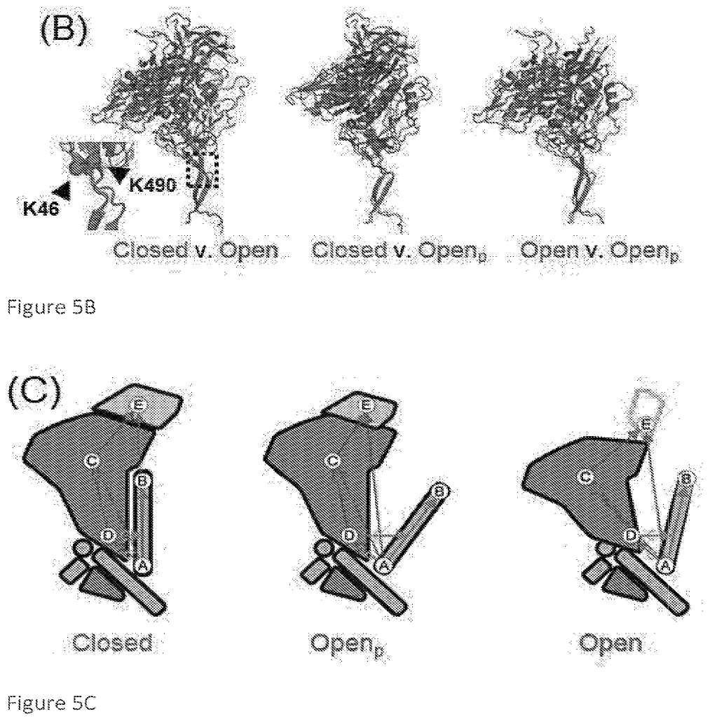

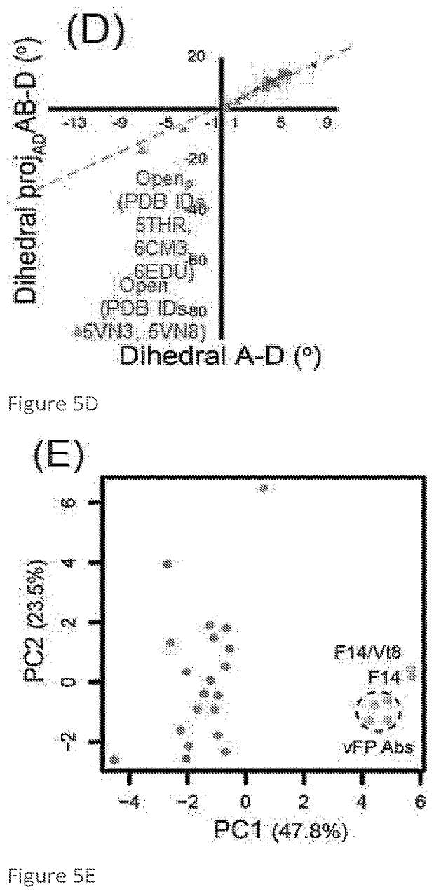

To understand the effect of the F14 and F14/Vt8 mutations on the overall structure of the SOSIP Env trimers, we examined regions of the structures distant from the F14 mutations. The individual domain coordinates of the mutant SOSIP trimer domains were found to be largely unperturbed indicating that the effects of the F14 and Vt8 mutations were localized. The gp120 domains within the Env Trimer are capable of rigid body movement relative to one another and to the gp41 three-helix bundle.³⁰ We therefore devised a set of reference positions in gp120 and gp41 capable of describing structural rearrangements associated with rigid body movement in gp120 and gp41 (FIG. 5). By comparing the closed³¹ and open state²⁰ SOSIP trimer structures we identified two key points in the trimer about which the distance and angular disposition of the relevant domains could be described. Specifically, alignment of closed and open state (PDB IDs 5CEZ and 5VN3, respectively) gp41 residues G597-D664 that occur at the C-terminal end of the three-helix bundle helix demonstrated that the conformational transition from closed to open state yields a similar structure in this region with an RMSD of 0.721 Å (FIG. 5A). This identified W596 as a hinge point about which the gp41 C-terminus rotates relative to the three-helix bundle helix. Comparing the position of the gp120 domains from this alignment revealed an additional trimer hinge-point between the gp120 N-terminal K46 and C-terminal K490 about which gp120 rotates (FIG. 5B).

We therefore devised a set of vectors connecting key points of the various trimer elements, including the W596 (A) and W571 (B) c-αs, a gp120 c-α centroid (C), a K46-K490 c-α centroid (D), and a V1/V2 c-α centroid (E), to enable comparison of the relative dispositions of the trimers in the closed, open and partially open (Openₚ) states (FIG. 5C). Analysis of the dihedrals formed by the W571 to W596, W596 to K46-K490 c-α centroid, and the K46-K490 c-α centroid to gp120 c-α centroid vectors and the W571 to W596/K46-K490 projection, W596 to K46-K490 c-α centroid, and the K46-K490 c-α centroid to gp120 c-α centroid vectors resulted in a linear relationship between the dihedral values for the closed state SOSIP Envs clustered at positive values (R²=0.98) (FIG. 5D). Partially open states lie on the same line but have negative values for both dihedrals while two open state structures occupy negative positions far from the linear fit to closed state structures (FIG. 5D).

The ability to discern closed, intermediate open, and fully open state structures confirmed that both dihedrals effectively reported on the relative disposition of gp120 to the gp41 three-helix bundle. We therefore examined whether the distance and angle terms between vectors connecting the key points, in combination with the dihedral terms, further characterized structural similarity between various antibody bound trimers. The spread in the angle and distance distributions limited direct analysis. Therefore, we employed the principal components analysis method to reduce the dimensionality of the dataset to enable clustering of similar SOSIP Env structures. The results indicated that the F14 and F14/Vt8 structures indeed differ from the majority of the available SOSIP structures, residing instead near a small cluster of fusion peptide-directed antibody-bound SOSIP structures (FIG. 5E).³² This finding indicated that the changes observed in the layer1/2 contact region with gp41 indeed altered the overall orientation of gp120 relative to gp41, and showed that these changes were structurally similar to those induced by fusion peptide-directed antibodies. We next examined the structure of the vFP antibodies in the region of the F14 conformational changes. All four vFP-bound, stabilized BG505 SOSIP trimers displayed density consistent with the helical extension and W571 rearrangement suggesting the rearrangement in the F14 and F14/Vt8 structures represents a functionally accessible state of the Env and that vFP antibodies may neutralize via inhibition of HIV-1 Env triggering (FIG. 5F, FIG. 12). Together, these results demonstrated the F14 and F14/Vt8 mutations induced global rearrangements in the SOSIP that may represent an accessible state to the native Env.

### Antigenicity, CD4 Triggering, and Conformational Distribution of the Redesigned gp160 Trimers

To determine whether the effects of the F14/Vt8 mutations observed in the soluble SOSIP trimers translated to native, membrane-bound gp160 trimers, we used two measures: 1) we assessed the antigenicity and CD4-triggering of cell surface Env gp160s using 293F cells displaying full length trimers on their surface and 2) we performed smFRET experiments on BG505 Env on the virion surface.

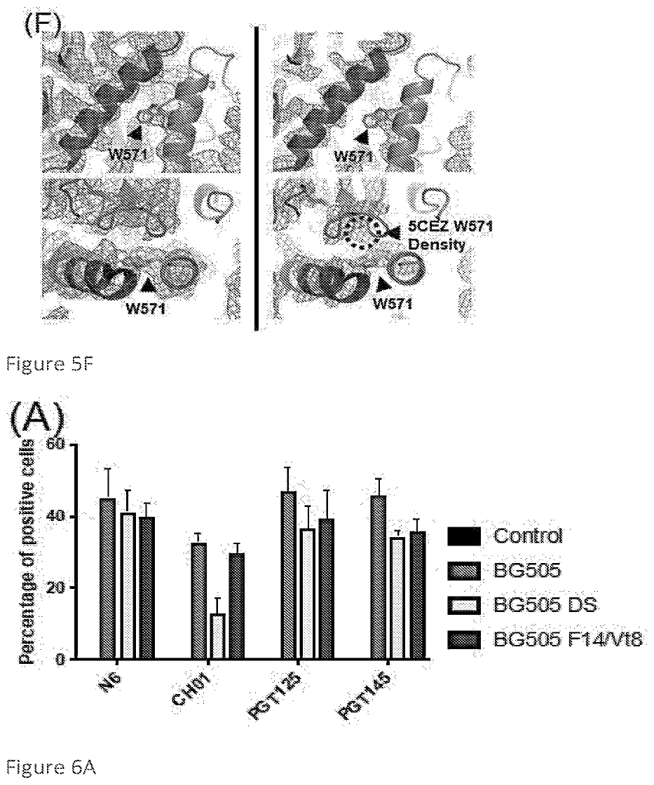

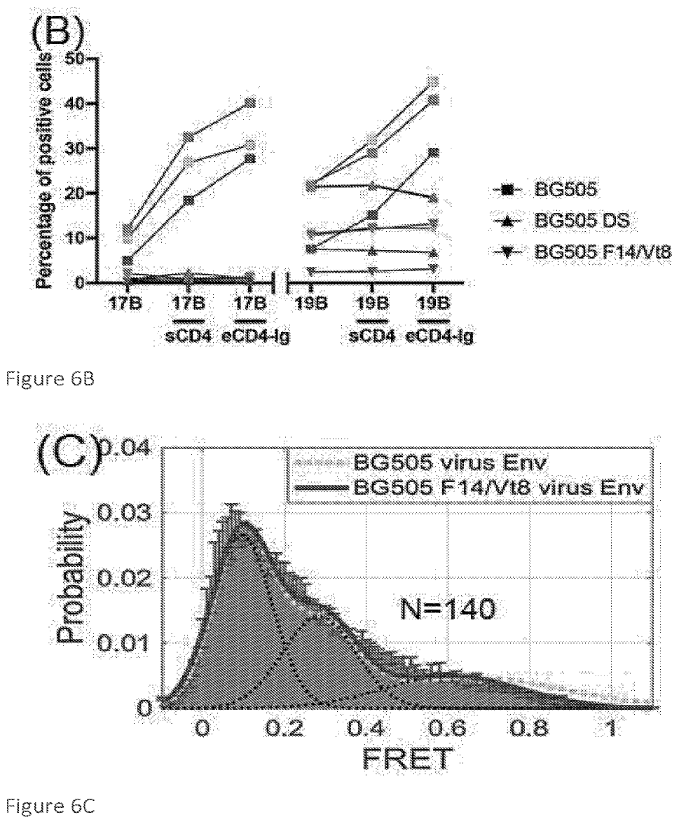

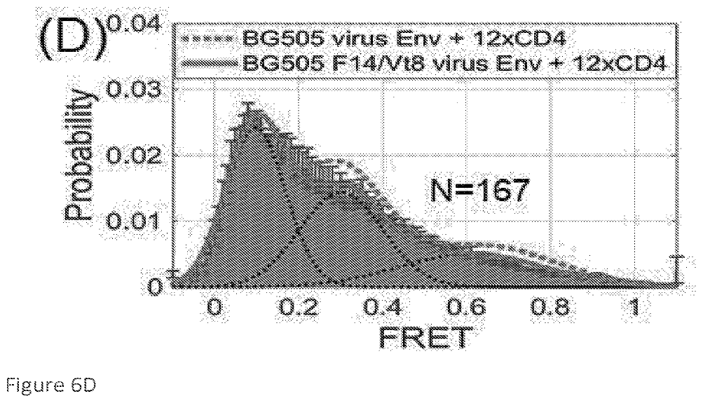

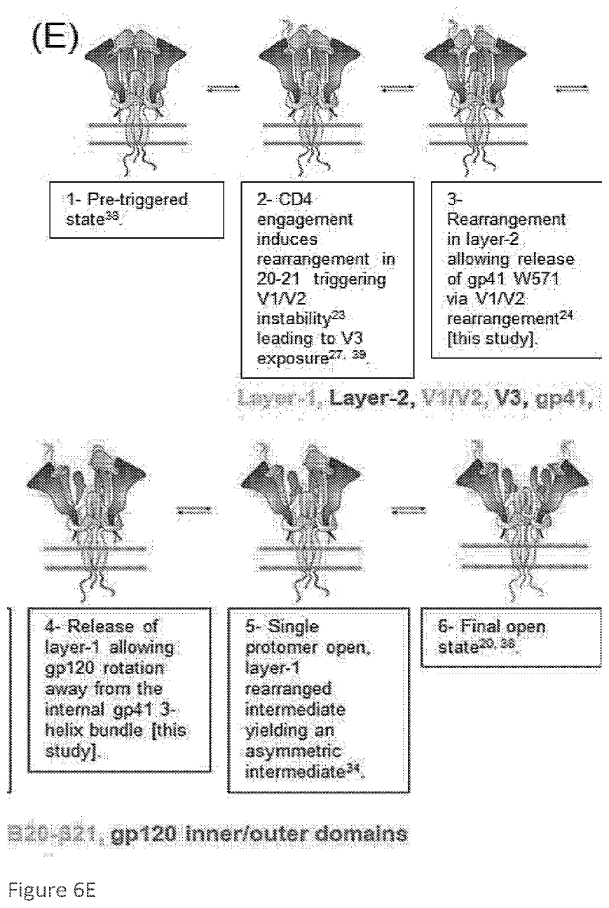

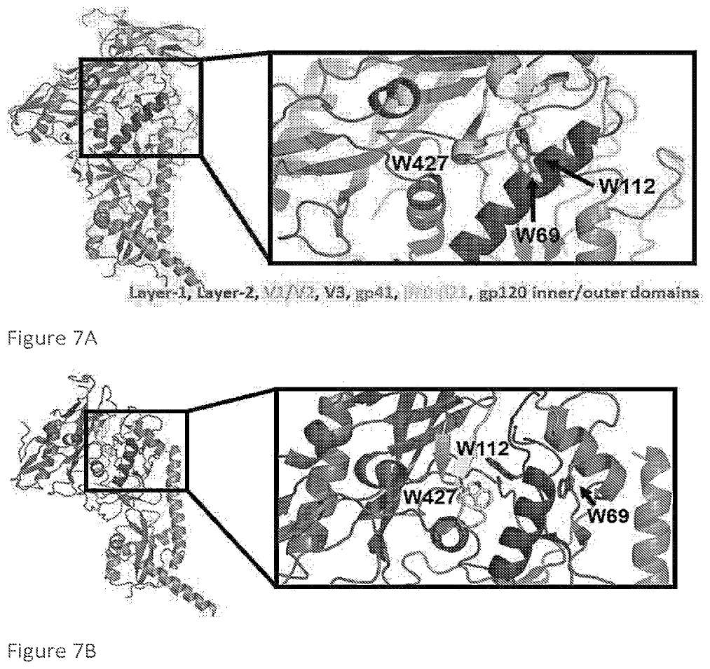

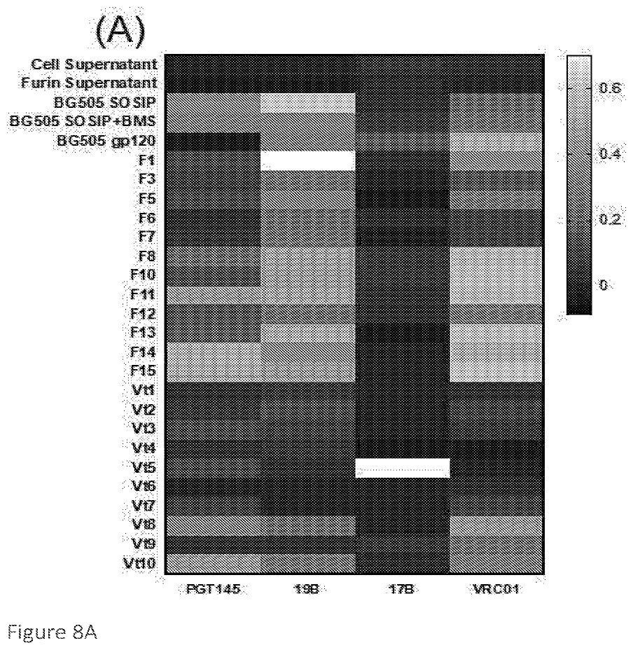

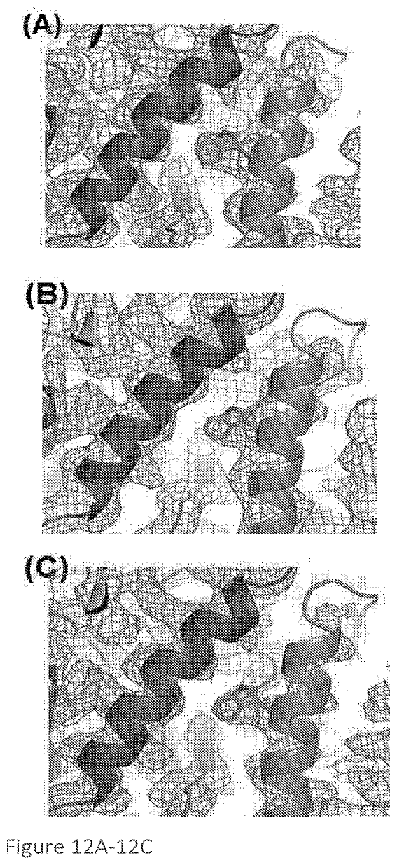

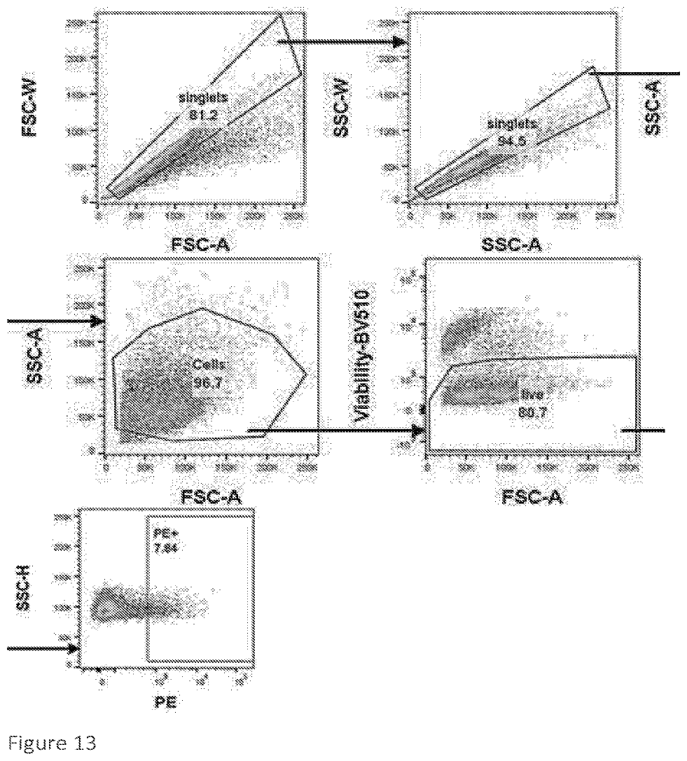

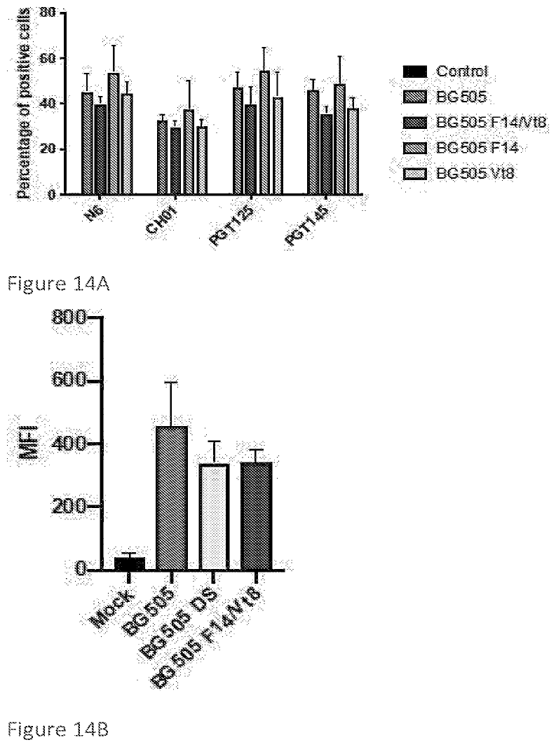

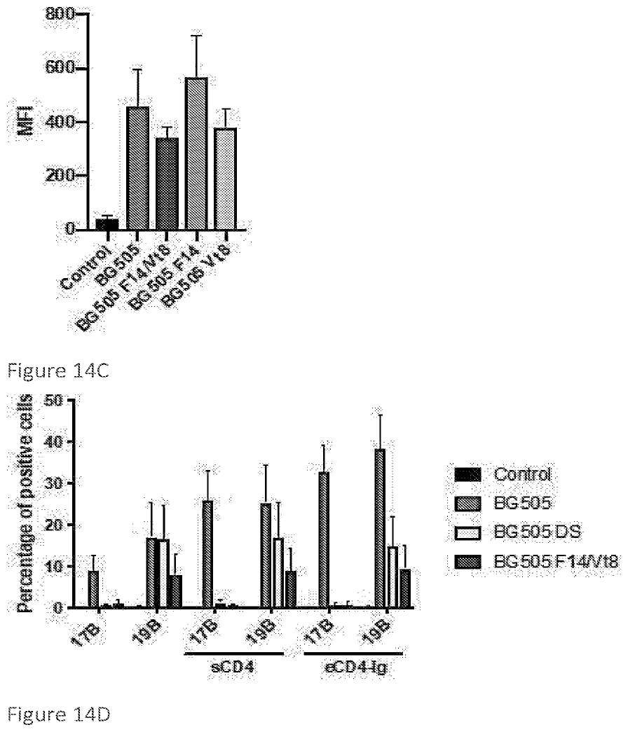

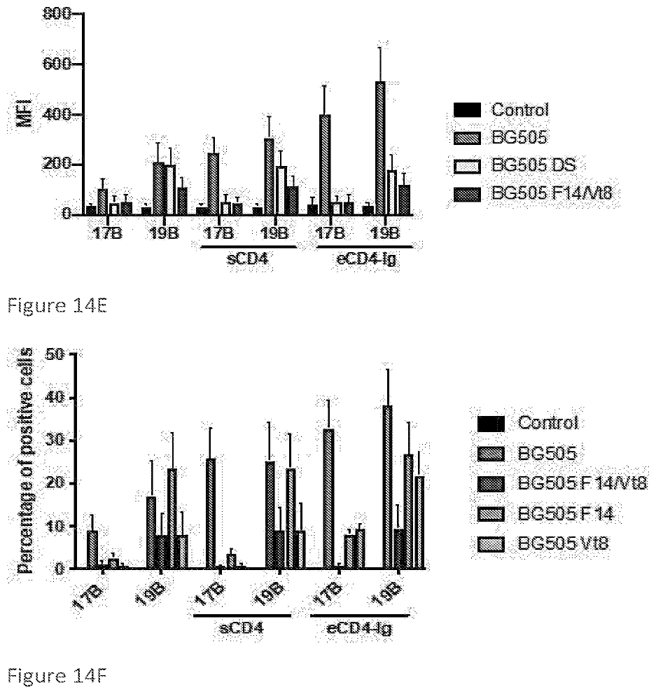

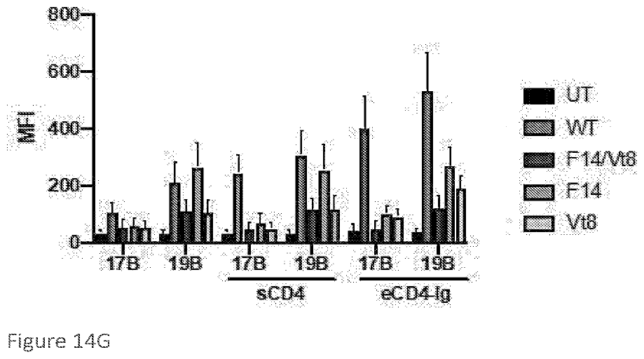

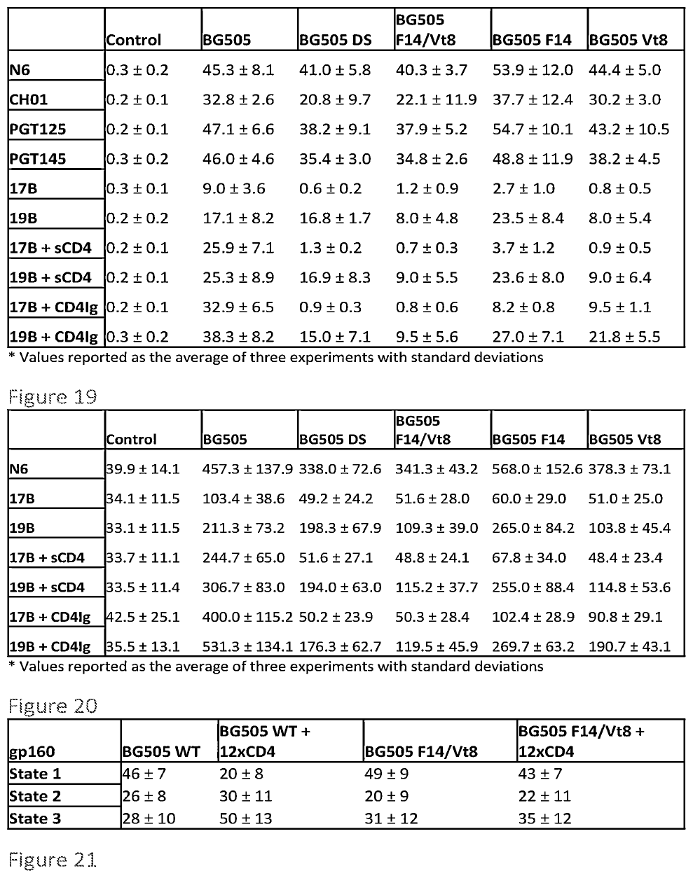

In the assay using cell surface Env gp160s, BG505, a previously engineered BG505 stabilized design termed BG505 DS¹⁸, and BG505 F14/Vt8, BG505 F14, and BG505 Vt8 were first tested for binding to bnAbs N6, CH01, PGT125, and PGT145 targeting CD4bs, V1/V2, glycan-V3, and trimer apex epitopes, respectively. Binding to cell surface expressed Env trimer was assessed via flow cytometry to determine the percentage of positive cells and mean fluorescence intensities (MFI) for bnAb binding (FIG. 13). The percentage of cells testing positive for binding to each bnAb were similar in each construct with the exception of BG505 DS binding to CH01, which displayed a marked reduction in cells positive for CH01 binding (FIG. 6A, FIG. 14A, FIG. 19). We next examined binding of non-bnAbs 19B and 17B, which target the open state V3-tip and bridging sheet epitopes, respectively. Binding to these open state preferring antibodies was tested in the presence and absence of sCD4 or potently neutralizing eCD4-Ig, a coreceptor-mimetic peptide fused eCD4-Ig³³. In order to ensure comparable levels of Env surface expression between constructs, the near-pan neutralizing bnAb N6³⁴ was used as a benchmark yielding comparable binding for all constructs tested with MFIs of 457.3±137.9, 338.0±72.6, 341.3±43.2, 568.0±152.6, and 378.3±73.1 for BG505, BG505 DS, BG505 F14/Vt8, BG505 F14, and BG505 Vt8, respectively (FIGS. 14B and C, FIG. 20).

Pairwise percentage positive cell comparison of each construct in each replicate experiment demonstrated a consistent reduction of intrinsic exposure of the 17B open state epitope in both the BG505 DS and BG505 F14/Vt8 stabilized gp160 trimers as compared to BG505 gp160 (FIG. 6B, FIGS. 14D and E, FIG. 19). Intrinsic exposure of the V3 tip targeting 19B epitope was relatively similar between BG505 and BG505 DS gp160s while the BG505 F14/Vt8 gp160 displayed a consistent ˜2-fold reduction in 19B epitope exposure relative to BG505 gp160 (FIG. 6B, FIGS. 14D and E, FIG. 19). On incubating the cells with either sCD4 or eCD4-Ig, distinct increases in both the 17B and 19B percentage of positive cells were observed for BG505 gp160, as expected (FIG. 6B, FIGS. 14D and E, FIG. 19).

Conversely, binding to these CD4-induced epitope-targeting antibodies was not observed for BG505 DS or F14/Vt8 stabilized gp160 trimers in the presence of either sCD4 or eCD4-Ig (FIG. 6B, FIGS. 14D and E, FIG. 19). Triggering of the 17B epitope was not observed in BG505 F14 or BG505 Vt8 gp160s in the presence of sCD4, although 19B epitope exposure was observed in each (FIGS. 14F and G, FIG. 19). Additionally, both 17B and 19B epitope exposure was observed in the presence of eCD4-Ig (FIGS. 14F and G, FIG. 19). These results showed that the F14, Vt8, and F14/Vt8 mutations allowed presentation of key bnAb epitopes and that the combined F14/Vt8 mutations were necessary for the minimization of intrinsic non-bnAb epitope exposure and the prevention of CD4 induced rearrangement of the surface expressed gp160 Env.

We also used the smFRET assay developed by Munro et. al¹⁷, to examine the conformational landscape and CD4-induced effects on the virion bound F14/Vt8 Env as compared to the BG505 Env. Specifically, we examined the FRET distributions of the BG505 and F14/Vt8 BG505 Envs to determine a) whether the mutations alter the unliganded FRET distribution relative to BG505 Env and b) whether the mutations prevent dodecameric CD4 (sCD4D1D2-Igαtp) induced rearrangements. smFRET studies previously revealed that native Env on virus predominantly resides in in a conformational state termed State 1 but has spontaneous access to two more conformational states, termed states 2 and 3. In response to CD4 binding, wild-type Env opens into State 3 through one necessary intermediate (State 2).^{17,35}

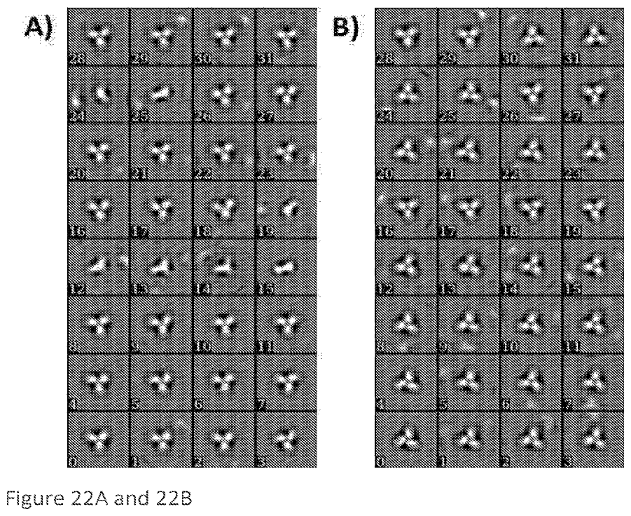

Comparison of the unbound Envs indicated that both unliganded Envs displayed similar distributions, with the predominant State 1 peak at low FRET, yielding State 1 occupancies of ˜46% and ˜49% for the BG505 and F14/Vt8 Envs, respectively (FIGS. 6C and D, FIG. 21). Both trimers also displayed similar occupancies of State 2 and State 3. Addition of CD4 resulted in a shift in the distribution for the BG505 Env toward increased high and mid-FRET states corresponding to the asymmetric intermediate State 2 configuration and the open State 3 configuration, respectively (FIGS. 6C and D, FIG. 21). Unlike the BG505 Env, however, the F14/Vt8 Env did not respond to addition of dodecameric CD4, displaying the same relative distribution of the three FRET states in the presence of CD4 as were seen in the absence of CD4, and consistent with the observed resistance to CD4-induced changes in the BG505 F14/Vt8 SOSIP Env (FIGS. 6C and D, FIG. 21). Together, results for the cell and viral membrane associated gp160 Env F14/Vt8 mutant trimer are consistent with observations for these mutations in the soluble SOSIP trimer indicating the mutations designed to disable Env allostery effectively blocked CD4-induced rearrangements while maintaining efficient bnAb interaction.

### Discussion

The HIV-1 Env is an intricate conformational machine that propagates receptor-mediated structural changes from a neutralization-resistant closed conformation to a fusion-competent open conformation that exposes immunodominant epitopes for non-neutralizing antibodies. The closed conformation of the HIV-1 Env is of interest for vaccine design since it presents the epitopes for broadly neutralizing antibodies, and the expectation is that effective presentation of the native, closed conformation of the Env by vaccination is essential to elicit broadly neutralizing antibodies. Indeed, many studies have succeeded in stabilizing the closed conformation of the HIV-1 Env both in the soluble Env format, as well on the cell surface, with varying levels of success in eliciting autologous and heterologous neutralization.¹¹⁻¹⁴

In this study we combined structural and mechanistic information to construct a mutant Env that is no longer responsive to triggering by the CD4 receptor. Two strategies were employed, each aimed at shifting the equilibrium of Env dynamics towards its closed state. The first strategy that led to the F14 series of mutations identified the conserved W571 as a conformational switch and effectively disabled a communication network that relied on the movement of topological layers 1 and 2 to transmit structural changes from the CD4 binding site. The second path aimed to prevent V3 exposure via mutation of buried hydrophilic to hydrophobic residues in order to prevent the infiltration of water into the space between V3 and V1/V2. Together, the findings presented here indicated close coupling of sCD4 induced internal rearrangements in gp120 and the N-terminal portion of the gp41 three-helix bundle.

While these designs were made and tested in the context of the soluble SOSIP env, the observed effects translated to the Env on the native virion surface, suggesting that despite the differences between the native Env and the engineered SOSIPs, an allosteric network that was common to both formats was disabled by the mutations.³⁶ Indeed, the structure of a PGT151-bound full length Env trimer that was purified by detergent solubilization of cell surface expressed gp160 demonstrated contacts between the topological layers of gp120 and the gp41 three-helix bundle consistent with this proposal.³⁷ Differences between this structure and a full-length virion-bound Env outside of this region notwithstanding, we propose a sequential mechanism by which the Env transitions from the prefusion closed state to a fully open, fusion-competent state through a series of sequential steps (FIG. 7D). Beginning from a closed state in which the trimer apex and topological layers surround the gp41 three-helix bundle³⁸, engagement of a single CD4 induces β20-β21 loop rearrangement and is associated with Env triggering via residue I432 in β20-β21 and residue L193 in V1/V2.²³

Instability in V1/V2 associated with these changes would allow V3 exposure^{27,39} and initial apex dissociation of a single gp120 protomer from the gp41 three-helix bundle²⁴. Internal configurational changes in V1/V2 could then propagate to layers-1 and 2, thereby releasing W571, thus lowering the potential energy barrier to full gp120-gp41 three-helix bundle dissociation resulting in a single gp120 open, asymmetric trimer configuration.³⁵ Indeed, viruses with substitution of W571 are replication deficient and non-infectious due to abolished membrane fusion activity despite effective cell surface expression, processing, and retention of CD4 binding, demonstrating the importance of this residue in downstream CD4-induced changes.^{40,41} Absent a stable trimeric apex interface, the remaining two closed state protomers could then decay toward the open state, potentially assisted by CD4 quaternary contacts.^{20,38,42} Together, with the mechanistic insights provided by the observed structural rearrangements in the SOSIP Env, the results from this study indicate direct manipulation of the Env allosteric network allows for fine-tuned conformational control of Env structure that can assist in the development and refinement of the next generation of Env vaccine immunogens.

### Methods

### Recombinant HIV-1 Envelope SOSIP gp140 Production

Antibodies and antibody Fabs were produced as described previously.⁴³ BG505 N332 SOSIP gp140 envelopes were expressed as previously described with minor modifications.⁴⁴ Envelope production was performed with Freestyle293 cells (Invitrogen). On the day of transfection, Freestyle293 were diluted to 1.25×10⁶ cells/mL with fresh Freestyle293 media up to 1L total volume. The cells were co-transfected with 650 μg of SOSIP expressing plasmid DNA and 150 μg of furin expressing plasmid DNA complexed with 293Fectin (Invitrogen). On day 6cell culture supernatants were harvested by centrifugation of the cell culture for 30 min at 3500 rpm. The cell-free supernatant was filtered through a 0.8 μm filter and concentrated to less than 100 mL with a single-use tangential flow filtration cassette and 0.8 μm filtered again. Trimeric Env protein was purified with monoclonal antibody PGT145 affinity chromatography. PGT145-coupled resin was packed into Tricorn column (GE Healthcare) and stored in PBS supplemented with 0.05% sodium azide. Cell-free supernatant was applied to the column at 2 mL/min using an AKTA Pure (GE Healthcare), washed, and protein was eluted off of the column with 3M MgCl₂. The eluate was immediately diluted in 10 mM Tris pH8, 0.2 μm filtered, and concentrated down to 2 mL for size exclusion chromatography. Size exclusion chromatography was performed with a Superose6 16/600 column (GE Healthcare) in 10 mM Tris pH8, 500 mM NaCl. Fractions containing trimeric HIV-1 Env protein were pooled together, sterile-filtered, snap frozen, and stored at −80° C.

### Biolayer Interferometry

Cell supernatant mAb binding and dose response curves were obtained using biolayer interferometry (BLI; OctetRed96, FortéBio). Antibodies were immobilized on anti-Human IgG Fc capture (AHC; FortéBio) sensor tips via immersion in 20 μg/ml mAb in phosphate buffered saline (PBS) for 300 s followed by washing in PBS for 60 s at 1000 rpm. For the cell supernatant assays, the mAb captured sensor tips were then immersed in 200 μl of the transfection supernatant for 400 s at 1000 rpm for the association phase after which the sensor tips were immersed in PBS for 600 s for the dissociation phase. The sensor tips were regenerated using glycine pH 2.0 for an immersion time of 20 s between measurements. For the dose response experiments, the sensor tips were immersed in the SOSIP-containing wells for 180 s for the association and 60 s for dissociation phase using a shake speed of 1000 rpm beginning from the lowest SOSIP concentration. Regeneration was not performed between measurements of increase SOSIP concentration. A longer, final dissociation phase of 600 s was measured for the highest concentration SOSIP containing well. Non-specific binding was accounted for via subtraction of sensorgrams obtained using the anti-Flu Hemagglutinin Ab82 control mAb. Reported binding corresponds to values and the end of the association phase. Data was evaluated using the Octet Data Analysis 10.0 software (FortéBio) with dose response curves fitted in GraphPad Prism using the one site specific binding model.

### Surface Plasmon Resonance

Triggering of SOSIP gp140 Envs by soluble CD4 (sCD4) was monitored via surface Plasmon resonance and was performed on a BIAcore 3000 instrument (GE Healthcare). Antibodies were immobilized using direct immobilization using amine coupling on CM3 sensor chips (GE Healthcare) at ˜5000 RU. The SOSIP concentration in the presence and absence of a 1:3 mixture of sCD4 was 200 nM. Samples were injected over at a rate of 30 μl/min for a total of 90 μul using the high performance kinject injection mode with a dissociation phase of 600 s over four flow cells containing 17B, 19B, VRC01, or the Ab82 control. The chip surface was regenerated between measurements using two injections of 20 μl of glycine pH 2.0 at a flow rate of 50 μl/min. The resulting response curves were processed using the BIAevaluation 4.1 (GE Healthcare) software using a double reference subtraction. Reported response values were determined taking the average response from 170-175 seconds after the start of the injection.

### Thermal Denaturation

Thermal denaturation experiments were performed using the NanoDSC platform (TA Instruments). Samples were dialyzed into HEPES buffered saline (HBS; 10 mM HEPES, 150 mM NaCl, pH 7.4), diluted in dialysate to 0.2 to 0.4 mg/ml, and degassed for 15 minutes. Following condition of the DSC cells in dialysate, samples were loaded and heated from 20° C. to 100° C. at 3 atm of pressure at a rate of 1° C./min using the dialysate as a reference. The obtained denaturation profiles were buffer subtracted and base line corrected using a 6^{th}-order polynomial using the NanoAnalyze software (TA Instruments). The reported Tₘₐₓ corresponds to the average maximum observed heat capacity from three independent measures.

### Cryo-FM Sample Preparation

The BG505 F14 and F14/Vt8 SOSIP trimers complexes were prepared using a stock solution of 2 mg/ml trimer incubated with a six-fold molar excess of VRC01 or VRC03 and 10-1074, respectively. To prevent interaction of the trimer complexes with the air-water interface during vitrification, the samples were incubated in 0.085 mM n-dodecyl β-D-maltoside (DDM). Samples were applied to plasma-cleaned QUANTIFOIL holey carbon grids (EMS, R1.2/1.3 Cu 300 mesh) followed by a 30 second adsorption period and blotting with filter paper. The grid was then plunge frozen in liquid ethane using an EM GP2 plunge freezer (Leica, 90-95% relative humidity).

### Cryo-EM Data Collection

Cryo-EM imaging was performed on a FEI Titan Krios microscope (Thermo Fisher Scientific) operated at 300 kV. Data collection images were acquired with a Falcon 3EC Direct Electron Detector operated in counting mode with a calibrated physical pixel size of 1.08 Å with a defocus range between −1.0 and −3.5 μm using the EPU software (Thermo Fisher Scientific). No energy filter or Cₛ corrector was installed on the microscope. The dose rate used was ˜0.8 e⁻/Å²·s to ensure operation in the linear range of the detector. The total exposure time was 60 s, and intermediate frames were recorded every 2 s giving an accumulated dose of ˜42 e⁻/Å² and a total of 30 frames per image. A total of 2,350 images for BG505-F14-SOSIP and 2,060 images for BG505-F14/Vt8-SOSIP were collected over two days, respectively.

### Data Processing

Cryo-EM image quality was monitored on-the-fly during data collection using automated processing routines. Initial data processing was performed within cryoSPARC²⁹ including particle picking, multiple rounds of 2D classification, ab initio reconstruction, homogeneous map refinement and non-uniform map refinement, yielding 3.5 Å and 3.7 Å maps for the BG505-F14-SOSIP and the BG505-F14/Vt8-SOSIP complexes, respectively. Further processing was done outside of cryoSPARC as described next. Movie frame alignment was carried out using UNBLUR⁴⁵, and CTF determination using CTFFIND4⁴⁶. Particles were picked automatically using a Gaussian disk of 90 Å in radius as the search template. For the BG505-F14-SOSIP dataset, 1,518,046 particles were picked from 2,350 micrographs, extracted using a binning factor of 2 and subjected to 8 rounds of refinement in cisTEM⁴⁷, using an ab-initio model generated with cryoSPARC²⁹. The distribution of scores assigned to each particle by cisTEM showed a clear bi-modal distribution and only particles in the group containing the higher scores were selected for further processing (FIG. 10). This subset of 77,632 particles was re-extracted without binning and subjected to 10 rounds of local refinement followed by 5 additional rounds using a shape mask generated with EMAN2⁴⁸. Per-particle CTF refinement was then conducted until no further improvement in the FSC curve was observed. At this point, particle frames were re-extracted from the raw data and subjected to per-particle motion correction using all movie frames and applying a data-driven dose weighting scheme as described previously.⁴⁹ The per-particle refinement procedure was iterated two additional times using the newly generated map as a reference and at that point no further improvement was observed. The resolution of the final map was 3.0 Å measured according to the 0.143-cutoff FSC criteria. A b-factor of −120 Å² was applied to the reconstruction for purposes of visualization. For the BG505-F14/Vt8-SOSIP dataset, 869,323 particles were picked from 2,060 micrographs, and a subset of 84,378 particles was used for further local refinement using a similar strategy as that used to process the BG505-F14-SOSIP dataset. The estimated resolution for the final map in this case was 2.9 Å according to the 0.143-cutoff FSC criteria.

### Cryo-EM Structure Fitting

Structure fitting of the cryo-EM maps was performed in Chimera⁵⁰ using the gp120 and gp41 segments from PDB ID 5CEZ (chains G and B, respectively) with F14 and Vt8 mutations added using PyMol. Coordinates for VRC01, VRC03, and 10-1074 were obtained from PDB ID 3NGB (chains H and L), PDB ID 3SE8 (chains H and L), and PDB ID 5T3Z (chains H and L), respectively. Initial coordinate refinement was performed using Rosetta.⁵¹ The best fit from 110 models was then iteratively with manual refinement in Coot⁵² followed by real-space refinement in Phenix.⁵³ Structure fit and map quality statistics were determined using MolProbity⁵⁴ and EMRinger⁵⁵, respectively. Structure and map analyses were performed using a combination of PyMol⁵⁶ and Chimera.

### Vector Based Structure Analysis

Centroids for the vectors in the analysis included a K46-K490 Cα centroid, W571 and W596 c-αs, c-αs of gp120 excluding variable loops the V1/V1 region residues, and the N-and C-termni, and a V1/V2+V3 c-α centroid Vectors between these reference positions were generated and included a projection of the W596 to K46-K490 centroid vector on to the W596 to W571 vector. Angles, distances, and dihedrals between these vectors were then compiled for a set of available crystal and cryo-EM structures with resolutions better than 4.5 Å (PDB IDs for closed state structures 4TVP⁵⁷, 4ZMJ¹⁸, 5ACO⁵⁸, 5CEZ³¹, 5CJX⁵⁹, 5D9Q⁶⁰, 5FYJ⁶¹, 5FYK⁶¹, 5FYL⁶¹, 5T3X⁶², 5T3Z⁶², 5U7M²⁶, 5U7O²⁶, 5UTF¹³, 5V7J⁶³, 5V8L⁶⁴, 5V8M⁶⁴, 6CDE³², 6CDI³², 6CH7⁶⁵, 6CH8⁶⁵, 6CUE⁶⁶, 6CUF⁶⁷, and 6DE7¹²; Open and partially open state structures 5THR¹⁹ chains A and E, 5VN3²⁰ chains A and G, 5VN8⁵⁹ chains A and G, 6CM3²⁴ chains A and E, and 6EDU²⁴ chains A and D; PGT151 bound structures, 5FUU⁶⁸ chains A and B, C and D, and E and F, 6DCQ³⁷ chains A and B, C and D, and E and F, 6MAR⁶⁹ chains A and B, C and D, and E and F) in addition to a single CD4 bound SOSIP trimer (PDB ID 5U1F⁴² chains A and B, C and E, and D and F), as well as the BG505 F14 and F14/Vt8 SOSIP structures determined in this study. A single protomer was analyzed for each structure with the exception of PGT151 and single CD4 bound structures for which each protomer was analyze. Vector based structural analysis was performed using the VMD⁷⁰ Tcl interface. Principal component analysis of the resulting vectors, angles, and torsions was performed using R⁷¹.

### Cell-Surface Expressed Env gp160 Antigenicity

FreeStyle 293F cells (ThermoFisher, catalog #R79007) (1×10⁶ cells/ml, 0.5 ml per well of a 12-well plate) were transfected using a mixture of 1 μg HIV Env gp160s DNA in 75 μl jetPRIME buffer (Polyplus-transfection) with 2 μl jetPRIME transfection reagent (Polyplus-transfection) following the manufacturer's protocol. Transfected cells were cultured in Freestyle 293 Expression Medium (Invitrogen Inc.) at 120 rpm for 48 hours before flow cytometry staining.

293F cells were counted using trypan blue and then were rinsed with PBS containing 1% BSA, pelleted at 250×g for 3 min. Cells were incubated with wither sCD4 or eCD4-Ig³³ at a final concentration of 10 μg/mL for 20 min at 4° C. Human anti-HIV Env Abs at a final concentration of 10 μg/mL were used to stain 0.4×10⁶ cells per well in 40 μL PBS containing 1% BSA of in V bottom 96-well plates, 30 min at RT in the dark. Cells were then washed once with 200 μL PBS containing 1% BSA and incubated with PE conjugated Goat F(ab′)2 Anti-Human IgG-(Fab′)2 secondary antibody (abcam, Cambridge, MA) at a final concentration of 2.5 μg/ml in 100 μL PBS containing 1% BSA per well. After 30 min incubation at 4° C. in the dark, cells were washed once, and stained with 200 μL aqua viability dye (1:1000 in PBS) for 20 min at RT in the dark, then washed twice with PBS containing 1% BSA. Flow cytometric data were acquired on a LSRII using FACSDIVA software (BD Biosciences) and were analyzed with FlowJo software (FlowJo). FACSDIVA software (BD Biosciences) and were analyzed with FlowJo software.

### Preparation and smFRET Analysis of Dye-Labeled HIV-1_{BG505} Virus Env

Dye-labeled wild-type and mutant F14Vt8 HIV-1_{BG505} virus Env were prepared and imaged as previously described.³⁶ Briefly, peptides-tagged Envs in the context of full-length virus were constructed by introducing two labeling tag peptides (GQQQLG (SEQ ID NO: 9); GDSLDMLEWSLM (SEQ ID NO: 10)) into variable loops V1 and V4 of gp120 subunit on both wild-type and F14Vt8 BG505 Env in the context of a replication competent clade A virus carrying the Q23 as the backbone. Wild-type and mutant BG505 virus Env for smFRET imaging was prepared by co-transfecting HEK293 cells with mixed plasmids consisting of a 40:1 ratio of replication-incompetent (RT deleted) wild-type vs. peptides-tagged HIV-1_{BG505} virus Env. The viruses were harvested 40-hour post-transfection, filtered, concentrated, and labeled with donor dye Cy3B(3S)-cadaverine (0.5 μM) and acceptor dye LD650-CoA (0.5 μM, Lumidyne Technologies) in the presence of transglutaminase (0.65 μM, Sigma Aldrich) and AcpS (5 μM) at room temperature overnight. PEG2000-biotin (0.02 mg/ml, Avanti Polar Lipids) was then added to above labeling solutions the next day and incubated for 30 minutes prior to purification. Labeled viruses were further purified by ultracentrifugation at 40,000 rpm over a 6%-18% Optiprep (Sigma Aldrich) gradient for 1 hour.

Dye-labeled virus Envs were immobilized on a streptavidin-coated quartz microscopy slide for smFRET imaging. smFRET movies were acquired on a prism-based total internal reflection fluorescence microscope, and FRET traces were extracted and analyzed by using a customized software package based on Lab View and Mathworks.⁷² The evanescent field generated by a 532 nm CW laser (Opus, Laser Quantum) was used to excite donor dye. Fluorescence from both donor and acceptor dyes that are labeled on HIV-1_{BG505} virus Env was firstly collected, then spectrally split, and later simultaneously detected by two synchronized sCMOS cameras (ORCA-Flash4.0v2, Hamamatsu). The dynamics of fluorescently labeled Env on virus was traced by monitoring fluorescence signals with 40 milliseconds time-resolution for 80 seconds in imaging buffer containing 50 mM Tris pH 7.4, 50 mM NaCl, a cocktail of triplet-state quenchers, and oxygen scavenger consisted of 2 mM protocatechuic acid with 8 nM protocatechuic-3,4-dioxygenase. In the case of ligands binding experiments, fluorescently labeled wild-type or F14Vt8 mutant virus Env was pre-incubated with 10 μg/ml sCD4D1D2-Igαtp (12xCD4) for 30 min at room temperature before imaging.

FRET (presented as efficiency/value) traces in real time were derived from fluorescence signals traces based on the equation FRET=I_{A}/(γI_{D}+I_{A}), where I_{D} and I_{A} are the fluorescence intensities of donor (I_{D}) and acceptor (I_{A}), and γ is the correlation ecoefficiency. FRET traces that pass through the stringent filters of signal-to-noise (S/N) ratio as well as anti-correlated features between I_{D} and I_{A} were used to build FRET histograms. On the basis of the observations from original FRET traces and the idealization by hidden Markov modeling, FRET histograms were further fitted into the sum of three Gaussian distributions. Relative state occupancy of each FRET state was estimated as the area under each Gaussian curve, where standard errors was calculated from thousands of individual data points that corresponds to the indicated number (N) of molecules in FRET histogram.

### BIBLIOGRAPHY FOR EXAMPLES

Tran, E. E. H. et al. Structural Mechanism of Trimeric HIV-1 Envelope Glycoprotein Activation. PLOS Pathogens 8, e1002797, doi:10.1371/journal.ppat.1002797 (2012).
2 Ward, A. B. & Wilson, I. A. The HIV-1 envelope glycoprotein structure: nailing down a moving target. Immunological Reviews 275, 21-32 (2017).
3 Ward, A. B. & Wilson, I. A. Insights Into the Trimeric HIV-1 Envelope Glycoprotein Structure. Trends in biochemical sciences 40, 101-107, doi:10.1016/j.tibs.2014.12.006 (2015).
4 Chan, D. C. & Kim, P. S. HIV Entry and Its Inhibition. Cell 93, 681-684, doi:https://doi.org/10.1016/S0092-8674 (00)81430-0 (1998).
5 Weissenhorn, W., Dessen, A., Harrison, S. C., Skehel, J. J. & Wiley, D. C. Atomic structure of the ectodomain from HIV-1 gp41. Nature 387, 426-430, doi:10.1038/387426a0 (1997).
6 Shaik, M. M. et al. Structural basis of coreceptor recognition by HIV-1 envelope spike. Nature 565, 318-323, doi:10.1038/s41586-018-0804-9 (2019).
7 Kwong, P. D. et al. Structure of an HIV gp120 envelope glycoprotein in complex with the CD4 receptor and a neutralizing human antibody. Nature 393, 648-659 (1998).
8 Harrison, S. C. Viral membrane fusion. Nature Structural & Amp; Molecular Biology 15, 690, doi:10.1038/nsmb.1456 (2008).
9 Sanders, R. W. et al. A Next-Generation Cleaved, Soluble HIV-1 Env Trimer, BG505 SOSIP.664 gp140, Expresses Multiple Epitopes for Broadly Neutralizing but Not Non-Neutralizing Antibodies. PLOS Pathogens 9, e1003618, doi:10.1371/journal.ppat.1003618(2013).
10 de Taeye, Steven W. et al. Immunogenicity of Stabilized HIV-1 Envelope Trimers with Reduced Exposure of Non-neutralizing Epitopes. Cell 163, 1702-1715, doi:https://doi.org/10.1016/j.cell.2015.11.056 (2015).
11 He, L. et al. HIV-1 vaccine design through minimizing envelope metastability. Science advances 4, eaau6769-eaau6769, doi:10.1126/sciadv.aau6769 (2018).
12 Zhang, P. et al. Interdomain Stabilization Impairs CD4 Binding and Improves Immunogenicity of the HIV-1 Envelope Trimer. Cell Host & Microbe 23, 832-844.e836,doi:https://doi.org/10.1016/j.chom.2018.05.002 (2018).
13 Chuang, G.-Y. et al. Structure-Based Design of a Soluble Prefusion-Closed HIV-1 Env Trimer with Reduced CD4 Affinity and Improved Immunogenicity. Journal of Virology 91, doi:10.1128/JVI.02268-16 (2017).
14 Torrents de la Peña, A. et al. Improving the Immunogenicity of Native-like HIV-1 Envelope Trimers by Hyperstabilization. Cell reports 20, 1805-1817, doi:10.1016/j.celrep.2017.07.077 (2017).
15 Munro, J. B. & Mothes, W. Structure and Dynamics of the Native HIV-1 Env Trimer. Journal of Virology 89, 5752-5755 (2015).
16 Pugach, P. et al. A Native-Like SOSIP.664 Trimer Based on an HIV-1 Subtype B env Gene. Journal of Virology 89, 3380-3395, doi:10.1128/JVI.03473-14 (2015).
17 Munro, J. B. et al. Conformational dynamics of single HIV-1 envelope trimers on the surface of native virions. Science 346, 759, doi:10.1126/science.1254426 (2014).
18 Kwon, Y. D. et al. Crystal structure, conformational fixation, and entry-related interactions of mature ligand-free HIV-1 Env. Nature structural & molecular biology 22, 522-531, doi:10.1038/nsmb.3051 (2015).
19 Wang, H. et al. Cryo-EM structure of a CD4-bound open HIV-1 envelope trimer reveals structural rearrangements of the gp120 V1V2 loop. Proceedings of the National Academy of Sciences 113, E7151-E7158, doi:10.1073/pnas.1615939113 (2016).
20 Ozorowski, G. et al. Open and closed structures reveal allostery and pliability in the HIV-1 envelope spike. Nature 547, 360-363, doi:10.1038/nature23010
http://www.nature.com/nature/journal/v547/n7663/abs/nature23010.html#supplementary-information (2017).
21 Pancera, M. et al. Structure of HIV-1 gp120 with gp41-interactive region reveals layered envelope architecture and basis of conformational mobility. Proceedings of the National Academy of Sciences 107, 1166, doi:10.1073/pnas.0911004107 (2010).
22 Finzi, A. et al. Topological Layers in the HIV-1 gp120 Inner Domain Regulate gp41 Interaction and CD4-Triggered Conformational Transitions. Molecular cell 37, 656-667, doi:10.1016/j.molcel.2010.02.012 (2010).
23 Herschhorn, A. et al. The β20-β21 of gp120 is a regulatory switch for HIV-1 Env conformational transitions. Nature Communications 8, 1049, doi:10.1038/s41467-017-01119-w (2017).
24 Wang, H., Barnes, C. O., Yang, Z., Nussenzweig, M. C. & Bjorkman, P. J. Partially Open HIV-1 Envelope Structures Exhibit Conformational Changes Relevant for Coreceptor Binding and Fusion. Cell Host & Microbe 24, 579-592.e574, doi:https://doi.org/10.1016/j.chom.2018.09.003 (2018).
25 Meanwell, N. A. et al. Inhibitors of HIV-1 Attachment: The Discovery and Development of Temsavir and its Prodrug Fostemsavir. Journal of Medicinal Chemistry 61, 62-80, doi:10.1021/acs.jmedchem.7b01337 (2018).
26 Pancera, M. et al. Crystal structures of trimeric HIV envelope with entry inhibitors BMS-378806 and BMS-626529. Nature Chemical Biology 13, 1115,doi:10.1038/nchembio.2460
https://www.nature.com/articles/nchembio.2460#supplementary-information (2017).
27 Zolla-Pazner, S. et al. Structure/Function Studies Involving the V3 Region of the HIV-1 Envelope Delineate Multiple Factors That Affect Neutralization Sensitivity. Journal of virology 90, 636-649, doi:10.1128/JVI.01645-15 (2015).
28 Kulp, D. W. et al. Structure-based design of native-like HIV-1 envelope trimers to silence non-neutralizing epitopes and eliminate CD4 binding. Nature Communications 8, 1655, doi:10.1038/s41467-017-01549-6 (2017).
29 Punjani, A., Rubinstein, J. L., Fleet, D. J. & Brubaker, M. A. cryoSPARC: algorithms for rapid unsupervised cryo-EM structure determination. Nature Methods 14, 290, doi:10.1038/nmeth.4169
https://www.nature.com/articles/nmeth.4169#supplementary-information (2017).
30 Lemmin, T., Soto, C., Stuckey, J. & Kwong, P. D. Microsecond Dynamics and Network Analysis of the HIV-1 SOSIP Env Trimer Reveal Collective Behavior and Conserved Microdomains of the Glycan Shield. Structure 25, 1631-1639.e1632, doi:10.1016/j.str.2017.07.018 (2017).
31 Garces, F. et al. Affinity Maturation of a Potent Family of HIV Antibodies Is Primarily Focused on Accommodating or Avoiding Glycans. Immunity 43, 1053-1063, doi:10.1016/j.immuni.2015.11.007 (2015).
32 Xu, K. et al. Epitope-based vaccine design yields fusion peptide-directed antibodies that neutralize diverse strains of HIV-1. Nature Medicine 24, 857-867,doi:10.1038/s41591-018-0042-6 (2018).
33 Gardner, M. R. et al. AAV-expressed eCD4-Ig provides durable protection from multiple SHIV challenges. Nature 519, 87-91, doi:10.1038/nature14264 (2015).
34 Huang, J. et al. Identification of a CD4-Binding-Site Antibody to HIV that Evolved Near-Pan Neutralization Breadth. Immunity 45, 1108-1121, doi:10.1016/j.immuni.2016.10.027 (2016).
35 Ma, X. et al. HIV-1 Env trimer opens through an asymmetric intermediate in which individual protomers adopt distinct conformations. eLife 7, e34271, doi:10.7554/eLife.34271 (2018).
36 Lu, M. et al. Associating HIV-1 envelope glycoprotein structures with states on the virus observed by smFRET. Nature 568, 415-419, doi:10.1038/s41586-019-1101-y (2019).
37 Rantalainen, K. et al. Co-evolution of HIV Envelope and Apex-Targeting Neutralizing Antibody Lineage Provides Benchmarks for Vaccine Design. Cell Reports 23, 3249-3261, doi:https://doi.org/10.1016/j.celrep.2018.05.046 (2018).
38 Liu, J., Bartesaghi, A., Borgnia, M. J., Sapiro, G. & Subramaniam, S. Molecular architecture of native HIV-1 gp120 trimers. Nature 455, 109-113, doi:10.1038/nature07159 (2008).
39 Powell, R. L. R. et al. Plasticity and Epitope Exposure of the HIV-1 Envelope Trimer. Journal of Virology 91, e00410-00417, doi:10.1128/JVI.00410-17 (2017).
40 Cao, J. et al. Effects of amino acid changes in the extracellular domain of the human immunodeficiency virus type 1 gp41 envelope glycoprotein. Journal of virology 67, 2747-2755 (1993).
41 Mo, H. et al. Conserved residues in the coiled-coil pocket of human immunodeficiency virus type 1 gp41 are essential for viral replication and interhelical interaction. Virology 329, 319-327, doi:https://doi.org/10.1016/j.virol.2004.08.025 (2004).
42 Liu, Q. et al. Quaternary contact in the initial interaction of CD4 with the HIV-1 envelope trimer. Nature Structural & Amp; Molecular Biology 24, 370, doi:10.1038/nsmb.3382
https://www.nature.com/articles/nsmb.3382#supplementary-information (2017).
43 Saunders, K. O. et al. Vaccine Elicitation of High Mannose-Dependent Neutralizing Antibodies against the V3-Glycan Broadly Neutralizing Epitope in Nonhuman Primates. Cell Reports 18, 2175-2188, doi:https://doi.org/10.1016/j.celrep.2017.02.003 (2017).
44 Saunders, K. O. et al. Vaccine Induction of Heterologous Tier 2 HIV-1 Neutralizing Antibodies in Animal Models. Cell Reports 21, 3681-3690,doi:https://doi.org/10.1016/j.celrep.2017.12.028 (2017).
45 Grant, T. & Grigorieff, N. Measuring the optimal exposure for single particle cryo-EM using a 2.6 Å reconstruction of rotavirus VP6. eLife 4, e06980, doi:10.7554/eLife.06980 (2015).
46 Rohou, A. & Grigorieff, N. CTFFIND4: Fast and accurate defocus estimation from electron micrographs. Journal of Structural Biology 192, 216-221, doi:https://doi.org/10.1016/j.jsb.2015.08.008 (2015).
47 Grant, T., Rohou, A. & Grigorieff, N. cisTEM, user-friendly software for single-particle image processing. eLife 7, e35383, doi:10.7554/eLife.35383 (2018).
48 Tang, G. et al. EMAN2: An extensible image processing suite for electron microscopy. Journal of Structural Biology 157, 38-46, doi:http://dx.doi.org/10.1016/j.jsb.2006.05.009 (2007).
49 Bartesaghi, A. et al. Atomic Resolution Cryo-EM Structure of β-Galactosidase. Structure 26, 848-856.e843, doi:https://doi.org/10.1016/j.str.2018.04.004 (2018).
50 Pettersen, E. F. et al. UCSF Chimera—A visualization system for exploratory research and analysis. Journal of Computational Chemistry 25, 1605-1612, doi:10.1002/jcc.20084 (2004).
51 Wang, R. Y.-R. et al. Automated structure refinement of macromolecular assemblies from cryo-EM maps using Rosetta. eLife 5, e17219, doi:10.7554/eLife.17219 (2016).
52 Features and development of Coot.—Acta crystallographica. Section D, Biological crystallography—66,—486-501, doi:—(2010).
53 Afonine, P. V. et al. Real-space refinement in PHENIX for cryo-EM and crystallography. Acta Crystallographica Section D 74, 531-544, doi:10.1107/S2059798318006551 (2018).
54 Chen, V. B. et al. MolProbity: all-atom structure validation for macromolecular crystallography. Acta Crystallogr D Biol Crystallogr 66, 12-21, doi:10.1107/S0907444909042073 (2010).
55 Barad, B. A. et al. EMRinger: side chain-directed model and map validation for 3D cryo-electron microscopy. Nature Methods 12, 943, doi:10.1038/nmeth.3541
https://www.nature.com/articles/nmeth.3541#supplementary-information (2015).
56 Schrodinger, L. The PyMOL Molecular Graphics System. (2015).
57 Pancera, M. et al. Structure and immune recognition of trimeric pre-fusion HIV-1 Env. Nature 514, 455, doi:10.1038/nature13808
https://www.nature.com/articles/nature13808#supplementary-information (2014).
58 Lee, Jeong H., de Val, N., Lyumkis, D. & Ward, Andrew B. Model Building and Refinement of a Natively Glycosylated HIV-1 Env Protein by High-Resolution Cryoelectron Microscopy. Structure 23, 1943-1951, doi:https://doi.org/10.1016/j.str.2015.07.020 (2015).
59 Scharf, L. et al. Broadly Neutralizing Antibody 8ANC195 Recognizes Closed and Open States of HIV-1 Env. Cell 162, 1379-1390, doi:https://doi.org/10.1016/j.cell.2015.08.035 (2015).
60 Jardine, J. G. et al. Minimally Mutated HIV-1 Broadly Neutralizing Antibodies to Guide Reductionist Vaccine Design. PLOS Pathogens 12, e1005815, doi:10.1371/journal.ppat.1005815 (2016).
61 Stewart-Jones, Guillaume B. E. et al. Trimeric HIV-1-Env Structures Define Glycan Shields from Clades A, B, and G. Cell 165, 813-826, doi:10.1016/j.cell.2016.04.010.
62 Gristick, H. B. et al. Natively glycosylated HIV-1 Env structure reveals new mode for antibody recognition of the CD4-binding site. Nature Structural & Amp; Molecular Biology 23, 906, doi:10.1038/nsmb.3291
https://www.nature.com/articles/nsmb.3291#supplementary-information (2016).
63 Zhou, T. et al. Quantification of the Impact of the HIV-1-Glycan Shield on Antibody Elicitation. Cell Reports 19, 719-732, doi:https://doi.org/10.1016/j.celrep.2017.04.013 (2017).
64 Lee, J. H. et al. A Broadly Neutralizing Antibody Targets the Dynamic HIV Envelope Trimer Apex via a Long, Rigidified, and Anionic β-Hairpin Structure. Immunity 46, 690-702, doi:https://doi.org/10.1016/j.immuni.2017.03.017 (2017).
65 Barnes, C. O. et al. Structural characterization of a highly-potent V3-glycan broadly neutralizing antibody bound to natively-glycosylated HIV-1 envelope. Nature Communications 9, 1251, doi:10.1038/s41467-018-03632-y (2018).
66 Dingens, A. S. et al. Complete functional mapping of infection- and vaccine-elicited antibodies against the fusion peptide of HIV. PLOS Pathogens 14, e1007159, doi:10.1371/journal.ppat.1007159 (2018).
67 Tria, G., Mertens, H. D. T., Kachala, M. & Svergun, D. I. Advanced ensemble modelling of flexible macromolecules using X-ray solution scattering. IUCrJ 2, 207-217, doi:doi:10.1107/S205225251500202X (2015).
68 Lee, J. H., Ozorowski, G. & Ward, A. B. Cryo-EM structure of a native, fully glycosylated, cleaved HIV-1 envelope trimer. Science 351, 1043, doi:10.1126/science.aad2450 (2016).

69 Cao, L. et al. Differential processing of HIV envelope glycans on the virus and soluble recombinant trimer. Nature Communications 9, 3693, doi:10.1038/s41467-018-06121-4 (2018).
70 Humphrey, W., Dalke, A. & Schulten, K. VMD: Visual molecular dynamics. Journal of Molecular Graphics 14, 33-38, doi:https://doi.org/10.1016/0263-7855(96)00018-5 (1996).
71 Team, R. C. R: A Language and Environment for Statistical Computing. (2017).
72 Juette, M. F. et al. Single-molecule imaging of non-equilibrium molecular ensembles on the millisecond timescale. Nature methods 13, 341-344, doi:10.1038/nmeth.3769 (2016).

1-16. (canceled)

17. A nucleic acid encoding a modified recombinant HIV-1 envelope comprising an isoleucine residue at amino acid position 68, a valine residue at amino acid position 204, a leucine residue at amino acid position 208, and a leucine residue at amino acid position 255, wherein the amino acid positions refer to HXB2 numbering in an unmodified HIV-1 envelope.

18. The nucleic acid of claim 17, wherein the modified recombinant HIV-1 envelope is a gp120 envelope, a gp140 envelope, or a gp160 envelope.

19. The nucleic acid of claim of claim 17, wherein the modified recombinant HIV-1 envelope further comprises a methionine residue at amino acid position 320, a methionine residue at amino acid position 422, a methionine residue at amino acid position 203, a leucine residue at amino acid position 302, and a leucine residue at amino acid position 300, wherein the amino acid positions refer to HXB2 numbering in an unmodified HIV-1 envelope.

20. The nucleic acid of claim 19, wherein the unmodified HIV-1 envelope is an envelope selected from: CH505 M5G458mut envelope, BG505, CONs, JRFL, CH505 T/F, w.53., CH505 MS, CH505 Ml 1, CH848 10.17 DT or 19CV3.

21. The nucleic acid of claim 17, wherein the unmodified HIV-1 envelope is an envelope selected from: CH505 M5G458mut envelope, BG505, CONs, JRFL, CH505 T/F, w.53., CH505 MS, CH505 Ml 1, CH848 10.17 DT or 19CV3.

22. The nucleic acid of claim 17, wherein the modified recombinant HIV-1 envelope further comprises additional stabilizing mutations.

23. The nucleic acid of claim 17, wherein the modified recombinant HIV-1 envelope is a CH505 M5G458mut envelope comprising the amino acid sequence of SEQ ID NO: 51.

24. The nucleic acid of claim 17, wherein the modified recombinant HIV-1 envelope is a membrane bound gp160 envelope.

25. An immunogenic composition comprising the nucleic acid of claim 17.

26. The immunogenic composition of claim 25, wherein the nucleic acid comprises a DNA or an RNA.

27. The immunogenic composition of claim 26, wherein the RNA is an mRNA.

28. The immunogenic composition of claim 25, further comprising a carrier.

29. The immunogenic composition of 28, further comprising an adjuvant.

30. The immunogenic composition of claim 25, wherein the nucleic acid is inserted in a vector.

31. The immunogenic composition of claim 25, wherein the nucleic acid is comprised in a nanoparticle.


SEQUENCE LISTING (64 sequences)

SEQ ID NO: 1 - protein, 4 aa, unidentified
  REGION 1..4: note = Description of Unknown: cleavage site sequence
REKR

SEQ ID NO: 2 - protein, 9 aa, unidentified
  REGION 1..9: note = Description of Unknown: cleavage site sequence
RRVVEREKR

SEQ ID NO: 3 - protein, 9 aa, unidentified
  REGION 1..9: note = Description of Unknown: cleavage site sequence
ERVVEREKE

SEQ ID NO: 4 - protein, 4 aa, unidentified
  REGION 1..4: note = Description of Unknown: cleavage site sequence
SEKS

SEQ ID NO: 5 - protein, 79 aa, unidentified
  REGION 1..79: note = Description of Unknown: envelope sequence
MRVMGIQRNYPQWWIWSMLGFWMLMICNGMWVTVYYGVPVWKEAKTTLFCASDAKAYEKEVHNVWATHACVPTDPNPQE

SEQ ID NO: 6 - protein, 24 aa, Homo sapiens
MPMGSLQPLATLYLLGMLVASVLA

SEQ ID NO: 7 - protein, 5 aa, unidentified
  REGION 1..5: note = Description of Unknown: recognition motif sequence
  VAR_SEQ 3: note = Any amino acid
LPXTG

SEQ ID NO: 8 - protein, 5 aa, synthetic construct
  REGION 1..5: note = Description of Artificial Sequence: Synthetic peptide
GGGGG

SEQ ID NO: 9 - protein, 6 aa, synthetic construct
  REGION 1..6: note = Description of Artificial Sequence: Synthetic peptide
GQQQLG

SEQ ID NO: 10 - protein, 12 aa, synthetic construct
  REGION 1..12: note = Description of Artificial Sequence: Synthetic peptide
GDSLDMLEWSLM

SEQ ID NO: 11 - protein, 75 aa, Human immunodeficiency virus 1
ETTLFCASDAKAYETEKHNVWATHACVPTDPNPQEIHLENVTEEFNMWKNNMVEQMHTDIISLWDQSLKPCVKLT

SEQ ID NO: 12 - protein, 126 aa, Human immunodeficiency virus 1
TSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIG

SEQ ID NO: 13 - protein, 47 aa, Human immunodeficiency virus 1
GEFFYCNTSGLFNSTWISNTSVQGSNSTGSNDSITLPCRIKQIINMW

SEQ ID NO: 14 - protein, 75 aa, synthetic construct
  REGION 1..75: note = Description of Artificial Sequence: Synthetic polypeptide
ETTLFCASDAKAYETEKHNIWATHACVPTDPNPQEIHLENVTEEFNMWKNNMVEQMHTDIISLWDQSLKPCVKLT

SEQ ID NO: 15 - protein, 126 aa, synthetic construct
  REGION 1..126: note = Description of Artificial Sequence: Synthetic polypeptide
TSAITQVCPKLSFEPIPIHYCAPAGFAILKCKDKKFNGTGPCPSVSTVQCTHGIKPVLSTQLLLNGSLAEEEVMIRSENITNNAKNILVQFNTPVQINCTRPNNNTRKSIRIGPGQAFYATGDIIG

SEQ ID NO: 16 - protein, 47 aa, synthetic construct; identical to SEQ ID NO: 13
  REGION 1..47: note = Description of Artificial Sequence: Synthetic polypeptide

SEQ ID NO: 17 - protein, 75 aa, synthetic construct; identical to SEQ ID NO: 14
  REGION 1..75: note = Description of Artificial Sequence: Synthetic polypeptide

SEQ ID NO: 18 - protein, 126 aa, synthetic construct
  REGION 1..126: note = Description of Artificial Sequence: Synthetic polypeptide
TSAITMVCPKLSFEPIPIHYCAPAGFAILKCKDKKFNGTGPCPSVSTVQCTHGIKPVLSTQLLLNGSLAEEEVMIRSENITNNAKNILVQFNTPVQINCTRPLNLTRKSIRIGPGQAFYAMGDIIG

SEQ ID NO: 19 - protein, 47 aa, synthetic construct
  REGION 1..47: note = Description of Artificial Sequence: Synthetic polypeptide
GEFFYCNTSGLFNSTWISNTSVQGSNSTGSNDSITLPCRIKMIINMW

SEQ ID NO: 20 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 21 - DNA, 1968 bp, synthetic construct
  misc_feature 1..1968: note = Description of Artificial Sequence: Synthetic polynucleotide
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacaagcgccgtgaccatggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaaccggaatctgtccgagatctgggataacatgacctggctgcagtgggacaaagaaatcagcaactacacccagatcatctacggcctgctggaagagagccagaaccagcaagagaaaaacgagcaggacctgctggccctggactgataaggatcc

SEQ ID NO: 22 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGAKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 23 - DNA, 1968 bp, synthetic construct
  misc_feature 1..1968: note = Description of Artificial Sequence: Synthetic polynucleotide
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacaagcgccgtgaccatggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtcgtggaaatccagccactgggaatcgccccaaccggcgctaagagaagagtggtggaacggcgaagaaggcggagagctgctggactgggtgctctgttcctgggctttcttggagccgccggatctacaatgggagccgcctctatcaccctgaccgtgcaggctagacagctgctgagcggaattgtgcagcagcagagcaacctgctgagagcccctgaagcacagcagcacatgctgcagctgacagtgtggggcatcaaacagctgcaggccagagtgctggccctggaaagatacctgaaggatcagcagctcctcggcatgtggggctgttctggcaagctgatctgctgcaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggactgatgaggatcc

SEQ ID NO: 24 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 25 - DNA, 1968 bp, synthetic construct
  misc_feature 1..1968: note = Description of Artificial Sequence: Synthetic polynucleotide
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacctccgccgtgacacaagtgtgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagcagctggtccaaccggaatctgtccgagatctgggataacatgacctggctgcagtgggacaaagaaatcagcaactacacccagatcatctacggcctgctggaagagagccagaaccagcaagagaaaaacgagcaggacctgctggccctggactgataaggatcc

SEQ ID NO: 26 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGAKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 27 - DNA, 1968 bp, synthetic construct
  misc_feature 1..1968: note = Description of Artificial Sequence: Synthetic polynucleotide
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacaagcgccgtgacaatggtctgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtcgtggaaatccagccactgggaatcgccccaaccggcgctaagagaagagtggtggaacggcgaagaaggcggagagctgctggactgggtgctctgttcctgggctttcttggagccgccggatctacaatgggagccgcctctatcaccctgaccgtgcaggctagacagctgctctctggaatcgtgcagcagcagagcaacctgctgagagcccctgaagctcagcagcacatgctgcagctgacagtgtggggcatcaaacagctgcaggccagagtgctggccctggaaagatacctgaaggatcagcagctcctcggcatgtggggctgttctggcaagctgatctgctgcaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggactgatgaggatcc

SEQ ID NO: 28 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 29 - DNA, 1968 bp, synthetic construct
  misc_feature 1..1968: note = Description of Artificial Sequence: Synthetic polynucleotide
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacctccgccgtgacacaagtgtgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtggaaatccagccactgggaatcgccccaaccggcgctaagagaagagtggtcgagcggagaagaaggcggagagctgctggactgggtgccctgtttctgggctttcttggagccgccggaagcacaatgggagccgcctctattaccctgaccgtgcaggctagacagctgctctctggaatcgtgcagcagcagagcaacctgctgagagcccctgaagctcagcagcacatgctgcagctgacagtgtggggaatcaagcagctgcaggccagagtgctggccctggaaagatacctgaaggatcagcagctcctcggcatgtggggctgttctggcaagctgatctgctgcaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggactgatgaggatcc

SEQ ID NO: 30 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 31 - protein, 648 aa, synthetic construct; identical to SEQ ID NO: 24
  REGION 1..648: note = Description of Artificial Sequence: Synthetic polypeptide

SEQ ID NO: 32 - protein, 648 aa, synthetic construct; identical to SEQ ID NO: 20
  REGION 1..648: note = Description of Artificial Sequence: Synthetic polypeptide

SEQ ID NO: 33 - protein, 648 aa, synthetic construct; identical to SEQ ID NO: 30
  REGION 1..648: note = Description of Artificial Sequence: Synthetic polypeptide

SEQ ID NO: 34 - protein, 648 aa, synthetic construct
  REGION 1..648: note = Description of Artificial Sequence: Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 35 - protein, 648 aa, synthetic construct; identical to SEQ ID NO: 28
  REGION 1..648: note = Description of Artificial Sequence: Synthetic polypeptide

SEQ ID NO: 36 - protein, 648 aa, synthetic construct; identical to SEQ ID NO: 26
  REGION 1..648: note = Description of Artificial Sequence: Synthetic polypeptide

SEQ ID NO: 37 - protein, 648 aa, synthetic construct; identical to SEQ ID NO: 22
  REGION 1..648: note = Description of Artificial Sequence: Synthetic polypeptide

SEQ ID NO: 38 - protein, 648 aa, synthetic construct; identical to SEQ ID NO: 28
  REGION 1..648: note = Description of Artificial Sequence: Synthetic polypeptide

SEQ ID NO: 39 - protein, 648 aa, synthetic construct; identical to SEQ ID NO: 26
  REGION 1..648: note = Description of Artificial Sequence: Synthetic polypeptide

SEQ ID NO: 40 - protein, 648 aa, synthetic construct; identical to SEQ ID NO: 20
  REGION 1..648: note = Description of Artificial Sequence: Synthetic polypeptide

SEQ ID NO: 41 - protein, 648 aa, synthetic construct; identical to SEQ ID NO: 22
  REGION 1..648: note = Description of Artificial Sequence: Synthetic polypeptide

SEQ ID NO: 42 - protein, 648 aa, synthetic construct; identical to SEQ ID NO: 24
  REGION 1..648: note = Description of Artificial Sequence: Synthetic polypeptide

SEQ ID NO: 43 - protein, 648 aa, synthetic construct; identical to SEQ ID NO: 26
  REGION 1..648: note = Description of Artificial Sequence: Synthetic polypeptide

SEQ ID NO: 44 - protein, 648 aa, synthetic construct; identical to SEQ ID NO: 28
  REGION 1..648: note = Description of Artificial Sequence: Synthetic polypeptide

SEQ ID NO: 45 - protein, 624 aa, synthetic construct
  REGION 1..624: note = Description of Artificial Sequence: Synthetic polypeptide
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 46 - protein, 624 aa, synthetic construct
  REGION 1..624: note = Description of Artificial Sequence: Synthetic polypeptide
AENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 47 - protein, 624 aa, synthetic construct
  REGION 1..624: note = Description of Artificial Sequence: Synthetic polypeptide
AENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 48 - DNA, 1965 bp, synthetic construct
  misc_feature 1..1965: note = Description of Artificial Sequence: Synthetic polynucleotide
  source 1..1965: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacaagcgccgtgacaatggtctgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaaccggaatctgtccgagatctgggataacatgacctggctgcagtgggacaaagaaatcagcaactacacccagatcatctacggcctgctggaagagagccagaaccagcaagagaaaaacgagcaggacctgctggccctggactgaggatcc

SEQ ID NO: 49 - protein, 623 aa, synthetic construct
  REGION 1..623: note = Description of Artificial Sequence: Synthetic polypeptide
AENLWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITKNVKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDYGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 50 - protein, 623 aa, synthetic construct
  REGION 1..623: note = Description of Artificial Sequence: Synthetic polypeptide
AENLWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITMACPKVSFDPIPIHYCAPAGYAILKCNNKTFTGTGPCNNVSTVQCTHGIKPVVSTQLLLNGSLAEGEIIIRSENITKNVKTIIVHLNESVKIECTRPLNLTRTSIRIGPGQAFYAMGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKMIINMWQEVGRAMYAPPIAGNITCISNITGLLLTRDYGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 51 - protein, 623 aa, synthetic construct
  REGION 1..623: note = Description of Artificial Sequence: Synthetic polypeptide
AENLWVTVYYGVPVWKEAKTTLFCASDAKAYEKEVHNIWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQVCPKLSFDPIPIHYCAPAGYAILKCNNKTFTGTGPCNNVSTVQCTHGIKPVLSTQLLLNGSLAEGEIIIRSENITKNVKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDYGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 52 - protein, 623 aa, synthetic construct
  REGION 1..623: note = Description of Artificial Sequence: Synthetic polypeptide
AENLWVTVYYGVPVWKEAKTTLFCASDAKAYEKEVHNIWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITMVCPKLSFDPIPIHYCAPAGYAILKCNNKTFTGTGPCNNVSTVQCTHGIKPVLSTQLLLNGSLAEGEIIIRSENITKNVKTIIVHLNESVKIECTRPLNLTRTSIRIGPGQAFYAMGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKMIINMWQEVGRAMYAPPIAGNITCISNITGLLLTRDYGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 53 - protein, 860 aa, synthetic construct
  REGION 1..860: note = Description of Artificial Sequence: Synthetic polypeptide
MRVMGIQRNCQHLFRWGTMILGMIIICSAAENLWVTVYYGVPVWKDAETTLFCASDAKAYETEKHNIWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITMVCPKLSFEPIPIHYCAPAGFAILKCKDKKFNGTGPCPSVSTVQCTHGIKPVLSTQLLLNGSLAEEEVMIRSENITNNAKNILVQFNTPVQINCTRPLNLTRKSIRIGPGQAFYAMGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKMIINMWQRIGQAMYAPPIQGVIRCVSNITGLILTRDGGSTNSTTETFRPGGGDMRDNWRSELYKYKVVKIEPLGVAPTRAKRRVVGREKRAVGIGAVFLGFLGAAGSTMGAASMTLTVQARNLLSGIVQQQSNLLRAIEAQQHLLKLTVWGIKQLQARVLAVERYLRDQQLLGIWGCSGKLICTTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLSFQTHTPNPRGLDRPERIEEEDGEQDRGRSTRLVSGFLALAWDDLRSLCLFCYHRLRDFILIAARIVELLGHSSLKGLRLGWEGLKYLWNLLAYWGRELKISAINLFDTIAIAVAEWTDRVIEIGQRLCRAFLHIPRRIRQGLERALL

SEQ ID NO: 54 - protein, 846 aa, synthetic construct
  REGION 1..846: note = Description of Artificial Sequence: Synthetic polypeptide
MRVMGIQRNYPQWWIWSMLGFWMLMICNGMWVTVYYGVPVWKEAKTTLFCASDAKAYEKEVHNIWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITMVCPKLSFDPIPIHYCAPAGYAILKCNNKTFTGTGPCNNVSTVQCTHGIKPVLSTQLLLNGSLAEGEIIIRSENITKNVKTIIVHLNESVKIECTRPLNLTRTSIRIGPGQAFYAMGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKMIINMWQEVGRAMYAPPIAGNITCISNITGLLLTRDYGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 55 - protein, 847 aa, synthetic construct
  REGION 1..847: note = Description of Artificial Sequence: Synthetic polypeptide
MGILKNYPQWWIWGILGFWMLMICNGKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSAVTMVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 56 - protein, 658 aa, synthetic construct
  REGION 1..658: note = Description of Artificial Sequence: Synthetic polypeptide
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 57 - protein, 851 aa, synthetic construct
  REGION 1..851: note = Description of Artificial Sequence: Synthetic polypeptide
MRVTGILRNYPQWWIWGILGFWMLMTCNGEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 58 - protein, 647 aa, synthetic construct
  REGION 1..647: note = Description of Artificial Sequence: Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 59 - DNA, 2601 bp, synthetic construct
  misc_feature 1..2601: note = Description of Artificial Sequence: Synthetic polynucleotide
  source 1..2601: mol_type = other DNA
aagcttgtcgacgccaccatgcctatgggaagcctgcaacctctggccacactgtacctgctgggaatgctggtggcttctgtgctggccgccgagaatctgtgggtcacagtgtactatggcgtgcccgtgtggaaggacgccgagacaacactgttttgtgccagcgacgccaaggcctacgagacagagaagcacaacatctgggccactcacgcctgcgtgccaaccgatcctaatcctcaagagatccacctggaaaacgtgaccgaggaattcaacatgtggaagaacaacatggtcgagcagatgcacaccgacatcatcagcctgtgggaccagagcctgaagccttgcgtgaagctgacccctctgtgtgtgaccctgcagtgcaccaacgtgaccaacaacatcaccgacgacatgcggggcgagctgaagaactgcagcttcaacatgaccaccgagctgcgggacaagaaacagaaggtgtacagcctgttctaccggctggacgtggtgcagatcaacgagaaccagggcaacagaagcaacaacagcaacaaagagtaccggctgatcaactgcaacaccagcgccatcactcaggtgtgtcctaagctgtccttcgagcccattcctatccactactgtgcccctgccggcttcgccatcctgaagtgcaaggacaagaagttcaacggcacaggcccctgtccttccgtgtctaccgtgcagtgtacccacggcatcaagcccgtgctgtctacacagctgctgctgaatggcagcctggccgaagaggaagtgatgatcagaagcgagaacatcaccaacaacgccaagaacatcctggtccagttcaacacccctgtgcagattaactgtacccggcctaacaacaacacccggaagtccatcagaatcggcccaggccaggccttttatgccaccggcgatatcatcggcgacatcagacaggcccactgcaacgtgtccaaggccacatggaatgagacactgggcaaagtggtcaagcagctgcggaagcacttcggcaacaataccatcatcagattcgccaacagctctggcggcgacctggaagtgaccacacacagctttaactgtggcggcgagttcttctactgcaatacctccggcctgttcaacagcacctggatcagcaacacaagcgtgcagggcagcaatagcaccggcagcaacgacagcatcaccctgccttgccggatcaagcagatcatcaatatgtggcagcggatcggacaggctatgtacgcccctcctattcagggcgtgatcagatgcgtgtccaatatcaccggcctgatcctgaccagagatggcggcagcaccaactccaccaccgagacttttagacccggcggaggcgacatgagagacaattggagaagcgagctgtacaagtacaaggtggtcaagatcgagcccctgggcgtcgcacctaccagagccaagagaagagtcgtgggccgcgagaagagagccgttggaattggagccgtgttcctgggctttctgggagccgctggatctacaatgggcgctgccagcatgacactgaccgtgcaggctagaaatctgctgagcggcatcgtgcagcagcagagcaatctgctgcgggccattgaagcccagcagcatctgctgaaactgacagtgtggggcatcaaacagctgcaggccagagtgctggctgtggaaagatacctgagggaccagcagctcctcggcatctggggatgttctggcaagctgatctgtaccaccaatgtgccctggaacagcagctggtccaaccggaatctgagcgagatctgggacaatatgacctggctgcagtgggacaaagagatctccaactacacccagatcatctacggcctgctggaagagtcccagaaccagcaagagaaaaacgagcaggacctgctggccctggataagtgggctagcctgtggaattggttcgacatcagcaattggctgtggtacatcaagatcttcatcatgatcgtcggcggactgatcggcctgagaatcgtgtttgccgtgctgagcgtgatccacagagtgcggcagggatatagccctctgagcttccagacacacacccctaatcctagaggcctggacagacccgagcggatcgaagaagaggacggcgaacaggacagaggcagaagcaccagactggtttccggcttcctggctctggcttgggacgatctgagaagcctgtgcctgttctgctaccaccggctgagagactttatcctgattgccgccaggatcgtggaactgctgggacacagctctctgaagggcctgagactcggatgggagggcctgaagtatctgtggaacctgctcgcctactggggaagagagctgaaaatctccgccatcaacctgttcgacacaatcgccattgccgtggccgagtggaccgacagagtgatcgagattggccagagactgtgccgggccttcctgcacatccctcggagaattagacagggcctcgaacgggccctgctgtgataaggatcctctaga

SEQ ID NO: 60 - DNA, 2601 bp, synthetic construct
  misc_feature 1..2601: note = Description of Artificial Sequence: Synthetic polynucleotide
  source 1..2601: mol_type = other DNA
aagcttgtcgacgccaccatgcctatgggaagcctgcaacctctggccacactgtacctgctgggaatgctggtggcttctgtgctggccgccgagaatctgtgggtcacagtgtactatggcgtgcccgtgtggaaggacgccgagacaacactgttttgtgccagcgacgccaaggcctacgagacagagaagcacaacgtgtgggccactcacgcctgcgtgccaaccgatcctaatcctcaagagatccacctggaaaacgtgaccgaggaattcaacatgtggaagaacaacatggtcgagcagatgcacaccgacatcatcagcctgtgggaccagagcctgaagccttgcgtgaagctgacccctctgtgtgtgaccctgcagtgcaccaacgtgaccaacaacatcaccgacgacatgcggggcgagctgaagaactgcagcttcaacatgaccaccgagctgcgggacaagaaacagaaggtgtacagcctgttctaccggctggacgtggtgcagatcaacgagaaccagggcaacagaagcaacaacagcaacaaagagtaccggctgatcaactgcaacaccagcgccatcactatggcctgtcctaaggtgtccttcgagcccattcctatccactactgtgcccctgccggcttcgccatcctgaagtgcaaggacaagaagttcaacggcacaggcccctgtccttccgtgtctaccgtgcagtgtacccacggcatcaagcccgtggtgtctacacagctgctgctgaatggcagcctggccgaagaggaagtgatgatcagaagcgagaacatcaccaacaacgccaagaacatcctggtccagttcaacacccctgtgcagattaactgtacccggcctctgaacctgacccggaagtccatcagaatcggcccaggccaggccttttatgccatgggcgatatcatcggcgacatcagacaggcccactgcaacgtgtccaaggccacatggaatgagacactgggcaaagtggtcaagcagctgcggaagcacttcggcaacaataccatcatcagattcgccaacagctctggcggcgacctggaagtgaccacacacagctttaactgtggcggcgagttcttctactgcaatacctccggcctgttcaacagcacctggatcagcaacacaagcgtgcagggcagcaatagcaccggcagcaacgacagcatcaccctgccttgccggatcaagatgatcatcaatatgtggcagcggatcggacaggctatgtacgcccctcctattcagggcgtgatcagatgcgtgtccaatatcaccggcctgatcctgaccagagatggcggcagcaccaactccaccaccgagacttttagacccggcggaggcgacatgagagacaattggagaagcgagctgtacaagtacaaggtggtcaagatcgagcccctgggcgtcgcacctaccagagccaagagaagagtcgtgggccgcgagaagagagccgttggaattggagccgtgttcctgggctttctgggagccgctggatctacaatgggcgctgccagcatgacactgaccgtgcaggctagaaatctgctgagcggcatcgtgcagcagcagagcaatctgctgcgggccattgaagcccagcagcatctgctgaaactgacagtgtggggcatcaaacagctgcaggccagagtgctggctgtggaaagatacctgagggaccagcagctcctcggcatctggggatgttctggcaagctgatctgtaccaccaatgtgccctggaacagcagctggtccaaccggaatctgagcgagatctgggacaatatgacctggctgcagtgggacaaagagatctccaactacacccagatcatctacggcctgctggaagagtcccagaaccagcaagagaaaaacgagcaggacctgctggccctggataagtgggctagcctgtggaattggttcgacatcagcaattggctgtggtacatcaagatcttcatcatgatcgtcggcggactgatcggcctgagaatcgtgtttgccgtgctgagcgtgatccacagagtgcggcagggatatagccctctgagcttccagacacacacccctaatcctagaggcctggacagacccgagcggatcgaagaagaggacggcgaacaggacagaggcagaagcaccagactggtttccggcttcctggctctggcttgggacgatctgagaagcctgtgcctgttctgctaccaccggctgagagactttatcctgattgccgccaggatcgtggaactgctgggacacagctctctgaagggcctgagactcggatgggagggcctgaagtatctgtggaacctgctcgcctactggggaagagagctgaaaatctccgccatcaacctgttcgacacaatcgccattgccgtggccgagtggaccgacagagtgatcgagattggccagagactgtgccgggccttcctgcacatccctcggagaattagacagggcctcgaacgggccctgctgtgataaggatcctctaga

SEQ ID NO: 61 - DNA, 2601 bp, synthetic construct
  misc_feature 1..2601: note = Description of Artificial Sequence: Synthetic polynucleotide
  source 1..2601: mol_type = other DNA
aagcttgtcgacgccaccatgcctatgggaagcctgcaacctctggccacactgtacctgctgggaatgctggtggcttctgtgctggccgccgagaatctgtgggtcacagtgtactatggcgtgcccgtgtggaaggacgccgagacaacactgttttgtgccagcgacgccaaggcctacgagacagagaagcacaacatctgggccactcacgcctgcgtgccaaccgatcctaatcctcaagagatccacctggaaaacgtgaccgaggaattcaacatgtggaagaacaacatggtcgagcagatgcacaccgacatcatcagcctgtgggaccagagcctgaagccttgcgtgaagctgacccctctgtgtgtgaccctgcagtgcaccaacgtgaccaacaacatcaccgacgacatgcggggcgagctgaagaactgcagcttcaacatgaccaccgagctgcgggacaagaaacagaaggtgtacagcctgttctaccggctggacgtggtgcagatcaacgagaaccagggcaacagaagcaacaacagcaacaaagagtaccggctgatcaactgcaacaccagcgccatcactatggtgtgtcctaagctgtccttcgagcccattcctatccactactgtgcccctgccggcttcgccatcctgaagtgcaaggacaagaagttcaacggcacaggcccctgtccttccgtgtctaccgtgcagtgtacccacggcatcaagcccgtgctgtctacacagctgctgctgaatggcagcctggccgaagaggaagtgatgatcagaagcgagaacatcaccaacaacgccaagaacatcctggtccagttcaacacccctgtgcagattaactgtacccggcctctgaacctgacccggaagtccatcagaatcggcccaggccaggccttttatgccatgggcgatatcatcggcgacatcagacaggcccactgcaacgtgtccaaggccacatggaatgagacactgggcaaagtggtcaagcagctgcggaagcacttcggcaacaataccatcatcagattcgccaacagctctggcggcgacctggaagtgaccacacacagctttaactgtggcggcgagttcttctactgcaatacctccggcctgttcaacagcacctggatcagcaacacaagcgtgcagggcagcaatagcaccggcagcaacgacagcatcaccctgccttgccggatcaagatgatcatcaatatgtggcagcggatcggacaggctatgtacgcccctcctattcagggcgtgatcagatgcgtgtccaatatcaccggcctgatcctgaccagagatggcggcagcaccaactccaccaccgagacttttagacccggcggaggcgacatgagagacaattggagaagcgagctgtacaagtacaaggtggtcaagatcgagcccctgggcgtcgcacctaccagagccaagagaagagtcgtgggccgcgagaagagagccgttggaattggagccgtgttcctgggctttctgggagccgctggatctacaatgggcgctgccagcatgacactgaccgtgcaggctagaaatctgctgagcggcatcgtgcagcagcagagcaatctgctgcgggccattgaagcccagcagcatctgctgaaactgacagtgtggggcatcaaacagctgcaggccagagtgctggctgtggaaagatacctgagggaccagcagctcctcggcatctggggatgttctggcaagctgatctgtaccaccaatgtgccctggaacagcagctggtccaaccggaatctgagcgagatctgggacaatatgacctggctgcagtgggacaaagagatctccaactacacccagatcatctacggcctgctggaagagtcccagaaccagcaagagaaaaacgagcaggacctgctggccctggataagtgggctagcctgtggaattggttcgacatcagcaattggctgtggtacatcaagatcttcatcatgatcgtcggcggactgatcggcctgagaatcgtgtttgccgtgctgagcgtgatccacagagtgcggcagggatatagccctctgagcttccagacacacacccctaatcctagaggcctggacagacccgagcggatcgaagaagaggacggcgaacaggacagaggcagaagcaccagactggtttccggcttcctggctctggcttgggacgatctgagaagcctgtgcctgttctgctaccaccggctgagagactttatcctgattgccgccaggatcgtggaactgctgggacacagctctctgaagggcctgagactcggatgggagggcctgaagtatctgtggaacctgctcgcctactggggaagagagctgaaaatctccgccatcaacctgttcgacacaatcgccattgccgtggccgagtggaccgacagagtgatcgagattggccagagactgtgccgggccttcctgcacatccctcggagaattagacagggcctcgaacgggccctgctgtgataaggatcctctaga

SEQ ID NO: 62 - DNA, 2565 bp, synthetic construct
  misc_feature 1..2565: note = Description of Artificial Sequence: Synthetic polynucleotide
  source 1..2565: mol_type = other DNA
aagcttgtcgacgccaccatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacatctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacctccgccgtgacacaggtgtgccccaaactgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtgctgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtggaaatccagccactgggaatcgccccaaccggcgctaagagaagagtggtggaacgcgagaaaagagccgctggactgggagccctgttcctgggatttcttggagccgccggaagcacaatgggagccgcctctattaccctgaccgtgcaggctagacagctgctgagcggaattgtgcagcagcagagcaacctgctgagagccattgaagcccagcagcacatgctgcagctgaccgtgtggggaatcaaacagctgcaggccagagtgctggccctggaaagatacctgaaggaccagcagctcctcggcatgtggggctgttctggcaagctgatctgcaccaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggacagctggaatagcctgtggaactggttcagcatcaccaagtggctgtggtacatcaagatcttcatcatgatcgtcggcggcctgatcggcctgagaatcgtgtttgccgtgctgagcatcgtgaacagagtgcggcagggatacagcccactgagcctgcaaaccctgacacctaatcctagagagcccgaccggctgagaggcatcgaagaagaaggcggcgagcaggatcgggacagatccatcagactggtgtccggcttcctgcctatcgtgtgggacgatctgagaagcctgtgcctgttcagctaccaccggctgcgggattttctgctgcttgccgccagagtggttgaactgctgggcagatctagcctgaggggcctgcaaagaggctgggaagtcctgaagtacctgggcagcctggtgcagtattggggcctcgagctgaaaaagagcgccattagcctgttcgataccctggccattgctgtggccgagggcaccgatagaatcattgagctgatccagggcttctgccgggccatcagaaacatccccaccagaatcagacagggcttcgaggccagcctgctgtgataaggatcctctaga

SEQ ID NO: 63 - DNA, 2565 bp, synthetic construct
  misc_feature 1..2565: note = Description of Artificial Sequence: Synthetic polynucleotide
  source 1..2565: mol_type = other DNA
aagcttgtcgacgccaccatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacctccgccgtgacaatggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctctgaacctgacccggaagtctgtgcggatcggccctggccagacattctatgccatgggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcatcaatatgtggcaaggcgtgggcagagctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtggaaatccagccactgggaatcgccccaaccggcgctaagagaagagtggtggaacgcgagaaaagagccgctggactgggagccctgttcctgggatttcttggagccgccggaagcacaatgggagccgcctctattaccctgaccgtgcaggctagacagctgctgagcggaattgtgcagcagcagagcaacctgctgagagccattgaagcccagcagcacatgctgcagctgaccgtgtggggaatcaaacagctgcaggccagagtgctggccctggaaagatacctgaaggaccagcagctcctcggcatgtggggctgttctggcaagctgatctgcaccaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggacagctggaatagcctgtggaactggttcagcatcaccaagtggctgtggtacatcaagatcttcatcatgatcgtcggcggcctgatcggcctgagaatcgtgtttgccgtgctgagcatcgtgaacagagtgcggcagggatacagcccactgagcctgcaaaccctgacacctaatcctagagagcccgaccggctgagaggcatcgaagaagaaggcggcgagcaggatcgggacagatccatcagactggtgtccggcttcctgcctatcgtgtgggacgatctgagaagcctgtgcctgttcagctaccaccggctgcgggattttctgctgcttgccgccagagtggttgaactgctgggcagatctagcctgaggggcctgcaaagaggctgggaagtcctgaagtacctgggcagcctggtgcagtattggggcctcgagctgaaaaagagcgccattagcctgttcgataccctggccattgctgtggccgagggcaccgatagaatcattgagctgatccagggcttctgccgggccatcagaaacatccccaccagaatcagacagggcttcgaggccagcctgctgtgataaggatcctctaga

SEQ ID NO: 64 - DNA, 2565 bp, synthetic construct
  misc_feature 1..2565: note = Description of Artificial Sequence: Synthetic polynucleotide
  source 1..2565: mol_type = other DNA
aagcttgtcgacgccaccatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacatctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaaagagaagaaagagtacgccctgttctacaagcccgacatcgtgcccctgagcgagacaaacaacaccagcgagtaccggctgatcaactgcaacacctccgccgtgacaatggtgtgccccaaactgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtgctgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctctgaacctgacccggaagtctgtgcggatcggccctggccagacattctatgccatgggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcatcaatatgtggcaaggcgtgggcagagctatgtacgcccctcctatcgccggcaacatcacctgtcggagcaatatcacaggcctgctgctcaccagagatggcggcaccaatagcaacgagacagaaaccttcagacctgccggcggagacatgagagacaattggagaagcgagctgtacaagtacaaggtggtggaaatccagccactgggaatcgccccaaccggcgctaagagaagagtggtggaacgcgagaaaagagccgctggactgggagccctgttcctgggatttcttggagccgccggaagcacaatgggagccgcctctattaccctgaccgtgcaggctagacagctgctgagcggaattgtgcagcagcagagcaacctgctgagagccattgaagcccagcagcacatgctgcagctgaccgtgtggggaatcaaacagctgcaggccagagtgctggccctggaaagatacctgaaggaccagcagctcctcggcatgtggggctgttctggcaagctgatctgcaccaccaacgtgccctggaacacctcctggtccaacaagagcgaaaccgacatctgggacaacatgacctggatgcagtgggagagagagatcagcaactacaccgagacaatctacaagctgctcgaggacagccagaaccagcaagagagaaacgagcaggacctgctggctctggacagctggaatagcctgtggaactggttcagcatcaccaagtggctgtggtacatcaagatcttcatcatgatcgtcggcggcctgatcggcctgagaatcgtgtttgccgtgctgagcatcgtgaacagagtgcggcagggatacagcccactgagcctgcaaaccctgacacctaatcctagagagcccgaccggctgagaggcatcgaagaagaaggcggcgagcaggatcgggacagatccatcagactggtgtccggcttcctgcctatcgtgtgggacgatctgagaagcctgtgcctgttcagctaccaccggctgcgggattttctgctgcttgccgccagagtggttgaactgctgggcagatctagcctgaggggcctgcaaagaggctgggaagtcctgaagtacctgggcagcctggtgcagtattggggcctcgagctgaaaaagagcgccattagcctgttcgataccctggccattgctgtggccgagggcaccgatagaatcattgagctgatccagggcttctgccgggccatcagaaacatccccaccagaatcagacagggcttcgaggccagcctgctgtgataaggatcctctaga